(19) United States
(12) Patent Application Publication
(10) Pub. No.: US 2020/0181271 A1
(43) Pub. Date: Jun. 11, 2020

(54) METHODS FOR PREVENTING AND TREATING URINARY INCONTINENCE

(71) Applicant: NOVARTIS AG, Basel (CH)

(72) Inventors: Shinji HATAKEYAMA, Basel (CH); Michaela KNEISSEL, Basel (CH); Estelle TRIFILIEFF, Dietwiller (FR)

(21) Appl. No.: 16/626,170

(22) PCT Filed: Jun. 26, 2018
(86) PCT No.: PCT/IB2018/054702

§ 371 Date: Dec. 23, 2019

(30) Foreign Application Priority Data
Jun. 28, 2017 (EP) 17178429.1

(51) Int. Cl. C07K 16/28; A61P 13/00

(52) U.S. Cl. CPC C07K 16/2863; A61P 13/00; C07K 2317/21; A61K 38/00; C07K 2317/76; C07K 2317/565; C07K 2317/34

(57) ABSTRACT

The disclosure relates to novel uses and methods for preventing and/or treating urinary incontinence, which employ a therapeutically effective amount of an ActRII receptor antagonist, e.g., an ActRII receptor binding molecule, e.g., an ActRII receptor antibody, such as the bimagrumab antibody.

### TECHNICAL FIELD

This disclosure is in the field of activin receptor type II (ActRII) antagonists, e.g., molecules capable of antagonizing the binding of activins, growth differentiation factors (GDFs), bone morphogenic proteins (BMPs) and myostatin to the human ActRII receptor, e.g., an antagonist antibody to ActRIIA and/or ActRIIB, e.g., bimagrumab. In particular, it relates to treating and preventing urinary incontinence, by administering to a subject a therapeutically effective amount of an ActRII receptor antagonist.

### BACKGROUND OF THE DISCLOSURE

The activin type IIB receptor (ActRIIB) is a signaling receptor for various members of the transforming growth factor beta (TGF-β) superfamily. Members of this family include activin A, nodal, BMP2, BMP6, BMP7, BMP9, GDF5, GDF8 (myostatin) and GDF11, all of which are involved in the negative regulation of muscle (Akpan et al., 2009).

Myostatin (GDF8) acts via the activin receptor type II (mainly via ActRIIB) and its proposed signaling is through the SMAD 2/3 pathway, which is involved in the inhibition of protein synthesis, and myocyte differentiation and proliferation. Myostatin inhibition or genetic ablation increases muscle mass and strength (Lee et al 2005, Lee and McPherron 2001, Whittemore et al 2003).

Bimagrumab is the INN (international non-proprietary name) of a monoclonal antibody also known as BYM338 or MOR08159 developed to bind competitively to activin receptor type IIB (ActRIIB) with greater affinity than myostatin or activin, its natural ligands. Bimagrumab is disclosed in WO2010/125003, which is incorporated by reference herein as if fully set forth. The Bimagrumab sequences disclosed in WO2010/1253003 are listed in table 1.

Bimagrumab is a fully human antibody (modified IgG1, 234-235-Ala-Ala, λ2; The numbering of residues in the Fc region is that of the EU index of Kabat, E. A. et al., Sequences of proteins of immunological interest. 5th Edition—US Department of Health and Human Services, NIH publication no 91-3242, pp 662,680,689 (1991)) or 232-233-Ala-Ala according to the Kabat numbering system; which binds to the ligand binding domain of ActRIIA and B (bimagrumab is an ActRII binding molecule), thereby preventing binding and subsequent signaling of its ligands, including myostatin and activin that act as natural inhibitors of skeletal muscle growth.

Bimagrumab is cross-reactive with human and mouse ActRIIB and effective on human, cynomolgus, mouse and rat skeletal muscle cells. ActRIIB is widely distributed in skeletal muscle, adipose tissue and various organs, including the heart (Rebbapragada et al., Myostatin signals through a transforming growth factor 1-like signaling pathway to block adipogenesis. Molec and Cell Biol. 2003; 23:7230-7242).

Pelvic floor dysfunctions affect the pelvic region of patients. The pelvic region includes various anatomical structures, including the bladder and the urethra held in place by muscles and ligaments. When these tissues are damaged, stretched, or otherwise weakened, urinary incontinence may be the consequence. Urinary incontinence is a clinical syndrome which is defined as loss of bladder control. Urinary incontinence often results from the decrease in ability to regulate the urethra, because the interior pressure of the bladder is larger than the resistance of the urethra.

A decline in urinary continence, e.g. as a consequence of a weak sphincter, of childbirth or of prostatectomy, often causes the inability of effectively controlling the bladder. The severity of loss of bladder control ranges from increased numbers of micturitions per 24 hours, to occasionally leaking urine, to having an urge to urinate suddenly to nocturia episodes. Furthermore loss of bladder control symptoms are (i) incontinence following a sudden cough, sneezing, laughing, heavy lifting and exercise or (ii) involuntary contraction of the muscular wall of the bladder that causes an urge to urinate that cannot be stopped or (iii) bladder cannot hold as much urine as the body is making and/or the bladder cannot empty completely, causing small amounts of urinary leakage (patients experiencing constant “dribbling” of urine from the urethra).

Several types of urinary incontinence (UI) are known. For example, stress urinary incontinence (SUI) may occur as a result of sudden body movements putting pressure on the bladder. Urge urinary incontinence, e.g. people cannot hold their urine long enough to get to the toilet in time, is the results of a weakened bladder muscle. The bladder may leak urine as a consequence of indispositions or illnesses like cancer, inflammation, infections or bladder stones. Other forms of incontinence are known as reflex incontinence, psychogenic incontinence and neurogenic incontinence.

There are limited pharmacologic therapies available for the treatment of incontinence. Treatments that can be used to treat stress urinary incontinence in women are described in Rovner E S, Wein A J. Treatment options for stress urinary incontinence. Reviews in Urology 2004, 6: S29-S47. The standard of care is pelvic floor physical therapy and surgical procedures (e.g. sling; bladder neck suspension). Biological and other materials injected into the urethra have been tested for treating stress urinary incontinence symptoms with only minor success (Lee P E, Kumg R C, Drutz H P. Periurethral autologous fat injection as a treatment for female stress urinary incontinence—a randomized double-blind controlled trial. J Urol 2001, 165: 153-158). Injection of autologous muscle derived stem cells (AMDC) into the urethral sphincter in a dose escalation study showed some positive results, but only patients who received the highest dose of AMDC had statistically significant reduction in mean pad weight (Peters K M, Dmochowski R R, Carr L K, Magali R, Kaufman M R, Sirls L T, Herschorn S, Birch C, Kultgen P L, Chancellor M B. Autologous muscle derived cells for treatment of stress urinary incontinence in women. J Urol 2014, 192: 469-476.). The use of duloxetine to treat stress urinary incontinence has been tested with varying results (Norton P A, Zinner N R, Yalcin I, Bump R C. Duloxetine urinary incontinence study group. Duloxetine versus placebo in the treatment of stress urinary incontinence. Am J Obstet Gynecol 2002, 187: 40-48; Dmochowski R R, Miklos J R, Norton P A, et al. for the duloxetine urinary incontinence study group. Duloxetine versus placebo for the treatment of North America women with stress urinary incontinence. J Urol 2003, 170: 1259-1263).

The effect of testosterone on urodynamic findings and histopathomorphology of the pelvic floor muscles has been studied in rat models of stress urinary incontinence. Testosterone was found to improve leak point pressures and significantly increase the size of myofibers in treated rats, suggesting that testosterone has both preventative and curative effects on rat models of stress urinary incontinence (Mammadov R, Sinsir A, Tuglu I, Eyren V, Gurer E, Ozyurt C. The effect of testosterone treatment on urodynamic findings and histopathomorphology of pelvic floor muscles in female rats with experimentally induced stress urinary incontinence. Int Urol Nephrol 2011, 43: 1003-1008). Since free testosterone levels were also higher in the treated group, there is potential for concerns regarding side effects of supplemental steroidal testosterone in women with stress urinary incontinence.

The effects of androgens in UI have been widely studied. These studies suggest that androgens may play a substantial role in stress urinary incontinence (Bai S W, Jung Bh, Chung B C, et al. Relationship between urinary endogenous steroid metabolites and lower urinary tract function in postmenopausal women. Yonsei Med J 2003, 44: 279-287; Jung B H, Bai S W, Chung B C. Urinary profile of endogenous steroids in postmenopausal women with stress urinary incontinence. J Reprod Med 2001, 46: 969-974; Bai S W, Jung Bh, Chung B S, et al. Relationship between urinary profile of the endogenous steroids and postmenopausal women with stress urinary incontinence. Neurourol Urodynam 2003, 22: 198-204). It could be shown that increases in muscle mass resulting from exercise may cause an increases in local androgen concentrations (Aizawa K, lemitsu M, Maeda S, Mesaki N, Ushida T, Akimoto T. Endurance exercise training enhances local sex steroidogenesis in skeletal muscle. Medicine and science in sports and exercise 2011, 43(11): 2072-2080). However, the action of androgens is complex and may depend on anabolic effects, hormonal modulation, receptor expression, nitric oxide modulation, or combination of these factors (Ho M H, Bhatia N N, Bhasin S. Anabolic effects of androgens on muscles of female pelvic floor and lower urinary tract. Current Opinion in Ostetrics and Gynecology 2004, 16(5): 405-409). Anabolic steroids may increase muscle mass and strength, but have limited use because of known potential risks.

A preliminary in vivo study using an ovariectomized rat model to mimic stress urinary incontinence provides support to the potential use of SARMs for the treatment of stress urinary incontinence (Kadekawa et al, AUA Annual Meeting 2015, New Orleans, La. PD27-11). It could be demonstrated that the use of a selective androgen receptor modulator (GSK2849466A) was able to increase urethral baseline pressure (UBP) and the amplitude of urethral responses during sneezing (AURS) by 64 percent and 74 percent, respectively, as compared with the vehicle control. Histologically, the SARM treated animals had a reversal of the atrophy in urethral muscle observed in the control group.

In 1984 A. Gruneberger, N. Tommen and D. Foster reported for the first time the successful treatment of urinary incontinence in women and in children with the beta2-adrenergic clenbuterol. According to these authors, in most patients the effect of clenbuterol treatment became apparent already in the first week of treatment (Gruneberger A. Treatment of motor urge-incontinence with clenbuterol, and flavoxate hydrohloride. British Journal of Obstetrics and Gynaecology 1984; 91: 275-278). Valrlev et al., have summarized their experiences on the treatment of urinary incontinence with clenbuterol for the period 1988-1997 (B. Zozikov, S. I. Kunchev & Chr. Varlev: Application of clenbuterol in the treatment of urinary incontinence; International Urology and Nephrology 33: 413-416, 2001). Valrlev et al., pointed out that despite the fact that announcements have been made for the effects of more than 90 drugs in treating urinary incontinence, no 100% success (e.g. fully recovered ability (according to Patient Perception of Bladder Condition (PPBC) of effectively controlling the bladder, e.g. no urge to urinate suddenly or no nocturia episodes) has been reported albeit the great number of drugs. Most current treatments for urinary incontinence (UI) modulate the nervous system, and include non-selective anti-cholinergics such as oxybutynin and propantheline, or anti-muscarinics such as tolterodine, trospium, solifenacin, darifenacin, and fesoterodine. Adrenergic modulators for UI include tricyclic anti-depressants (e.g., imipramine and amitriptyline) and beta 3-adrenergic receptor agonists (e.g., mirabegron). Other urinary incontinence agents are muscle relaxants (e.g., relax the detrusor) such as flavoxate and dicyclomine. Botulinum toxins such as onabotulinumtoxin A have been used in neurogenic urinary incontinence.

Despite the number of FDA approved agents for treating urinary incontinence, there remains a need for new agents with novel mechanisms of action that normalize urethral pressure and stabilizes the urine flow and that will have a beneficial and more pronounced positive effect on stress urinary incontinence or beneficial effects on e.g. incontinence episodes per 24 hours, number of micturations per 24 hours, volume voided per incontinence, nocturia episodes per 24 hours or an improvement in patient perception of bladder condition (PPBC).

### SUMMARY OF THE DISCLOSURE

Prior to the present disclosure, targeted inhibition of activin type II receptors (ActRIIA/B) had not been contemplated or investigated as a prophylactic or therapy for urinary incontinence like stress urinary incontinence (SUI), urge urinary incontinence (UUI), reflex urinary incontinence (RUI) or (NUI) neurogenic urinary incontinence or the aforementioned conditions. As disclosed herein, there is insight that systemic administration of an ActRIIA/B receptor antagonist such as BYM338/bimagrumab, has a beneficial effect on urinary incontinence like stress urinary incontinence, urge urinary incontinence or reflex urinary incontinence. Using a dual injury childbirth simulation rat model, consisting of pudendal nerve crush (PNC) and vaginal distension (VD) which causes more severe and longer lasting damage than either PNC or VD alone in female rats (Hai-Hong Jiang et al., Dual simulated childbirth injuries result in slowed recovery of pudendal nerve and urethral function; Neurourol Urodyn. 2009; 28(3): 229-235.; Song et al., Combination Histamine and Serotonin Treatment After Simulated Childbirth Injury Improves Stress Urinary; Neurourology and Urodynamics 35:703-710 (2016)), the beneficial effect of an ActRIIA/B receptor antagonist such as Bimagrumab can be proven. As disclosed herein, it is contemplated that the ActRIIA/B receptor antagonist Bimagrumab has beneficial effect on urinary incontinence in the dual injury childbirth simulation rat model and, hence, provides the basis for the development of new ways of treating stress urinary incontinence, urge urinary incontinence or reflex urinary incontinence in humans.

Disclosed herein are ActRII receptor antagonists for use in treating urinary incontinence, in particular stress urinary incontinence, urge urinary incontinence or reflex urinary incontinence in humans. Methods using such ActRII antagonists for treating urinary incontinence, in particular stress urinary incontinence, urge urinary incontinence or reflex urinary incontinence in humans are also provided.

Disclosed herein are methods for treating and/or preventing urinary incontinence. The methods comprise administering to a subject showing symptoms of/or is suffering from urinary incontinence, or who is at risk for developing symptoms of urinary incontinence, like incontinence episodes, increased number of micturations, nocturia or a decrease in the patient bladder condition perception (PPBC) a therapeutically effective amount of an ActRII receptor antagonist, such as e.g., Bimagrumab.

The herein disclosed methods for treating and/or preventing urinary incontinence can be used to treat the following symptoms:
i. suddenly needing to empty your bladder (called urgency)
ii. having to empty your bladder more than usual (called increased urinary frequency)
iii. not being able to control when to empty your bladder (called urgency incontinence)

Disclosed herein are ActRII receptor antagonists for use in treating and/or preventing urinary incontinence. Urinary incontinence may be caused by, or associated with, pelvic floor disorders e.g. resulting from a weakened or damaged pelvic muscle.

Also disclosed herein are ActRII receptor antagonists for use in treating urinary incontinence conditions like stress urinary incontinence, urge urinary incontinence and reflex urinary incontinence.

In one embodiment the urinary incontinence treated with an ActRII receptor antagonist is related to or caused by the effects of childbirth or the menopause.

Also disclosed herein are methods for treating urinary incontinence caused by, or associated with, pelvic floor disorders e.g. resulting from a weakened or damaged pelvic muscle. The methods comprise administering to a subject showing symptoms of urinary incontinence an effective amount of an ActRII receptor antagonist.

In some instances, treatment of stress urinary incontinence (SUI), urge urinary incontinence (UUI), reflex urinary incontinence (RUI) or (NUI) neurogenic urinary incontinence as described herein result from of a weakened or damaged pelvic muscle, wherein said muscle is the musculus levator ani, musculus bulbocavernosus or musculus sphincter urethrae externus.

In one embodiment an ActRII receptor antagonist for use in treating urinary incontinence or in a method described herein is an ActRII receptor binding molecule, which can block access of ActRII-interacting ligands, such as myostatin, GDF11 and Activin A, to ActRII. The ActRII receptor binding molecule can bind to the ActRIIA and/or to the ActRIIB receptor. Examples of ActRII binding molecules include but are not limited to antibodies which bind to the ActRIIA and/or ActRIIB receptor, e.g., an anti-ActRII receptor antibody. Preferably, the anti-ActRII receptor antibody is BYM338, also known as bimagrumab.

An additional example of an ActRII receptor antagonist for use in treating urinary incontinence or in a method described herein is a soluble form of the extra-cellular domain of the ActRIIA or ActRIIB receptor, which can bind ActRII-interacting ligands, such as myostatin, GDF11 and Activin A. This “receptory-body” inhibits the function of cell-bound ActRII receptors by competing away their ligands.

Disclosed herein are ActRII receptor antagonists for use or in treating urinary incontinence or a method described herein wherein the ActRII receptor antagonist is an anti-ActRII antibody that binds to an epitope of ActRIIB consisting of amino acids 19-134 of SEQ ID NO: 181 (SEQ ID NO: 182).

Disclosed herein are ActRII receptor antagonists for use in treating urinary incontinence or in a method described herein wherein the anti-ActRII antibody binds to an epitope of ActRIIB comprising or consisting of:

(a) amino acids 78-83 of SEQ ID NO: 181
(WLDDFN-SEQ ID NO: 188);
(b) amino acids 76-84 of SEQ ID NO: 181
(GCWLDDFNC-SEQ ID NO: 186);
(c) amino acids 75-85 of SEQ ID NO: 181
(KGCWLDDFNCY-SEQ ID NO: 190);
(d) amino acids 52-56 of SEQ ID NO: 181
(EQDKR-SEQ ID NO: 189);
(e) amino acids 49-63 of SEQ ID NO: 181
(CEGEQDKRLHCYASW-SEQ ID NO: 187);
(f) amino acids 29-41 of SEQ ID NO: 181
(CIYYNANWELERT-SEQ ID NO: 191);
(g) amino acids 100-110 of SEQ ID NO: 181
(YFCCCEGNFCN-SEQ ID NO: 192);
or
(h) amino acids 78-83 of SEQ ID NO: 181
(WLDDFN)
and
amino acids 52-56 of SEQ ID NO: 181
(EQDKR).

Further anti-ActRIIB antibodies for use in treating urinary incontinence or in a method described herein include e.g.,

a) an anti-ActRIIB antibody that binds to an
epitope of ActRIIB comprising:
(a) amino acids 78-83 of SEQ ID NO: 181
(WLDDFN-SEQ ID NO: 188);
(b) amino acids 76-84 of SEQ ID NO: 181
(GCWLDDFNC-SEQ ID NO: 186);
(c) amino acids 75-85 of SEQ ID NO: 181
(KGCWLDDFNCY-SEQ ID NO: 190);
(d) amino acids 52-56 of SEQ ID NO: 181
(EQDKR-SEQ ID NO: 189);
(e) amino acids 49-63 of SEQ ID NO: 181
(CEGEQDKRLHCYASW-SEQ ID NO: 187);
(f) amino acids 29-41 of SEQ ID NO: 181
(CIYYNANWELERT-SEQ ID NO: 191);
(g) amino acids 100-110 of SEQ ID NO: 181
(YFCCCEGNFCN-SEQ ID NO: 192);
or
(h) amino acids 78-83 of SEQ ID NO: 181
(WLDDFN)
and
amino acids 52-56 of SEQ ID NO: 181
(EQDKR);
and

b) an antagonist antibody to ActRIIB that binds to an epitope of ActRIIB comprising amino acids 78-83 of SEQ ID NO: 181 (WLDDFN—SEQ ID NO:188);

(b) amino acids 76-84 of SEQ ID NO: 181
(GCWLDDFNC-SEQ ID NO: 186);
(c) amino acids 75-85 of SEQ ID NO: 181
(KGCWLDDFNCY-SEQ ID NO: 190);
(d) amino acids 52-56 of SEQ ID NO: 181
(EQDKR-SEQ ID NO: 189);
(e) amino acids 49-63 of SEQ ID NO: 181
(CEGEQDKRLHCYASW-SEQ ID NO: 187);
(f) amino acids 29-41 of SEQ ID NO: 181
(CIYYNANWELERT-SEQ ID NO: 191);
(g) amino acids 100-110 of SEQ ID NO: 181
(YFCCCEGNFCN-SEQ ID NO: 192);
or
(h) amino acids 78-83 of SEQ ID NO: 181
(WLDDFN)
and
amino acids 52-56 of SEQ ID NO: 181
(EQDKR), wherein the antibody has a KID of about
2 pM.

In one embodiment, an ActRII receptor antagonist for use in treating urinary incontinence or in a method described herein is an antibody that binds to ActRIIB with about a 10-fold or greater affinity than it binds to ActRIIA.

In another embodiment the ActRII receptor antagonist for use in treating urinary incontinence or in a method described herein may be an antibody comprising a heavy chain variable region CDR1 comprising an amino acid sequence selected from the group consisting of SEQ ID NOs: 1-14; a heavy chain variable region CDR2 comprising an amino acid sequence selected from the group consisting of SEQ ID NOs: 15-28; a heavy chain variable region CDR3 comprising an amino acid sequence selected from the group consisting of SEQ ID NOs: 29-42; a light chain variable region CDR1 comprising an amino acid sequence selected from the group consisting of SEQ ID NOs: 43-56; a light chain variable region CDR2 comprising an amino acid sequence selected from the group consisting of SEQ ID NOs: 57-70; and a light chain variable region CDR3 comprising an amino acid sequence selected from the group consisting of SEQ ID NOs: 71-84.

The ActRII receptor antagonist for use in treating urinary incontinence or in a method described herein may be an antibody comprising:

(a) a heavy chain variable region CDR1 of SEQ ID NO: 1; a heavy chain variable region CDR2 of SEQ ID NO: 15; a heavy chain variable region CDR3 of SEQ ID NO: 29; a light chain variable region CDR1 of SEQ ID NO: 43; a light chain variable region CDR2 of SEQ ID NO: 57; and a light chain variable region CDR3 of SEQ ID NO: 71,
(b) a heavy chain variable region CDR1 of SEQ ID NO: 2; a heavy chain variable region CDR2 of SEQ ID NO: 16; a heavy chain variable region CDR3 of SEQ ID NO: 30; a light chain variable region CDR1 of SEQ ID NO: 44; a light chain variable region CDR2 of SEQ ID NO: 58; and a light chain variable region CDR3 of SEQ ID NO: 72,
(c) a heavy chain variable region CDR1 of SEQ ID NO: 3; a heavy chain variable region CDR2 of SEQ ID NO: 17; a heavy chain variable region CDR3 of SEQ ID NO: 31; a light chain variable region CDR1 of SEQ ID NO: 45; a light chain variable region CDR2 of SEQ ID NO: 59; and a light chain variable region CDR3 of SEQ ID NO: 73,
(d) a heavy chain variable region CDR1 of SEQ ID NO: 4; a heavy chain variable region CDR2 of SEQ ID NO: 18; a heavy chain variable region CDR3 of SEQ ID NO: 32; a light chain variable region CDR1 of SEQ ID NO: 46; a light chain variable region CDR2 of SEQ ID NO: 60; and a light chain variable region CDR3 of SEQ ID NO: 74,
(e) a heavy chain variable region CDR1 of SEQ ID NO: 5; a heavy chain variable region CDR2 of SEQ ID NO: 19; a heavy chain variable region CDR3 of SEQ ID NO: 33; a light chain variable region CDR1 of SEQ ID NO: 47; a light chain variable region CDR2 of SEQ ID NO: 61; and a light chain variable region CDR3 of SEQ ID NO: 75,
(f) a heavy chain variable region CDR1 of SEQ ID NO: 6; a heavy chain variable region CDR2 of SEQ ID NO: 20; a heavy chain variable region CDR3 of SEQ ID NO: 34; a light chain variable region CDR1 of SEQ ID NO: 48; a light chain variable region CDR2 of SEQ ID NO: 62; and a light chain variable region CDR3 of SEQ ID NO: 76,
(g) a heavy chain variable region CDR1 of SEQ ID NO: 7; a heavy chain variable region CDR2 of SEQ ID NO: 21; a heavy chain variable region CDR3 of SEQ ID NO: 35; a light chain variable region CDR1 of SEQ ID NO: 49; a light chain variable region CDR2 of SEQ ID NO: 63; and a light chain variable region CDR3 of SEQ ID NO: 77,
(h) a heavy chain variable region CDR1 of SEQ ID NO: 8; a heavy chain variable region CDR2 of SEQ ID NO: 22; a heavy chain variable region CDR3 of SEQ ID NO: 36; a light chain variable region CDR1 of SEQ ID NO: 50 a light chain variable region CDR2 of SEQ ID NO: 64; and a light chain variable region CDR3 of SEQ ID NO: 78,
(i) a heavy chain variable region CDR1 of SEQ ID NO: 9; a heavy chain variable region CDR2 of SEQ ID NO: 23; a heavy chain variable region CDR3 of SEQ ID NO: 37; a light chain variable region CDR1 of SEQ ID NO: 51; a light chain variable region CDR2 of SEQ ID NO: 65; and a light chain variable region CDR3 of SEQ ID NO: 79,
(j) a heavy chain variable region CDR1 of SEQ ID NO: 10; a heavy chain variable region CDR2 of SEQ ID NO: 24; a heavy chain variable region CDR3 of SEQ ID NO: 38; a light chain variable region CDR1 of SEQ ID NO: 52; a light chain variable region CDR2 of SEQ ID NO: 66; and a light chain variable region CDR3 of SEQ ID NO: 80,
(k) a heavy chain variable region CDR1 of SEQ ID NO: 11; a heavy chain variable region CDR2 of SEQ ID NO: 25; a heavy chain variable region CDR3 of SEQ ID NO: 39; a light chain variable region CDR1 of SEQ ID NO: 53; a light chain variable region CDR2 of SEQ ID NO: 67; and a light chain variable region CDR3 of SEQ ID NO: 81,
(l) a heavy chain variable region CDR1 of SEQ ID NO: 12; a heavy chain variable region CDR2 of SEQ ID NO: 26; a heavy chain variable region CDR3 of SEQ ID NO: 40; a light chain variable region CDR1 of SEQ ID NO: 54; a light chain variable region CDR2 of SEQ ID NO: 68; and a light chain variable region CDR3 of SEQ ID NO: 82,
(m) a heavy chain variable region CDR1 of SEQ ID NO: 13; a heavy chain variable region CDR2 of SEQ ID NO: 27; a heavy chain variable region CDR3 of SEQ ID NO: 41; a light chain variable region CDR1 of SEQ ID NO: 55; a light chain variable region CDR2 of SEQ ID NO: 69; and a light chain variable region CDR3 of SEQ ID NO: 83, or
(n) a heavy chain variable region CDR1 of SEQ ID NO: 14; a heavy chain variable region CDR2 of SEQ ID NO: 28; a heavy chain variable region CDR3 of SEQ ID NO: 42; a light chain variable region CDR1 of SEQ ID NO: 56; a light chain variable region CDR2 of SEQ ID NO: 70; and a light chain variable region CDR3 of SEQ ID NO: 84.

In another embodiment, an ActRII receptor antagonist for use in treating urinary incontinence or in a method described herein may be an antibody comprising a full length heavy chain amino acid sequence having at least 95% sequence identity to at least one sequence selected from the group consisting of SEQ ID NOs: 146-150 and 156-160.

In an additional embodiment the ActRII receptor antagonist for use in treating urinary incontinence or in a method described herein may be an antibody comprising a full length light chain amino acid sequence having at least 95% sequence identity to at least one sequence selected from the group consisting of SEQ ID NOs: 141-145 and 151-155.

In one embodiment the ActRII receptor antagonist for use in treating urinary incontinence or in a method described herein may be an antibody comprising:

(a) the variable heavy chain sequence of SEQ ID NO: 99 and variable light chain sequence of SEQ ID NO: 85;
(b) the variable heavy chain sequence of SEQ ID NO: 100 and variable light chain sequence of SEQ ID NO: 86;
(c) the variable heavy chain sequence of SEQ ID NO: 101 and variable light chain sequence of SEQ ID NO: 87;
(d) the variable heavy chain sequence of SEQ ID NO: 102 and variable light chain sequence of SEQ ID NO: 88;
(e) the variable heavy chain sequence of SEQ ID NO: 103 and variable light chain sequence of SEQ ID NO: 89;
(f) the variable heavy chain sequence of SEQ ID NO: 104 and variable light chain sequence of SEQ ID NO: 90;
(g) the variable heavy chain sequence of SEQ ID NO: 105 and variable light chain sequence of SEQ ID NO: 91;
(h) the variable heavy chain sequence of SEQ ID NO: 106 and variable light chain sequence of SEQ ID NO: 92;
(i) the variable heavy chain sequence of SEQ ID NO: 107 and variable light chain sequence of SEQ ID NO: 93;
(j) the variable heavy chain sequence of SEQ ID NO: 108 and variable light chain sequence of SEQ ID NO: 94;
(k) the variable heavy chain sequence of SEQ ID NO: 109 and variable light chain sequence of SEQ ID NO: 95;
(l) the variable heavy chain sequence of SEQ ID NO: 110 and variable light chain sequence of SEQ ID NO: 96;
(m) the variable heavy chain sequence of SEQ ID NO: 111 and variable light chain sequence of SEQ ID NO: 97; or
(n) the variable heavy chain sequence of SEQ ID NO: 112 and variable light chain sequence of SEQ ID NO: 98.

In another embodiment of the disclosure the ActRII receptor antagonist for use in treating urinary incontinence or in a method described herein may be an antibody comprising:

(a) the heavy chain sequence of SEQ ID NO: 146 and light chain sequence of SEQ ID NO: 141;
(b) the heavy chain sequence of SEQ ID NO: 147 and light chain sequence of SEQ ID NO: 142;
(c) the heavy chain sequence of SEQ ID NO: 148 and light chain sequence of SEQ ID NO: 143;
(d) the heavy chain sequence of SEQ ID NO: 149 and light chain sequence of SEQ ID NO: 144;
(e) the heavy chain sequence of SEQ ID NO: 150 and light chain sequence of SEQ ID NO: 145;
(f) the heavy chain sequence of SEQ ID NO: 156 and light chain sequence of SEQ ID NO: 151;
(g) the heavy chain sequence of SEQ ID NO: 157 and light chain sequence of SEQ ID NO: 152;
(h) the heavy chain sequence of SEQ ID NO: 158 and light chain sequence of SEQ ID NO: 153;
(i) the heavy chain sequence of SEQ ID NO: 159 and light chain sequence of SEQ ID NO: 154; or
(j) the heavy chain sequence of SEQ ID NO: 160 and light chain sequence of SEQ ID NO: 155.

In yet another embodiment, the above mentioned anti-ActRII antibody comprises (i) a full length heavy chain amino acid sequence having at least 95% sequence identity to at least one sequence selected from the group consisting of SEQ ID NOs:146-150 and 156-160, (ii) a full length light chain amino acid sequence having at least 95% sequence identity to at least one sequence selected from the group consisting of SEQ ID NOs:141-145 and 151-155 or (iii) (a) the variable heavy chain sequence of SEQ ID NO: 99 and variable light chain sequence of SEQ ID NO: 85; (b) the variable heavy chain sequence of SEQ ID NO: 100 and variable light chain sequence of SEQ ID NO: 86; (c) the variable heavy chain sequence of SEQ ID NO: 101 and variable light chain sequence of SEQ ID NO: 87; (d) the variable heavy chain sequence of SEQ ID NO: 102 and variable light chain sequence of SEQ ID NO: 88; (e) the variable heavy chain sequence of SEQ ID NO: 103 and variable light chain sequence of SEQ ID NO: 89; (f) the variable heavy chain sequence of SEQ ID NO: 104 and variable light chain sequence of SEQ ID NO: 90; (g) the variable heavy chain sequence of SEQ ID NO: 105 and variable light chain sequence of SEQ ID NO: 91; (h) the variable heavy chain sequence of SEQ ID NO: 106 and variable light chain sequence of SEQ ID NO: 92; (i) the variable heavy chain sequence of SEQ ID NO: 107 and variable light chain sequence of SEQ ID NO: 93; (j) the variable heavy chain sequence of SEQ ID NO: 108 and variable light chain sequence of SEQ ID NO: 94; (k) the variable heavy chain sequence of SEQ ID NO: 109 and variable light chain sequence of SEQ ID NO: 95; (l) the variable heavy chain sequence of SEQ ID NO: 110 and variable light chain sequence of SEQ ID NO: 96; (m) the variable heavy chain sequence of SEQ ID NO: 111 and variable light chain sequence of SEQ ID NO: 97; or (n) the variable heavy chain sequence of SEQ ID NO: 112 and variable light chain sequence of SEQ ID NO: 98.

Also disclosed are ActRII receptor antagonists for use in treating urinary incontinence or in a method described herein, which are anti-ActRII receptor antibodies, which cross-block or are cross blocked by at least one antibody hereinbefore described.

In another embodiment the ActRII receptor antagonist for use in treating urinary incontinence or in a method described herein may be an anti-ActRII receptor antibody, having an altered effector function through mutation of the Fc region.

Examples of antibodies for use in treating urinary incontinence or in a method described herein are anti-ActRII antibodies encoded by pBW522 or pBW524 (deposited at DSMZ, Inhoffenstr. 7B, D-38124 Braunschweig, Germany on 18 Aug. 2009 under deposit numbers DSM22873 and DSM22874, respectively).

Furthermore, disclosed is the use of bimagrumab for treating and/or preventing urinary incontinence, or specific forms thereof like stress urinary incontinence, urge urinary incontinence and reflex urinary incontinence, wherein the urinary incontinence is caused by pelvic floor disorders resulting from a weakened or damaged pelvic muscle. The pelvic muscle can be the musculus levator ani, musculus bulbocavernosus or musculus sphincter urethrae externu and the muscle weakness or damaged is caused by the effects of childbirth or the menopause.

The working examples set forth herein describe that by using bimagrumab in the dual injury childbirth simulation rat model (Hai-Hong Jiang et al., Dual simulated childbirth injuries result in slowed recovery of pudendal nerve and urethral function; Neurourol Urodyn. 2009; 28(3): 229-235), the contemplated beneficial effect of ActRII receptor antagonists on stress urinary incontinence can be tested and proven. The working examples set forth herein provide the basis for the development of new ways of treating stress urinary incontinence or urge urinary incontinence in humans on the basis of ActRII receptor antagonists.

### Definitions

In order that the present disclosure may be more readily understood, certain terms are first defined. Additional definitions are set forth throughout the detailed description.

The term “comprising” means “including” e.g. a composition “comprising” X may consist exclusively of X or may include something additional e.g. X+Y.

The term “about” in relation to a numerical value x means, for example, x±10%.

The following exemplifies possible pre-clinical treatment regimes to evaluate possible effects of a treatment with an ActRII binding molecule, more preferably an antagonist antibody to ActRII, e.g., bimagrumab.

The treatment is exemplified by describing insights and contemplated effects of ActRII receptor antibodies for use in treating urinary incontinence using a dual injury childbirth simulation rat model, consisting of pudendal nerve crush (PNC) and vaginal distension (VD) which causes more severe and longer lasting damage than either PNC or VD alone in female rats (e.g., Hai-Hong Jiang et al., Dual simulated childbirth injuries result in slowed recovery of pudendal nerve and urethral function; Neurourol Urodyn. 2009; 28(3): 229-235; Song et al., Combination Histamine and Serotonin Treatment After Simulated Childbirth Injury Improves Stress Urinary; Neurourology and Urodynamics 35:703-710 (2016), a commonly used experimental model for urinary incontinence. The skilled person knows how to set up suitable experiments or dosing regimens for other species, in particular for humans.

The terms “ActRIIA” and “ActRIIB” refer to Activin receptors. Activins signal through a heterodimeric complex of receptor serine kinases which include at least two type I (I and IB) and two type II (IIA and IIB, aka ACVR2A and ACVR2B) receptors. These receptors are all transmembrane proteins, composed of a ligand-binding extracellular domain with a cysteine-rich region, a transmembrane domain, and a cytoplasmic domain with predicted serine/threonine specificity. Type I receptors are essential for signaling while type II receptors are required for binding ligands and for expression/recruitment of type I receptors. Type I and II receptors form a stable complex after ligand binding resulting in the phosphorylation of type I receptors by type II receptors. The activin receptor II B (ActRIIB) is a receptor for myostatin. The activin receptor II A (Act RIIA) is also a receptor for myostatin. The term ActRIIB or Act IIB receptor refers to human ActRIIB as defined in SEQ ID NO: 181 (AAC64515.1, GI:3769443). Research grade polyclonal and monoclonal anti-ActRIIB antibodies are known in the art, such as those made by R&D Systems®, MN, USA. Of course, antibodies could be raised against ActRIIB from other species and used to treat pathological conditions in those species.

By “ActRII binding molecule” is meant any molecule capable of binding to the human ActRII receptors ActRII A and/or ActRIIB either alone or associated with other molecules. The binding reaction may be shown by standard methods (qualitative assays) including, for example, a binding assay, competition assay or a bioassay for determining the inhibition of ActRII receptor binding to myostatin or any kind of binding assays, with reference to a negative control test in which an antibody of unrelated specificity, but ideally of the same isotype, e.g., an anti-CD25 antibody, is used. Non-limiting examples of ActRII receptor binding molecules include small molecules such as aptamers or other nucleic acid molecules designed and/or subject to bind the receptor, ligand decoys, and antibodies to the ActRII receptor as produced by B cells or hybridomas and chimeric, CDR-grafted or human antibodies or any fragment thereof, e.g., F(ab′)2 and Fab fragments, as well as single chain or single domain antibodies. Preferably the ActRII receptor binding molecule antagonizes (e.g., reduces, inhibits, decreases, delays) the binding of natural ligands to the ActRII receptor. In some embodiments of the disclosed methods, regimens, kits, processes and uses, an ActRIIB receptor binding molecule is employed.

A “signaling activity” refers to a biochemical causal relationship generally initiated by a protein-protein interaction such as binding of a growth factor to a receptor, resulting in transmission of a signal from one portion of a cell to another portion of a cell. In general, the transmission involves specific phosphorylation of one or more tyrosine, serine, or threonine residues on one or more proteins in the series of reactions causing signal transduction. Penultimate processes typically include nuclear events, resulting in a change in gene expression.

The term “antibody” as referred to herein includes whole antibodies and any antigen binding fragment (i.e. “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (CIq) of the classical complement system.

The term “antigen-binding portion” of an antibody (or simply “antigen portion”), as used herein, refers to full length or one or more fragments of an antibody that retain the ability to specifically bind to an antigen (e.g. a portion of ActRIIB). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments, each of which binds to the same antigen, linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g. Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. These antibody fragments are obtained using conventional techniques known to those of skill in the art, and the fragments are screened for utility in the same manner as are intact antibodies.

The terms “cross-block”, “cross-blocked” and “cross-blocking” are used interchangeably herein to mean the ability of an antibody or other binding agent to interfere with the binding of other antibodies or binding agents to ActRIIB, particularly the ligand binding domain, in a standard competitive binding assay.

The terms “monoclonal antibody” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g. human germline sequences, or mutated versions of human germline sequences or antibody containing consensus framework sequences derived from human framework sequences analysis, for example, as described in Knappik, et al. (2000. J Mol Biol 296, 57-86). The human antibodies of the disclosure may include amino acid residues not encoded by human sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Human monoclonal antibodies are produced by a hybridoma which includes a B cell obtained from a transgenic nonhuman animal, e.g. a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g. a mouse) that is transgenic or transchromosomal for human immunoglobulin genes or a hybridoma prepared therefrom, antibodies isolated from a host cell transformed to express the human antibody, e.g. from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g. IgM, IgE, IgG such as IgG1 or IgG2) that is provided by the heavy chain constant region genes.

As used herein, an antibody that “binds to ActRIIB polypeptide” is intended to refer to an antibody that binds to human ActRIIB polypeptide with a K_{D} of about 100 nM or less, about 10 nM or less, or about 1 nM or less. An antibody that “cross-reacts with an antigen other than ActRIIB” is intended to refer to an antibody that binds that antigen with a K_{D} of about 10×10⁻⁹ M or less, about 5×10⁻⁹ M or less, or about 2×10⁻⁹ M or less. An antibody that “does not cross-react with a particular antigen” is intended to refer to an antibody that binds to that antigen, with a K_{D} of about 1.5×10⁻⁸ M or greater, or a K_{D} of about 5-10×10⁻⁸ M, or about 1×10⁻⁷ M or greater. In certain embodiments, such antibodies that do not cross-react with the antigen exhibit essentially undetectable binding against these proteins in standard binding assays. K_{D} may be determined using a biosensor system, such as a Biacore® system, or Solution Equilibrium Titration.

As used herein, the term “antagonist antibody” is intended to refer to an antibody that inhibits ActRIIB induced signaling activity in the presence of myostatin or of other ActRIIB ligands such as activins or GDF-11 and/or to an antibody that inhibits ActRIIA induced signaling activity in the presence of myostatin or of other ActRIIA ligands such as activins or GDF-11. Examples of an assay to detect this include inhibition of myostatin induced signaling (for instance by a Smad dependent reporter gene assay), inhibition of myostatin induced Smad phosphorylation (P-Smad ELISA) and inhibition of myostatin induced inhibition of skeletal muscle cell differentiation (for instance by a creatine kinase assay).

In some embodiments, the antibodies that binds to the ActRIIB polypeptide inhibit myostatin induced signaling as measured in a Smad dependent reporter gene assay at an IC₅₀ of about 10 nM or less, about 1 nM or less, or about 100 pM or less.

The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, such as the biosensor system of Biacore®, or Solution Equilibrium Titration (SET) (see Friguet B et al. (1985) J. Immunol Methods; 77(2): 305-319, and Hanel C et al. (2005) Anal Biochem; 339(1): 182-184).

As used herein, the term “ADCC” or “antibody dependent cellular cytotoxicity” activity refers to human B cell depleting activity. ADCC activity can be measured by the human B cell depleting assays known in the art.

As used herein, the term, “optimized” means that a nucleotide sequence has been altered to encode an amino acid sequence using codons that are preferred in the production cell or organism, generally a eukaryotic cell, for example, a cell of Pichia, a cell of Trichoderma, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in CHO mammalian cells, however optimized expression of these sequences in other eukaryotic cells is also envisioned herein. The amino acid sequences encoded by optimized nucleotide sequences are also referred to as optimized.

As used herein, the term “a therapeutically effective amount” of the compound of the present invention refers to an amount of the compound of the present invention that will elicit the biological or medical response of a subject, for example, ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to at least partially alleviating, inhibiting, preventing and/or ameliorating a condition associated with urinary incontinence. Urinary incontinence symptoms/conditions are (i) incontinence following a sudden cough, sneezing, laughing, heavy lifting and exercise or (ii) involuntary contraction of the muscular wall of the bladder that causes an urge to urinate that cannot be stopped or (iii) bladder cannot hold as much urine as the body is making and/or the bladder cannot empty completely, causing small amounts of urinary leakage (patients experiencing constant “dribbling” of urine from the urethra).

As used herein, the term urinary incontinence refers to the all degrees or sensitivity ranges of loss of bladder control, like incontinence episodes per 24 hours, number of micturations per 24 hours, volume voided per incontinence, nocturia episodes per 24 hours or an improvement in Patient Perception of Bladder Condition. The severity ranges from occasionally leaking urine when coughing or sneezing to having a sudden urge to urinate. It occurs when the interior pressure of the bladder is larger than the resistance of the urethra. It is reported that urinary incontinence generally results from the decrease in ability to regulate the urethra due to drooping of bladder, extension of the pelvic muscles, including levator ani and bulbocavernosus muscles, and weakness of the urethra sphincter. There are several types of urinary incontinence: stress urinary incontinence (SUI) occurs when body movements put pressure on the bladder suddenly; urge urinary incontinence (UUI) occurs when people cannot hold their urine long enough to get to the toilet in time due to sensitivity of bladder muscle and when bladder leaks urine due to extreme stimulus such as a medical conditions including bladder cancer, bladder inflammation, bladder outlet obstruction, bladder stones, or bladder infection; psychogenic incontinence occurs due to dementia; and neurogenic urinary incontinence (NUI) occurs due to damage to the nerves that govern the urinary tract. Stress incontinence is the most common type of bladder control problem in younger and middle-age women. Stress Urinary Incontinence occurs when the bladder leaks urine during physical activity. It may happen when coughing, doing exercise or lifting heavy items. Predisposing factors are pregnancy or menopause. Men may develop stress incontinence following benign prostatic hyperplasia or prostate cancer surgical treatment. The amount of urine voided per incontinence may vary from a few drops to 100 mL or more. In some cases, it is related to the effects of childbirth. It may also begin around the time of menopause. Reflex Urinary Incontinence involves dysfunction of the neurological control mechanisms for detrusor contraction and sphincter relaxation. RUI can occur as a result of stroke, Parkinson's disease, brain tumors, spinal cord injuries or multiple sclerosis. RUI patients experiences periodic urination without an awareness of needing to void.

Stress urinary incontinence can coexist with urge urinary incontinence (UUI). Urge urinary incontinence is part of a complex known as overactive or oversensitive bladder, which includes symptoms of frequency and/or urgency with or without urge urinary incontinence. 75 percent of patients with incontinence are elderly females. Stress urinary incontinence (SUI), the involuntary leakage of urine during activities that increase abdominal pressure (e.g. coughing, sneezing, physical exercise), affects up to 35 percent of adult women (Luber K M. The definition, prevalence, and risk factors for stress urinary incontinence. Rev Urol (suppl.) 2004; 6: S3).

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

### DETAILED DESCRIPTION OF THE DISCLOSURE

It is contemplated that antibodies directed to the ActRII receptors, e.g., bimagrumab, can decrease signaling through these receptors, and result in prevention and/or treatment of urinary incontinence. Stress urinary incontinence (SUI) is increasing in prevalence worldwide, with huge adverse consequences on quality of life in affected individuals. Weakness of the pelvic floor musculature, resulting from birth trauma, menopause and aging in women can result in lack of support for the urethra resulting in stress incontinence and innervation changes and feedback mechanisms can lead to urge incontinence. There are limited effective pharmaceutical interventions to treat stress incontinence.

Therefore, in one aspect, the disclosure provides ActRII binding molecules, e.g., bimagrumab or a functional protein comprising an antigen-binding portion of said antibody, for use in treating urinary incontinence. Preferably the ActRII antibody binds human ActRIIB and ActRIIA protein. The polypeptide sequence of the human ActRIIB is recited in SEQ ID NO: 181 (AAC64515.1, GI:3769443). The human ActRIIA protein has the Genbank accession No. AAH67417.1 (NP_001607.1, GI:4501897). In one embodiment, the antibody or functional protein for use in treating urinary incontinence is from a mammal, having an origin such as human or camelid. Thus the antibody for use in treating urinary incontinence may be a chimeric, human or a humanized antibody. In a particular embodiment, the anti-ActRII antibody for use in treating urinary incontinence is characterized as being a human monoclonal antibody having an antigen-binding region that is specific for the human target protein ActRIIB and binds to ActRIIB and ActRIIA or fragments thereof.

In one embodiment, the antibodies for use in treating urinary incontinence are ActRII antagonists with no or low agonistic activity. In another embodiment, the antibody or a functional fragment thereof binds the target protein ActRII and decreases the binding of myostatin to ActRII to a basal level. In a further aspect of this embodiment, the antibody or functional fragment thereof employed in the inventive methods or for use in treating urinary incontinence completely prevents myostatin from binding to ActRIIB. In a further embodiment, the antibody or functional fragment thereof employed in the inventive methods or for use in treating urinary incontinence inhibits Smad activation. In a further embodiment, the antibody or functional fragment thereof employed in the inventive methods or for use in treating urinary incontinence inhibits activin receptor type IIB mediated myostatin-induced inhibition of skeletal differentiation via the Smad-dependent pathway.

The binding may be determined by one or more assays that can be used to measure an activity which is either antagonism or agonism by the antibody. Preferably, the assays measure at least one of the effects of the antibody on ActRIIB that include: inhibition of myostatin binding to ActRIIB by ELISA, inhibition of myostatin induced signaling (for instance by a Smad dependent reporter gene assay), inhibition of myostatin induced Smad phosphorylation (P-Smad ELISA) and inhibition of myostatin induced inhibition of skeletal muscle cell differentiation (for instance by a creatine kinase assay).

In one embodiment, compositions comprising antibodies that specifically bind to the myostatin binding region (i.e. ligand binding domain) of ActRIIB can be employed in the inventive methods for treating urinary incontinence or used in treating urinary incontinence in a patient in need thereof. This ligand binding domain consists of amino acids 19-134 of SEQ ID NO: 181 and has been assigned SEQ ID NO: 182 herein. The ligand biding domain comprises several below described epitopes.

In one embodiment, the antibodies comprised in a composition employed in the inventive methods for treating urinary incontinence or used in treating urinary incontinence bind to ActRIIB with a K_{D} of about 100 nM or less, about 10 nM or less, about 1 nM or less. Preferably, the antibodies comprised in a composition employed in the inventive methods for treating urinary incontinence or used in treating urinary incontinence bind to ActRIIB with an affinity of 100 pM or less (i.e. about 100 pM, about 50 pM, about 10 pM, about 2 pM, about 1 pM or less). In one embodiment, the antibodies comprised in a composition employed in the inventive methods for treating urinary incontinence or used in treating urinary incontinence bind to ActRIIB with an affinity of between about 1 and about 10 pM.

In another embodiment, the antibodies comprised in a composition employed in the inventive methods for treating urinary incontinence or used in treating urinary incontinence cross-react with ActRIIA and bind to ActRIIB with equivalent affinity, or about 1, 2, 3, 4 or 5-fold greater affinity than they bind to ActRIIA, more preferably about 10-fold, still more preferably about 20-, 30-, 40- or 50-fold, still more preferably about 100-fold.

In one embodiment, the antibodies comprised in a composition employed in the inventive methods for treating urinary incontinence or used in treating urinary incontinence bind to ActRIIA with an affinity of 100 pM or more (i.e. about 250 pM, about 500 pM, about 1 nM, about 5 nM or more).

In one embodiment, the antibodies comprised in a composition employed in the inventive methods for treating urinary incontinence or used in treating urinary incontinence are of the IgG₂ isotype.

In another embodiment, the antibodies comprised in a composition employed in the inventive methods for treating urinary incontinence or used in treating urinary incontinence are of the IgG₁ isotype. In a further embodiment, the antibodies comprised in a composition employed in the inventive methods for treating urinary incontinence or used in treating urinary incontinence are of the IgG1 isotype and have an altered effector function through mutation of the Fc region. Said altered effector function may be a reduced ADCC and CDC activity. In one embodiment, said altered effector function is silenced ADCC and CDC activity.

In another related embodiment, the antibodies comprised in a composition employed in the inventive methods for treating urinary incontinence or used in treating urinary incontinence are fully human or humanized IgG1 antibodies with no antibody dependent cellular cytotoxicity (ADCC) activity or CDC activity and bind to a region of ActRIIB consisting of amino acids 19-134 of SEQ ID NO:181.

In another related embodiment, the antibodies comprised in a composition employed in the inventive methods for treating urinary incontinence or used in treating urinary incontinence are fully human or humanized IgG1 antibodies with reduced antibody dependent cellular cytotoxicity (ADCC) activity or CDC activity and bind to a region of ActRIIB consisting of amino acids 19-134 of SEQ ID NO:181.

The present disclosure also relates to the use of compositions comprising human or humanized anti-ActRII antibodies for use in preventing and/or treatment of urinary incontinence.

In certain embodiments, the antibodies comprised in a composition employed in the inventive methods for treating urinary incontinence or used in treating urinary incontinence are derived from particular heavy and light chain sequences and/or comprise particular structural features such as CDR regions comprising particular amino acid sequences. The disclosure provides isolated ActRIIB antibodies, methods of making such antibodies, immunoconjugates and multivalent or multispecific molecules comprising such antibodies and pharmaceutical compositions containing the antibodies, immune-conjugates or bispecific molecules.

In another related embodiment, the antibody comprised in a composition employed in the inventive methods for treating urinary incontinence or used in treating urinary incontinence is bimagrumab. Bimagrumab is the INN (international non-proprietary name) of a monoclonal human antibody also known as BYM338 or MOR08159 developed to bind competitively to activin receptor type IIB (ActRII) with greater affinity than myostatin or activin, its natural ligands. Bimagrumab is disclosed in WO2010/125003. The bimagrumab sequences disclosed in WO2010/1253003 are listed in table 1.

In another related embodiment, the antibody comprised in a composition employed in the inventive methods for treating urinary incontinence or used in treating urinary incontinence is the antibody MOR08213. MOR08213 is a monoclonal antibody developed to bind competitively to activin receptor type IIB (ActRII) with greater affinity than myostatin or activin, its natural ligands. MOR08213 is disclosed in WO2010/125003. The MOR08213 sequences disclosed in WO2010/1253003 are listed in table 2.

Furthermore, the herein described inventive treatment methods and uses can be combined with pelvic floor muscle training exercise.

TABLE 1

| Antibody | Ab region | SEQ ID NO: |
|---|---|---|
| bimagrumab | HCDR1 | SEQ ID NO: 9 |
| bimagrumab | HCDR2 | SEQ ID NO: 23 |
| bimagrumab | HCDR3 | SEQ ID NO: 37 |
| bimagrumab | LCDR1 | SEQ ID NO: 51 |
| bimagrumab | LDCR2 | SEQ ID NO: 65 |
| bimagrumab | LCDR3 | SEQ ID NO: 79 |
| bimagrumab | VL | SEQ ID NO: 93 |
| bimagrumab | VH | SEQ ID NO: 107 |
| bimagrumab | DNA VL | SEQ ID NO: 121 |
| bimagrumab | DNA VH | SEQ ID NO: 135 |
| bimagrumab | Optimized Light IgG1 LALA | SEQ ID NO: 141 |
| bimagrumab | Optimized Heavy IgG1 LALA | SEQ ID NO: 146 |
| bimagrumab | Optimized Light IgG2 | SEQ ID NO: 151 |
| bimagrumab | Optimized Heavy IgG2 | SEQ ID NO: 156 |
| bimagrumab | DNA opt Light IgG1 LALA | SEQ ID NO: 161 |
| bimagrumab | DNA opt Heavy IgG1 LALA | SEQ ID NO: 166 |
| bimagrumab | DNA opt Light IgG2 | SEQ ID NO: 171 |
| bimagrumab | DNA opt Heavy IgG2 | SEQ ID NO: 176 |

A plasmid designated pBW524 comprising the VL and VH coding regions of bimagrumab has been deposited at DSMZ, Inhoffenstr. 7B, D-38124 Braunschweig, Germany on 18 Aug. 2009 under deposit number DSM22874

TABLE 2

| Clone | Ab region | SEQ ID NO: |
|---|---|---|
| MOR08213 | HCDR1 | SEQ ID NO: 10 |
| MOR08213 | HCDR2 | SEQ ID NO: 24 |
| MOR08213 | HCDR3 | SEQ ID NO: 38 |
| MOR08213 | LCDR1 | SEQ ID NO: 52 |
| MOR08213 | LDCR2 | SEQ ID NO: 66 |
| MOR08213 | LCDR3 | SEQ ID NO: 80 |
| MOR08213 | VL | SEQ ID NO: 94 |
| MOR08213 | VH | SEQ ID NO: 108 |
| MOR08213 | DNA VL | SEQ ID NO: 122 |
| MOR08213 | DNA VH | SEQ ID NO: 136 |
| MOR08213 | Optimized Light IgG1 LALA | SEQ ID NO: 142 |
| MOR08213 | Optimized Heavy IgG 1 LALA | SEQ ID NO: 147 |
| MOR08213 | Optimized Light IgG2 | SEQ ID NO: 152 |
| MOR08213 | Optimized Heavy IgG2 | SEQ ID NO: 157 |
| MOR08213 | DNAopt Light IgG1 LALA | SEQ ID NO: 162 |
| MOR08213 | DNAopt Heavy IgG1 LALA | SEQ ID NO: 167 |
| MOR08213 | DNA opt Light IgG2 | SEQ ID NO: 172 |
| MOR08213 | DNA opt Heavy IgG2 | SEQ ID NO: 177 |

A plasmid designated pBW522 comprising the VL and VH coding regions of MOR08213 has been deposited at DSMZ, Inhoffenstr. 7B, D-38124 Braunschweig, Germany on 18 Aug. 2009 under deposit number DSM22873 In alternative embodiments, the disclosure relates to the following aspects:

1. An ActRII receptor antagonist for use in treating and/or preventing urinary incontinence including urinary incontinence associated with, or caused by pelvic floor disorders resulting from a weakened or damaged pelvic muscle. The pelvic muscle can be the musculus levator ani, musculus bulbocavernosus or musculus sphincter urethrae externu and the muscle weakness or damaged is caused by the effects of childbirth or the menopause.
2. An ActRII receptor antagonist for use according to aspect 1, wherein the ActRII antagonist is to be administered to a patient in need thereof at a dose of about 3-10 mg/kg.
3. An ActRII receptor antagonist for use according to aspect 2, wherein said myostatin antagonist is to be administered at a dose of about 3 or about 10 mg/kg body weight.

Alternatively, the ActRII receptor antagonist is to be administered at a dose of about 3, 4, 5, 6, 7, 8, 9 or about 10 mg/kg body weight.

4. An ActRII receptor antagonist for use according to aspect 1-3, wherein said ActRII receptor antagonist is to be administered intravenously or subcutaneously.
5. An ActRII receptor antagonist for use according to anyone of aspects 1-4, wherein said ActRII receptor antagonist is to be administered every four weeks.

Alternatively, the ActRII receptor antagonist can be administered subcutaneously on a weekly basis.

Alternatively, the ActRII receptor antagonist can be administered every 8 weeks.

6. An ActRII receptor antagonist for use according to anyone of aspects 1-5, wherein said ActRII receptor antagonist is to be administered for at least 3 months.
7. An ActRII receptor antagonist for use according to anyone of aspects 1-6, wherein said ActRII receptor antagonist is to be administered for up to 12 months. Preferably the ActRII receptor antagonist is to be administered for at least or up to 3, 4, 5, 6, 7, 8, 9, 10, 11 or 12 months.
8. A method of treating and/or preventing urinary incontinence, said method comprising administering an effective amount of an ActRII receptor antagonist to a subject who has urinary incontinence or who is at risk of developing urinary incontinence.
9. A method of treating urinary incontinence said method comprising administering an effective amount of an ActRII receptor antagonist to a subject showing symptoms/suffering from urinary incontinence.
10. A method according to aspects 8 or 9, comprising administering the ActRII receptor antagonist to a patient in need thereof at a dose of about 3-10 mg/kg.
11. A method according to aspects 8 or 9, comprising administering the ActRII receptor antagonist to a patient in need thereof at a dose of about 3 or about 10 mg/kg body weight.
12. A method according to aspects 8 or 9, comprising administering the ActRII receptor antagonist intravenously or subcutaneously.
13. A method according to any one of aspects 8 to 10, comprising administering the ActRII receptor antagonist every four weeks.

Alternatively, the ActRII receptor antagonist can be administered in the method according to aspect 13 subcutaneously on a weekly basis.

14. A method according to any one of aspects 8 to 13, comprising administering the ActRII receptor antagonist for at least 3 months.
15. A method according to aspect 14, comprising administering the ActRII receptor antagonist for up to 12 months.
16. An ActRII receptor antagonist for use according to any one of aspects 1-7 or a method of treatment according to any one of aspects 8-15, wherein the ActRII receptor antagonist is an anti-ActRII receptor antibody or an antigen-binding portion thereof.
17. An ActRII receptor antagonist for use according to any one of aspects 1-7 or a method of treatment according to any one of aspects 8-15, wherein the ActRII receptor antagonist is an anti-ActRII receptor antibody or an antigen-binding portion thereof, and wherein the anti-ActRII receptor antibody is bimagrumab or an antigen-binding portion thereof.
18. An ActRII receptor antagonist for use according to any one of aspects 1-7 or a method of treatment according to any one of aspects 8-15, wherein the ActRII receptor antagonist is an anti-ActRII receptor antibody or an antigen-binding portion thereof, and wherein the antibody comprises a full length heavy chain amino acid sequence having at least 95% sequence identity to at least one sequence selected from the group consisting of SEQ ID NOs: 146-150 and 156-160 and, wherein the antibody comprises a full length light chain amino acid sequence having at least 95% sequence identity to at least one sequence selected from the group consisting of SEQ ID NOs: 141-145 and 151-155.
19. An ActRII receptor antagonist for use according to any one of aspects 1-7 or a method of treatment according to any one of aspects 8-15, wherein the ActRII receptor antagonist is an anti-ActRII receptor antibody or an antigen-binding portion thereof, and wherein the antibody is encoded by pBW522 (DSM22873) or pBW524 (DSM22874).
20. Bimagrumab or an antigen-binding portion thereof for use according to any one of aspects 1-7 or a method of treatment according to any one of aspects 8-15, wherein bimagrumab is to be administered intravenously at a dose of about 3-10 mg/kg body weight every four weeks.

Bimagrumab or an antigen-binding portion thereof for use according to aspect 20, wherein bimagrumab is to be administered subcutaneously at a dose of about 3-10 mg/kg body weight on a weekly basis.

21. A composition comprising 150 mg/ml of bimagrumab or an antigen binding portion thereof for use in treating and/or preventing urinary incontinence.
22. A unitary dosage form comprising 150 mg/ml of bimagrumab or an antigen binding portion thereof for use in treating and/or preventing urinary incontinence. In further embodiments, the unitary dosage form, i.e., a vial, comprises 100-200 mg/ml of bimagrumab, preferably 100, 105, 110, 115, 120, 125, 130, 135, 140, 145, 150, 155, 160, 165, 170, 175, 180, 185, 190, 195, 200 mg/ml of bimagrumab.
23. An infusion bag comprising an appropriate amount of bimagrumab from one or more vials diluted with a solution for use in treating and/or preventing urinary incontinence.

The solution is preferably a dextrose solution.

In some further embodiments, the ActRII receptor antagonist or anti-ActRII antibody such as bimagrumab used in the inventive methods or for use in treating and/or preventing urinary incontinence is to be administered at a dose of about 1, 2, 3, 4, 5, 5, 6, 7, 8, 9, 10 mg/kg body weight.

Disclosed herein are ActRII receptor antagonists for the manufacture of a medicament for treating and/or preventing urinary incontinence.

In another related embodiment, the ActRII receptor antagonist for the manufacture of a medicament for treating and/or preventing urinary incontinence is bimagrumab or MOR08213.

In further embodiments, all the aspects disclosed herein can be used in combination one with any of the other.

Various aspects of the disclosure are described in further detail in the following subsections. Standard assays to evaluate the binding ability of the antibodies toward ActRII of various species are known in the art, including for example, ELISAs, western blots and RIAs. The binding affinity of the antibodies also can be assessed by standard assays known in the art, such as by Biacore analysis or Solution Equilibrium Titration. Surface plasmon resonance based techniques such as Biacore can determine the binding kinetics which allows the calculation of the binding affinity.

Accordingly, an antibody that “inhibits” one or more of these ActRII functional properties (e.g. biochemical, immunochemical, cellular, physiological or other biological activities, or the like) as determined according to methodologies known to the art and described herein, will be understood to relate to a statistically significant decrease in the particular activity relative to that seen in the absence of the antibody (e.g. or when a control antibody of irrelevant specificity is present). An antibody that inhibits ActRII activity effects such a statistically significant decrease by at least 10% of the measured parameter, by at least 50%, 80% or 90%, and in certain embodiments an antibody of the disclosure may inhibit greater than 95%, 98% or 99% of ActRIIB functional activity.

The ability or extent to which an antibody or other binding agent is able to interfere with the binding of another antibody or binding molecule to ActRII, and therefore whether it can be said to cross-block according to the disclosure, can be determined using standard competition binding assays. One suitable assay involves the use of the Biacore technology (e.g. by using a BIAcore instrument (Biacore, Uppsala, Sweden)), which can measure the extent of interactions using surface plasmon resonance technology. Another assay for measuring cross-blocking uses an ELISA-based approach. A further assay uses FACS analysis, wherein competition of various antibodies for binding to ActRIIB expressing cells is tested.

According to the disclosure, a cross-blocking antibody or other binding agent according to the disclosure binds to ActRIIB in the described BIAcore cross-blocking assay such that the recorded binding of the combination (mixture) of the antibodies or binding agents is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%), and more specifically between 65% and 0.1% (e.g. 65% to 4%) of maximum theoretical binding (as defined above) of the two antibodies or binding agents in combination.

An antibody is defined as cross-blocking an anti-ActRIIB antibody of the disclosure in an ELISA assay, if the test antibody is able to cause a reduction of anti-ActRIIB antibody binding to ActRIIB of between 60% and 100%, specifically between 70% and 100%, and more specifically between 80% and 100%, when compared to the positive control wells (i.e. the same anti-ActRIIB antibody and ActRIIB, but no “test” cross-blocking antibody). Examples of cross blocking antibodies as cited herein are bimagrumab and MOR08213 (disclosed in WO2010/125003).

### Recombinant Antibodies

Antibodies, e.g., antagonist antibodies to ActRII, such as bimagrumab, comprised in the compositions used within this disclosure include the human recombinant antibodies, isolated and structurally characterized, as described herein. The V_{H} amino acid sequences of antibodies comprised in the inventive compositions are shown in SEQ ID NOs: 99-112. The V_{L} amino acid sequences of antibodies comprised in the inventive compositions are shown in SEQ ID NOs: 85-98 respectively. Examples of preferred full length heavy chain amino acid sequences of antibodies comprised in the inventive compositions are shown in SEQ ID NOs: 146-150 and 156-160. Examples of preferred full length light chain amino acid sequences of antibodies comprised in the inventive compositions are shown in SEQ ID NOs: 141-145 and 151-155 respectively. Other antibodies comprised in the inventive compositions include amino acids that have been mutated by amino acid deletion, insertion or substitution, yet have at least 60, 70, 80, 90, 95, 97 or 99 percent identity in the CDR regions with the CDR regions depicted in the sequences described above. In some embodiments, it includes mutant amino acid sequences wherein no more than 1, 2, 3, 4 or 5 amino acids have been mutated by amino acid deletion, insertion or substitution in the CDR regions when compared with the CDR regions depicted in the sequence described above.

Further, variable heavy chain parental nucleotide sequences are shown in SEQ ID NOs: 127-140. Variable light chain parental nucleotide sequences are shown in SEQ ID NOs: 113-126. Full length light chain nucleotide sequences optimized for expression in a mammalian cell are shown in SEQ ID NOs: 161-165 and 171-175. Full length heavy chain nucleotide sequences optimized for expression in a mammalian cell are shown in SEQ ID NOs: 166-170 and 176-180. Other antibodies comprised in compositions employed in the inventive methods for treating urinary incontinence or used in treating urinary incontinence include amino acids or are encoded by nucleic acids that have been mutated, yet have at least 60 or more (i.e. 80, 90, 95, 97, 99 or more) percent identity to the sequences described above. In some embodiments, it includes mutant amino acid sequences wherein no more than 1, 2, 3, 4 or 5 amino acids have been mutated by amino acid deletion, insertion or substitution in the variable regions when compared with the variable regions depicted in the sequence described above.

Since each of these antibodies binds the same epitope and are progenies from the same parental antibody, the V_{H}, V_{L}, full length light chain, and full length heavy chain sequences (nucleotide sequences and amino acid sequences) can be “mixed and matched” to create other anti-ActRIIB binding molecules of the disclosure. ActRIIB binding of such “mixed and matched” antibodies can be tested using the binding assays described above and in well-known methods, such as e.g. ELISAs. When these chains are mixed and matched, a V_{H} sequence from a particular V_{H}/V_{L} pairing should be replaced with a structurally similar V_{H} sequence. Likewise, a full-length heavy chain sequence from a particular full length heavy chain/full length light chain pairing should be replaced with a structurally similar full length heavy chain sequence. Likewise, a V_{L} sequence from a particular V_{H}/V_{L} pairing should be replaced with a structurally similar V_{L} sequence. Likewise, a full-length light chain sequence from a particular full length heavy chain/full length light chain pairing should be replaced with a structurally similar full length light chain sequence. Accordingly, in one aspect, the disclosure provides compositions employed in the inventive methods for treating urinary incontinence or used in treating urinary incontinence comprising a recombinant anti-ActRII antibody or antigen binding region thereof having: a heavy chain variable region comprising an amino acid sequence selected from the group consisting of SEQ ID NOs: 99-112; and a light chain variable region comprising an amino acid sequence selected from the group consisting of SEQ ID NOs: 85-98.

In another aspect, the disclosure provides compositions that can be employed in the inventive methods for treating urinary incontinence or used in treating urinary incontinence comprising:

(i) an isolated recombinant anti-ActRII antibody having: a full length heavy chain comprising an amino acid sequence selected from the group consisting of SEQ ID NOs:99-112; and a full length light chain comprising an amino acid sequence selected from the group consisting of SEQ ID NOs:85-98, or
(ii) a functional protein comprising an antigen binding portion thereof.

In another aspect, the disclosure provides compositions that can be employed in the inventive methods for treating urinary incontinence or used in treating urinary incontinence comprising:

(i) an isolated recombinant anti-ActRII antibody having a full length heavy chain encoded by a nucleotide sequence that has been optimized for expression in the cell of a mammalian selected from the group consisting of SEQ ID NOs:127-140, and a full length light chain encoded by a nucleotide sequence that has been optimized for expression in the cell of a mammalian selected from the group consisting of SEQ ID NOs:113-126, or
(ii) a functional protein comprising an antigen binding portion thereof.

Examples of amino acid sequences of the V_{H} CDR1s of the antibodies comprised in the inventive compositions are shown in SEQ ID NOs: 1-14. The amino acid sequences of the V_{H} CDR2s of the antibodies are shown in SEQ ID NOs: 15-28. The amino acid sequences of the V_{H} CDR3s of the antibodies are shown in SEQ ID NOs: 29-42. The amino acid sequences of the V_{L} CDR1s of the antibodies are shown in SEQ ID NOs: 43-56. The amino acid sequences of the V_{L} CDR2s of the antibodies are shown in SEQ ID NOs: 57-70. The amino acid sequences of the V_{L} CDR3s of the antibodies are shown in SEQ ID NOs: 71-84. The CDR regions are delineated using the Kabat system (Kabat, E. A., et al., 1991 Sequences of Proteins of Immunological Interest, Fifth Edition, U.S. Department of Health and Human Services, NIH Publication No. 91-3242). An alternative method of determining CDR regions uses the method devised by Chothia (Chothia et al. 1989, Nature, 342:877-883). The Chothia definition is based on the location of the structural loop regions. However, due to changes in the numbering system used by Chothia (see e.g. http://www.biochem.ucl.ac.uk/˜martin/abs/Generallnfo.html and http://www.bioinf.org.uk/abs/), this system is now less commonly used. Other systems for defining CDRs exist and are also mentioned in these two websites.

Given that each of these antibodies can bind to ActRIIB and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e. CDRs from different antibodies can be mixed and matched, each antibody containing a V_{H} CDR1, 2 and 3 and a V_{L} CDR1, 2 and 3 create other anti-ActRII binding molecules of the disclosure. ActRIIB binding of such “mixed and matched” antibodies can be tested using the binding assays described above and in the Examples (e.g. ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences shown herein for monoclonal antibodies.

An anti-ActRII antibody or an antigen binding portion thereof that can be employed in the inventive methods for treating urinary incontinence or used in treating urinary incontinence has: a heavy chain variable region CDR1 comprising an amino acid sequence selected from the group consisting of SEQ ID NOs: 1-14; a heavy chain variable region CDR2 comprising an amino acid sequence selected from the group consisting of SEQ ID NOs: 15-28; a heavy chain variable region CDR3 comprising an amino acid sequence selected from the group consisting of SEQ ID NOs: 29-42; a light chain variable region CDR1 comprising an amino acid sequence selected from the group consisting of SEQ ID NOs: 43-56; a light chain variable region CDR2 comprising an amino acid sequence selected from the group consisting of SEQ ID NOs: 57-70; and a light chain variable region CDR3 comprising an amino acid sequence selected from the group consisting of SEQ ID NOs: 71-84.

In one embodiment, the antibody comprised in the composition employed in the inventive methods for treating urinary incontinence or used in treating urinary incontinence comprises: a heavy chain variable region CDR1 of SEQ ID NO: 1; a heavy chain variable region CDR2 of SEQ ID NO: 15; a heavy chain variable region CDR3 of SEQ ID NO: 29; a light chain variable region CDR1 of SEQ ID NO: 43; a light chain variable region CDR2 of SEQ ID NO: 57; and a light chain variable region CDR3 of SEQ ID NO: 71.

In one embodiment, the antibody comprised in the composition employed in the inventive methods for treating urinary incontinence or used in treating urinary incontinence comprises: a heavy chain variable region CDR1 of SEQ ID NO: 2 a heavy chain variable region CDR2 of SEQ ID NO: 16; a heavy chain variable region CDR3 of SEQ ID NO: 30; a light chain variable region CDR1 of SEQ ID NO: 44; a light chain variable region CDR2 of SEQ ID NO: 58; and a light chain variable region CDR3 of SEQ ID NO: 72.

In one embodiment, the antibody comprised in the composition employed in the inventive methods for treating urinary incontinence or used in treating urinary incontinence comprises: a heavy chain variable region CDR1 of SEQ ID NO: 3; a heavy chain variable region CDR2 of SEQ ID NO: 17; a heavy chain variable region CDR3 of SEQ ID NO: 31; a light chain variable region CDR1 of SEQ ID NO: 45; a light chain variable region CDR2 of SEQ ID NO: 59; and a light chain variable region CDR3 of SEQ ID NO: 73.

In one embodiment, the antibody comprised in the composition employed in the inventive methods for treating urinary incontinence or used in treating urinary incontinence comprises: a heavy chain variable region CDR1 of SEQ ID NO: 4; a heavy chain variable region CDR2 of SEQ ID NO: 18; a heavy chain variable region CDR3 of SEQ ID NO: 32; a light chain variable region CDR1 of SEQ ID NO: 46; a light chain variable region CDR2 of SEQ ID NO: 60; and a light chain variable region CDR3 of SEQ ID NO: 74.

In one embodiment, the antibody comprised in the composition employed in the inventive methods for treating urinary incontinence or used in treating urinary incontinence comprises: a heavy chain variable region CDR1 of SEQ ID NO: 5; a heavy chain variable region CDR2 of SEQ ID NO: 19; a heavy chain variable region CDR3 of SEQ ID NO: 33; a light chain variable region CDR1 of SEQ ID NO: 47; a light chain variable region CDR2 of SEQ ID NO: 61; and a light chain variable region CDR3 of SEQ ID NO: 75.

In one embodiment, the antibody comprised in the composition employed in the inventive methods for treating urinary incontinence or used in treating urinary incontinence comprises: a heavy chain variable region CDR1 of SEQ ID NO: 6; a heavy chain variable region CDR2 of SEQ ID NO: 20; a heavy chain variable region CDR3 of SEQ ID NO: 34; a light chain variable region CDR1 of SEQ ID NO: 48; a light chain variable region CDR2 of SEQ ID NO: 62; and a light chain variable region CDR3 of SEQ ID NO: 76.

In one embodiment, the antibody comprised in the composition employed in the inventive methods for treating urinary incontinence or used in treating urinary incontinence comprises: a heavy chain variable region CDR1 of SEQ ID NO: 7; a heavy chain variable region CDR2 of SEQ ID NO: 21; a heavy chain variable region CDR3 of SEQ ID NO: 35; a light chain variable region CDR1 of SEQ ID NO: 49; a light chain variable region CDR2 of SEQ ID NO: 63; and a light chain variable region CDR3 of SEQ ID NO: 77.

In one embodiment, the antibody comprised in the composition employed in the inventive methods for treating urinary incontinence or used in treating urinary incontinence comprises: a heavy chain variable region CDR1 of SEQ ID NO: 8; a heavy chain variable region CDR2 of SEQ ID NO: 22; a heavy chain variable region CDR3 of SEQ ID NO: 36; a light chain variable region CDR1 of SEQ ID NO: 50 a light chain variable region CDR2 of SEQ ID NO: 64; and a light chain variable region CDR3 of SEQ ID NO: 78.

In one embodiment, the antibody comprised in the composition employed in the inventive methods for treating urinary incontinence or used in treating urinary incontinence comprises: a heavy chain variable region CDR1 of SEQ ID NO: 9; a heavy chain variable region CDR2 of SEQ ID NO: 23; a heavy chain variable region CDR3 of SEQ ID NO: 37; a light chain variable region CDR1 of SEQ ID NO: 51; a light chain variable region CDR2 of SEQ ID NO: 65; and a light chain variable region CDR3 of SEQ ID NO: 79.

In one embodiment, the antibody comprised in the composition employed in the inventive methods for treating urinary incontinence or used in treating urinary incontinence comprises: a heavy chain variable region CDR1 of SEQ ID NO: 10; a heavy chain variable region CDR2 of SEQ ID NO: 24; a heavy chain variable region CDR3 of SEQ ID NO: 38; a light chain variable region CDR1 of SEQ ID NO: 52; a light chain variable region CDR2 of SEQ ID NO: 66; and a light chain variable region CDR3 of SEQ ID NO: 80.

In one embodiment, the antibody comprised in the composition employed in the inventive methods for treating urinary incontinence or used in treating urinary incontinence comprises: a heavy chain variable region CDR1 of SEQ ID NO: 11; a heavy chain variable region CDR2 of SEQ ID NO: 25; a heavy chain variable region CDR3 of SEQ ID NO: 39; a light chain variable region CDR1 of SEQ ID NO: 53; a light chain variable region CDR2 of SEQ ID NO: 67; and a light chain variable region CDR3 of SEQ ID NO: 81.

In one embodiment, the antibody comprised in the composition employed in the inventive methods for treating urinary incontinence or used in treating urinary incontinence comprises: a heavy chain variable region CDR1 of SEQ ID NO: 12; a heavy chain variable region CDR2 of SEQ ID NO: 26; a heavy chain variable region CDR3 of SEQ ID NO: 40; a light chain variable region CDR1 of SEQ ID NO: 54; a light chain variable region CDR2 of SEQ ID NO: 68; and a light chain variable region CDR3 of SEQ ID NO: 82.

In one embodiment, the antibody comprised in the composition employed in the inventive methods for treating urinary incontinence or used in treating urinary incontinence comprises: a heavy chain variable region CDR1 of SEQ ID NO: 13; a heavy chain variable region CDR2 of SEQ ID NO: 27; a heavy chain variable region CDR3 of SEQ ID NO: 41; a light chain variable region CDR1 of SEQ ID NO: 55; a light chain variable region CDR2 of SEQ ID NO: 69; and a light chain variable region CDR3 of SEQ ID NO: 83.

In one embodiment, the antibody comprised in the composition employed in the inventive methods for treating urinary incontinence or used in treating urinary incontinence comprises: a heavy chain variable region CDR1 of SEQ ID NO: 14; a heavy chain variable region CDR2 of SEQ ID NO: 28; a heavy chain variable region CDR3 of SEQ ID NO: 42; a light chain variable region CDR1 of SEQ ID NO: 56; a light chain variable region CDR2 of SEQ ID NO: 70; and a light chain variable region CDR3 of SEQ ID NO: 84.

In one embodiment, the antibody comprised in the composition employed in the inventive methods for treating urinary incontinence or used in treating urinary incontinence comprises: (a) the variable heavy chain sequence of SEQ ID NO: 85 and variable light chain sequence of SEQ ID NO: 99; (b) the variable heavy chain sequence of SEQ ID NO: 86 and variable light chain sequence of SEQ ID NO: 100; (c) the variable heavy chain sequence of SEQ ID NO: 87 and variable light chain sequence of SEQ ID NO: 101; (d) the variable heavy chain sequence of SEQ ID NO: 88 and variable light chain sequence of SEQ ID NO: 102; (e) the variable heavy chain sequence of SEQ ID NO: 89 and variable light chain sequence of SEQ ID NO: 103; (f) the variable heavy chain sequence of SEQ ID NO: 90 and variable light chain sequence of SEQ ID NO: 104; (g) the variable heavy chain sequence of SEQ ID NO: 91 and variable light chain sequence of SEQ ID NO: 105; (h) the variable heavy chain sequence of SEQ ID NO: 92 and variable light chain sequence of SEQ ID NO: 106; (i) the variable heavy chain sequence of SEQ ID NO: 93 and variable light chain sequence of SEQ ID NO: 107; (j) the variable heavy chain sequence of SEQ ID NO: 94 and variable light chain sequence of SEQ ID NO: 108; (k) the variable heavy chain sequence of SEQ ID NO: 95 and variable light chain sequence of SEQ ID NO: 109; (l) the variable heavy chain sequence of SEQ ID NO: 96 and variable light chain sequence of SEQ ID NO: 110; (m) the variable heavy chain sequence of SEQ ID NO: 97 and variable light chain sequence of SEQ ID NO: 111; or (n) the variable heavy chain sequence of SEQ ID NO: 98 and variable light chain sequence of SEQ ID NO: 112.

In one embodiment, the antibody comprised in the composition employed in the inventive methods for treating urinary incontinence or used in treating urinary incontinence comprises: (a) the heavy chain sequence of SEQ ID NO: 146 and light chain sequence of SEQ ID NO: 141; (b) the heavy chain sequence of SEQ ID NO: 147 and light chain sequence of SEQ ID NO: 142; (c) the heavy chain sequence of SEQ ID NO: 148 and light chain sequence of SEQ ID NO: 143; (d) the heavy chain sequence of SEQ ID NO: 149 and light chain sequence of SEQ ID NO: 144; (e) the heavy chain sequence of SEQ ID NO: 150 and light chain sequence of SEQ ID NO: 145; (f) the heavy chain sequence of SEQ ID NO: 156 and light chain sequence of SEQ ID NO: 151; (g) the heavy chain sequence of SEQ ID NO: 157 and light chain sequence of SEQ ID NO: 152; (h) the heavy chain sequence of SEQ ID NO: 158 and light chain sequence of SEQ ID NO: 153; (i) the heavy chain sequence of SEQ ID NO: 159 and light chain sequence of SEQ ID NO: 154; or (j) the heavy chain sequence of SEQ ID NO: 160 and light chain sequence of SEQ ID NO: 155.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e. greatest % identity) to the sequence of the human antibody. A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g. murine germline sequences). In certain cases, a human antibody may be at least 80%, 90%, or at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

In one embodiment the antibody comprised in the composition employed in the inventive methods for treating urinary incontinence or used in treating urinary incontinence is that antibody encoded by pBW522 or pBW524 (deposited at DSMZ, Inhoffenstr. 7B, D-38124 Braunschweig, Germany on 18 Aug. 2009 under deposit numbers DSM22873 and DSM22874, respectively).

### Homologous Antibodies

In yet another embodiment, the antibody comprised in the composition employed in the inventive methods for treating urinary incontinence or used in treating urinary incontinence has full length heavy and light chain amino acid sequences; full length heavy and light chain nucleotide sequences, variable region heavy and light chain nucleotide sequences, or variable region heavy and light chain amino acid sequences that are homologous to the amino acid and nucleotide sequences of the antibodies described herein, and wherein the antibodies retain the desired functional properties of the anti-ActRIIB antibodies of the disclosure.

For example, the disclosure provides a composition employed in the inventive methods for treating urinary incontinence or used in treating urinary incontinence comprising an isolated recombinant anti-ActRIIB antibody (or a functional protein comprising an antigen binding portion thereof) comprising a heavy chain variable region and a light chain variable region, wherein: the heavy chain variable region comprises an amino acid sequence that is at least 80%, or at least 90% (preferably at least 95, 97 or 99%) identical to an amino acid sequence selected from the group consisting of SEQ ID NOs: 99-112; the light chain variable region comprises an amino acid sequence that is at least 80%, or at least 90% (preferably at least 95, 97 or 99%) identical to an amino acid sequence selected from the group consisting of SEQ ID NOs: 85-98; alternatively the compositions comprises a recombinant anti-ActRIIB antibody (or a functional protein comprising an antigen binding portion thereof) comprising a heavy chain variable region and a light chain variable region, wherein: the heavy chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 99-112; the light chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 85-98 and the antibody exhibits at least one of the following functional properties: (i) it inhibits myostatin binding in vitro or in vivo, (ii) decreases inhibition of muscle differentiation through the Smad-dependent pathway and/or (iii) does not induce hematological changes, in particular no changes in red blood cells absolute count (RBC). In this context, the term “change” refers to insertions, deletions and/or substitutions.

In a further example, the disclosure provides a composition employed in the inventive methods for treating urinary incontinence or used in treating urinary incontinence comprising an isolated recombinant anti-ActRII antibody, (or a functional protein comprising an antigen binding portion thereof) comprising a full length heavy chain and a full length light chain, wherein: the full length heavy chain comprises an amino acid sequence that is at least 80%, or at least 90% (preferably at least 95, 97 or 99%) identical to an amino acid sequence selected from the group consisting of SEQ ID NOs: 146-150 and 156-160; the full length light chain comprises an amino acid sequence that is at least 80%, or at least 90% (preferably at least 95, 97 or 99%) identical to an amino acid sequence selected from the group consisting of SEQ ID NOs: 141-145 and 151-155; alternatively the compositions comprises a recombinant anti-ActRII antibody (or a functional protein comprising an antigen binding portion thereof) comprising a heavy chain variable region and a light chain variable region, wherein: the heavy chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 146-150 and 156-160; the light chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 141-145 and 151-155 and the antibody exhibits at least one of the following functional properties: (i) it inhibits myostatin binding in vitro or in vivo, (ii) decreases inhibition of muscle differentiation through the Smad-dependent pathway and/or (iii) does not induce hematological changes, in particular no changes in RBC. Preferably such an antibody binds to the ligand binding domain of ActRIIB and/or ActRIIA. In this context, the term “change” refers to insertions, deletions and/or substitutions.

In another example, the disclosure provides a composition employed in the inventive methods for treating urinary incontinence or used in treating urinary incontinence comprising an isolated recombinant anti-ActRII antibody (or a functional protein comprising an antigen binding portion thereof), comprising a full length heavy chain and a full length light chain, wherein: the full length heavy chain is encoded by a nucleotide sequence that is at least 80%, or at least 90% (preferably at least 95, 97 or 99%) identical to a nucleotide sequence selected from the group consisting of SEQ ID NOs: 166-170 and 176-180; the full length light chain is encoded by a nucleotide sequence that is at least 80%, or at least 90% (preferably at least 95, 97 or 99%) identical to a nucleotide sequence selected from the group consisting of SEQ ID NOs: 161-165 and 171-175; alternatively the compositions comprises a recombinant anti-ActRIIB antibody (or a functional protein comprising an antigen binding portion thereof) comprising a heavy chain variable region and a light chain variable region, wherein: the heavy chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 166-170 and 176-180; the light chain variable region comprises no more than 5 amino acid, or no more than 4 amino acid, or no more than 3 amino acid, or no more than 2 or no more than 1 amino acid change compared to the amino acid sequence selected from the group consisting of SEQ ID NOs: 161-165 and 171-175 and the antibody exhibits at least one of the following functional properties: (i) it inhibits myostatin binding in vitro or in vivo, (ii) decreases inhibition of muscle differentiation through the Smad-dependent pathway and/or (iii) does not induce hematological changes, in particular no changes in RBC. Preferably such an antibody binds to the ligand binding domain of ActRIIB. In this context, the term “change” refers to insertions, deletions and/or substitutions.

In various embodiments, the antibody comprised in the composition employed in the inventive methods for treating urinary incontinence or used in treating urinary incontinence may exhibit one or more, two or more, or three of the functional properties discussed above. The antibody can be, for example, a human antibody, a humanized antibody or a chimeric antibody. Preferably the antibody is a fully human IgG1 antibody. In other embodiments, the V_{H} and/or V_{L} amino acid sequences may be at least 80%, 90%, 95%, 96%, 97%, 98% or 99% identical to the sequences set forth above. In other embodiments, the V_{H} and/or V_{L} amino acid sequences may be identical except an amino acid substitution in no more than 1, 2, 3, 4 or 5 amino acid position. An antibody having V_{H} and V_{L} regions having high (i.e. 80% or greater) identity to the V_{H} and V_{L} regions of SEQ ID NOs 99-112 and SEQ ID NOs: 85-98 respectively, can be obtained by mutagenesis (e.g. site-directed or PCR-mediated mutagenesis) of nucleic acid molecules SEQ ID NOs: 127-140 and 113-126 respectively, followed by testing of the encoded altered antibody for retained function (i.e. the functions set forth above) using the functional assays described herein.

In other embodiments, the full length heavy chain and/or full length light chain amino acid sequences of an antibody employed in the inventive methods for treating urinary incontinence or used in treating urinary incontinence may be at least 80%, 90%, 95%, 96%, 97%, 98% or 99% identical to the sequences set forth above or may be identical except an amino acid change in no more than 1, 2, 3, 4 or 5 amino acid position. An antibody having a full length heavy chain and full length light chain having high (i.e. at least 80% or greater) identity to the full length heavy chains of any of SEQ ID NOs: 146-150 and 156-160 and full length light chains of any of SEQ ID NOs: 141-145 and 151-155 respectively, can be obtained by mutagenesis (e.g. site-directed or PCR-mediated mutagenesis) of nucleic acid molecules SEQ ID NOs: 166-170 and 176-180 and SEQ ID NOs: 161-165 and 171-175 respectively, followed by testing of the encoded altered antibody for retained function (i.e. the functions set forth above) using the functional assays described herein.

In other embodiments, the full length heavy chain and/or full length light chain nucleotide sequences of an antibody employed in the inventive methods for treating urinary incontinence or used in treating urinary incontinence may be at least 80%, 90%, 95%, 96%, 97%, 98% or 99% identical to the sequences set forth above.

In other embodiments, the variable regions of heavy chain and/or light chain nucleotide sequences of an antibody employed in the inventive methods for treating urinary incontinence or used in treating urinary incontinence may be at least 80%, 90%, 95%, 96%, 97%, 98% or 99% identical to the sequences set forth above or may be identical except an amino acid change in no more than 1, 2, 3, 4 or 5 amino acid position.

As used herein, the percent identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Antibodies with Conservative Modifications

In certain embodiments, an antibody comprised in the composition employed in the inventive methods for treating urinary incontinence or used in treating urinary incontinence has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or variant sequences thereof comprising 1, 2, 3, 4 or 5 amino acid changes or conservative modifications thereof, and wherein the antibodies retain the desired functional properties of the anti-ActRIIB antibodies of the disclosure. Accordingly, the disclosure provides compositions employed in the inventive methods for treating urinary incontinence or used in treating urinary incontinence comprising an isolated recombinant anti-ActRIIB antibody, or a functional protein comprising an antigen binding portion thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 1-14 or variant sequences thereof comprising 1, 2, 3, 4 or 5 amino acid changes, and conservative modifications thereof; the heavy chain variable region CDR2 amino acid sequences are selected from the group consisting of SEQ ID NOs: 15-28 or variant sequences thereof comprising 1, 2, 3, 4 or 5 amino acid changes, and conservative modifications thereof; the heavy chain variable region CDR3 amino acid sequences are selected from the group consisting of SEQ ID NOs: 29-42 or variant sequences thereof comprising 1, 2, 3, 4 or 5 amino acid changes, and conservative modifications thereof; the light chain variable regions CDR1 amino acid sequences are selected from the group consisting of SEQ ID NOs: 43-56 or variant sequences thereof comprising 1, 2, 3, 4 or 5 amino acid changes, and conservative modifications thereof; the light chain variable regions CDR2 amino acid sequences are selected from the group consisting of SEQ ID NOs: 57-70 or variant sequences thereof comprising 1, 2, 3, 4 or 5 amino acid changes, and conservative modifications thereof; the light chain variable regions of CDR3 amino acid sequences are selected from the group consisting of SEQ ID NOs: 71-84 or variant sequences thereof comprising 1, 2, 3, 4 or 5 amino acid changes, and conservative modifications thereof. Preferably the antibody exhibits at least one of the following functional properties: (i) it inhibits myostatin binding in vitro or in vivo, (ii) decreases inhibition of muscle differentiation through the Smad-dependent pathway and/or (iii) does not induce hematological changes, in particular, no changes in RBC.

In various embodiments, the antibody employed in the inventive methods for treating urinary incontinence or used in treating urinary incontinence may exhibit one or both of the functional properties listed above. Such antibodies can be, for example, human antibodies, humanized antibodies or chimeric antibodies.

In other embodiments, an antibody comprised in the composition employed in the inventive methods for treating urinary incontinence or used in treating urinary incontinence is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the anti-ActRIIB antibodies of the disclosure. Accordingly, the disclosure provides compositions employed in the inventive methods for treating urinary incontinence or used in treating urinary incontinence comprising an isolated monoclonal anti-ActRII antibody optimized for expression in a mammalian cell consisting of a full length heavy chain and a full length light chain wherein: the full length heavy chain has amino acid sequences selected from the group of SEQ ID NOs: 146-150 and 156-160 or variant sequences thereof comprising 1, 2, 3, 4 or 5 amino acid changes, and conservative modifications thereof; and the full length light chain has amino acid sequences selected from the group of SEQ ID NOs: 141-145 and 151-155 or variant sequences thereof comprising 1, 2, 3, 4 or 5 amino acid changes, and conservative modifications thereof; and the antibody exhibits at least one of the following functional properties: (i) it inhibits myostatin binding in vitro or in vivo, (ii) decreases inhibition of muscle differentiation through the Smad-dependent pathway and/or (iii) does not induce hematological changes, in particular no changes in RBC.

In various embodiments, the antibody may exhibit one or both of the functional properties listed above. Such antibodies can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, one or more amino acid residues within the CDR regions of an antibody of the disclosure can be replaced with other amino acid residues from the same side chain family, and the altered antibody can be tested for retained function using the functional assays described herein.

Antibodies that Bind to the Same Epitope as Anti-ActRII Antibodies Comprised in the Disclosed Composition

In another embodiment, the disclosure provides the use of compositions in the inventive methods for treating urinary incontinence or used in treating urinary incontinence comprising antibodies that bind to the same epitope as the various specific anti-ActRII antibodies described herein. All the antibodies described in the examples that are capable of blocking myostatin binding to ActRIIA and ActRIIB bind to one of the epitopes in ActRIIA and ActRIIB with high affinity, said epitope being comprised between amino acids 19-134 of SEQ ID NO:181.

Additional antibodies can therefore be identified based on their ability to cross-compete (e.g. to competitively inhibit the binding of, in a statistically significant manner) with other antibodies of the disclosure in standard ActRIIB binding assays. The ability of a test antibody to inhibit the binding of antibodies comprised in the inventive compositions to human ActRIIB demonstrates that the test antibody can compete with said antibody for binding to human ActRIIB; such an antibody may, according to non-limiting theory, bind to the same or a related (e.g. a structurally similar or spatially proximal) epitope on human ActRIIB as the antibody with which it competes. In a certain embodiment, the antibody that binds to the same epitope on human ActRIIB and ActRIIA as the antibodies comprised in the compositions employed in the inventive methods for treating urinary incontinence or used in treating urinary incontinence is a human recombinant antibody. Such human recombinant antibodies can be prepared and isolated as described in the examples.

Thus, the disclosure provides a composition for use in the inventive methods for treating urinary incontinence or used in treating urinary incontinence comprising an antibody that binds to an epitope recognized by and/or that competes for binding with an antibody having the variable heavy chain sequence recited in SEQ ID NO: 85, and the variable light chain sequence recited in SEQ ID NO: 99.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 86, and the variable light chain sequence recited in SEQ ID NO: 100.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 87, and the variable light chain sequence recited in SEQ ID NO: 101.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 88, and the variable light chain sequence recited in SEQ ID NO: 102.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 89, and the variable light chain sequence recited in SEQ ID NO: 103.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 90, and the variable light chain sequence recited in SEQ ID NO: 104.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 91, and the variable light chain sequence recited in SEQ ID NO: 105.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 92, and the variable light chain sequence recited in SEQ ID NO: 106.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 93, and the variable light chain sequence recited in SEQ ID NO: 107.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 94, and the variable light chain sequence recited in SEQ ID NO: 108.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 95, and the variable light chain sequence recited in SEQ ID NO: 109.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 96, and the variable light chain sequence recited in SEQ ID NO: 110.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 97, and the variable light chain sequence recited in SEQ ID NO: 111.

Thus, the disclosure provides a composition for use in the inventive methods for treating urinary incontinence or used in treating urinary incontinence comprising an antibody that binds to an epitope recognized by an antibody having the variable heavy chain sequence recited in SEQ ID NO: 98, and the variable light chain sequence recited in SEQ ID NO: 112.

Following more detailed epitope mapping experiments, the binding regions of preferred antibodies of the inventive compositions have been more clearly defined.

Thus, the disclosure provides a composition for use in the inventive methods for treating urinary incontinence or used in treating urinary incontinence comprising an antibody that binds to an epitope comprising amino acids 78-83 of SEQ ID NO: 181 (WLDDFN—SEQ ID NO:188).

The disclosure also provides a composition for use in the inventive methods for treating urinary incontinence or used in treating urinary incontinence comprising an antibody that binds to an epitope comprising amino acids 76-84 of SEQ ID NO: 181 (GCWLDDFNC—SEQ ID NO:186).

The disclosure also provides a composition for use in the inventive methods for treating urinary incontinence or used in treating urinary incontinence comprising an antibody that binds to an epitope comprising amino acids 75-85 of SEQ ID NO: 181 (KGCWLDDFNCY—SEQ ID NO:190).

The disclosure also provides a composition for use in the inventive methods for treating urinary incontinence or used in treating urinary incontinence comprising an antibody that binds to an epitope comprising amino acids 52-56 of SEQ ID NO: 181 (EQDKR—SEQ ID NO:189).

The disclosure also provides a composition for use in the inventive methods for treating urinary incontinence or used in treating urinary incontinence comprising an antibody that binds to an epitope comprising amino acids 49-63 of SEQ ID NO: 181 (CEGEQDKRLHCYASW—SEQ ID NO:187).

The disclosure also provides a composition for use in the inventive methods for treating urinary incontinence or used in treating urinary incontinence comprising an antibody that binds to an epitope comprising or consisting of amino acids 29-41 of SEQ ID NO: 181 (CIYYNANWELERT—SEQ ID NO:191).

The disclosure also provides a composition for use in the inventive methods for treating urinary incontinence or used in treating urinary incontinence comprising an antibody that binds to an epitope comprising or consisting of amino acids 100-110 of SEQ ID NO: 181 (YFCCCEGNFCN—SEQ ID NO:192).

The disclosure also provides a composition for use in the inventive methods for treating urinary incontinence or used in treating urinary incontinence comprising antibodies that bind to epitopes consisting of these sequences or epitopes comprising combinations of these epitope regions.

Thus, the disclosure also provides a composition for use in the inventive methods for treating urinary incontinence or used in treating urinary incontinence comprising an antibody that binds to an epitope comprising or consisting of amino acids 78-83 of SEQ ID NO: 181 (WLDDFN) and amino acids 52-56 of SEQ ID NO: 181 (EQDKR).

### Engineered and Modified Antibodies

An antibody comprised in the compositions for use in the inventive methods for treating urinary incontinence or used in treating urinary incontinence further can be prepared using an antibody having one or more of the V_{H} and/or V_{L} sequences shown herein as starting material to engineer a modified antibody, which modified antibody may have altered properties from the starting antibody. An antibody can be engineered by modifying one or more residues within one or both variable regions (i.e. V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g. Riechmann, L. et al., 1998 Nature 332:323-327; Jones, P. et al., 1986 Nature 321:522-525; Queen, C. et al., 1989 Proc. Natl. Acad. Sci. U.S.A. 86:10029-10033; U.S. Pat. No. 5,225,539 to Winter, and U.S. Pat. Nos. 5,530,101; 5,585,089; 5,693,762 and 6,180,370 to Queen et al.).

Accordingly, another embodiment of the disclosure pertains to the use of compositions in the inventive methods for treating urinary incontinence or used in treating urinary incontinence comprising a monoclonal anti-ActRII antibody, or a functional protein comprising an antigen binding portion thereof, comprising a heavy chain variable region comprising CDR1 sequences having an amino acid sequence selected from the group consisting of SEQ ID NOs: 1-14; CDR2 sequences having an amino acid sequence selected from the group consisting of SEQ ID NOs: 15-28; CDR3 sequences having an amino acid sequence selected from the group consisting of SEQ ID NOs: 29-42, respectively; and a light chain variable region having CDR1 sequences having an amino acid sequence selected from the group consisting of SEQ ID NOs: 43-56; CDR2 sequences having an amino acid sequence selected from the group consisting of SEQ ID NOs: 57-70; and CDR3 sequences consisting of an amino acid sequence selected from the group consisting of SEQ ID NOs: 71-84, respectively. Thus, such antibodies contain the V_{H} and V_{L} CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al., [supra]; Tomlinson, I. M., et al., 1992 J. fol. Biol. 227:776-798; and Cox, J. P. L. et al., 1994 Eur. J Immunol. 24:827-836.

An example of framework sequences for use in the antibodies of the disclosure are those that are structurally similar to the framework sequences used by selected antibodies of the disclosure, e.g. consensus sequences and/or framework sequences used by monoclonal antibodies of the disclosure. The V_{H} CDR1, 2 and 3 sequences, and the V_{L} CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g. U.S. Pat. Nos. 5,530,101; 5,585,089; 5,693,762 and 6,180,370 to Queen et a).

Another type of variable region modification is to mutate amino acid residues within the V_{H} and/or V_{L} CDR1, CDR2 and/or CDR3 regions to thereby improve one or more binding properties (e.g. affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Accordingly, in another embodiment, the disclosure provides the use of isolated human anti-ActRII monoclonal antibodies, or a functional protein comprising an antigen binding portion thereof, in the inventive methods for treating urinary incontinence or used in treating urinary incontinence, consisting of a heavy chain variable region having: a V_{H} CDR1 region consisting of an amino acid sequence selected from the group having SEQ ID NOs: 1-14 or an amino acid sequence having one, two, three, four or five amino acid substitutions, deletions or additions as compared to SEQ ID NOs: 1-14; a V_{H} CDR2 region having an amino acid sequence selected from the group consisting of SEQ ID NOs: 15-28, or an amino acid sequence having one, two, three, four or five amino acid substitutions, deletions or additions as compared to SEQ ID NOs: 15-28; a V_{H} CDR3 region having an amino acid sequence selected from the group consisting of SEQ ID NOs: 29-42, or an amino acid sequence having one, two, three, four or five amino acid substitutions, deletions or additions as compared to SEQ ID NOs: 29-42; a V_{L} CDR1 region having an amino acid sequence selected from the group consisting of SEQ ID NOs: 43-56, or an amino acid sequence having one, two, three, four or five amino acid substitutions, deletions or additions as compared to SEQ ID NOs: 43-56; a V_{L} CDR2 region having an amino acid sequence selected from the group consisting of SEQ ID NOs: 52-70, or an amino acid sequence having one, two, three, four or five amino acid substitutions, deletions or additions as compared to SEQ ID NOs: 52-70; and a V_{L} CDR3 region having an amino acid sequence selected from the group consisting of SEQ ID NOs: 71-84, or an amino acid sequence having one, two, three, four or five amino acid substitutions, deletions or additions as compared to SEQ ID NOs: 71-84.

### Camelid Antibodies

Antibody proteins obtained from members of the camel and dromedary family (Camelus bactrianus and Camelus dromaderius) including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals (see WO94/04678).

A region of the camelid antibody which is the small single variable domain identified as V_{HH} can be obtained by genetic engineering to yield a small protein having high affinity for a target, resulting in a low molecular weight antibody-derived protein known as a “camelid nanobody” (see U.S. Pat. No. 5,759,808; Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520). Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e. the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e. camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier (see US2004/0161738). These features combined with the low antigenicity to humans indicate great therapeutic potential. Further, these molecules can be fully expressed in prokaryotic cells such as E. coli and are expressed as fusion proteins with bacteriophage and are functional.

Accordingly, in one embodiment, the present disclosure relates to the use of compositions comprising a camelid antibody or nanobody having high affinity for ActRIIB in the inventive methods for treating urinary incontinence or used in treating urinary incontinence. In certain embodiments herein, the camelid antibody or nanobody is naturally produced in the camelid animal, i.e. is produced by the camelid following immunization with ActRIIB or a peptide fragment thereof, using techniques described herein for other antibodies. Alternatively, the anti-ActRIIB camelid nanobody is engineered, i.e. produced by selection for example from a library of phage displaying appropriately mutagenized camelid nanobody proteins using panning procedures with ActRIIB as a target as described in the examples herein. Engineered nanobodies can further be customized by genetic engineering to have a half-life in a recipient subject of from 45 minutes to two weeks. In a specific embodiment, the camelid antibody or nanobody used in the inventive methods for treating urinary incontinence or used in treating urinary incontinence is obtained by grafting the CDRs sequences of the heavy or light chain of the human antibodies of the disclosure into nanobody or single domain antibody framework sequences, as described for example in WO94/04678.

### Non-Antibody Scaffold

Known non-immunoglobulin frameworks or scaffolds include, but are not limited to, Adnectins (fibronectin) (Compound Therapeutics, Inc., Waltham, Mass.), ankyrin (Molecular Partners AG, Zurich, Switzerland), domain antibodies (Domantis, Ltd (Cambridge, Mass.) and Ablynx nv (Zwijnaarde, Belgium)), lipocalin (Anticalin) (Pieris Proteolab AG, Freising, Germany), small modular immuno-pharmaceuticals (Trubion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Scil Proteins GmbH, Halle, Germany), protein epitope mimetics (Polyphor Ltd, Allschwil, Switzerland).

### (i) Fibronectin Scaffold

The fibronectin scaffolds are based preferably on fibronectin type III domain (e.g. the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (U.S. Pat. No. 6,818,418).

These fibronectin-based scaffolds are not an immunoglobulin, although the overall fold is closely related to that of the smallest functional antibody fragment, the variable region of the heavy chain, which comprises the entire antigen recognition unit in camel and llama IgG. Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the disclosure using standard cloning techniques.

### (ii) Ankyrin—Molecular Partners

The technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

(iii) Maxybodies/Avimers—Avidia

Avimers are derived from natural A-domain containing protein such as LRP-1. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, US2004/0175756; US2005/0053973; US2005/0048512; and US2006/0008844.

### (vi) Protein A—Affibody

Affibody® affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate Affibody® libraries with a large number of ligand variants (See e.g. U.S. Pat. No. 5,831,012). Affibody® molecules mimic antibodies, they have a molecular weight of 6 kDa, compared to the molecular weight of antibodies, which is 150 kDa. In spite of its small size, the binding site of Affibody® molecules is similar to that of an antibody.

### (v) Anticalins—Pieris

Anticalins® are products developed by the company Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids.

The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain.

The set of four loops, which makes up the binding pocket, shows pronounced structural plasticity and tolerates a variety of side chains. The binding site can thus be reshaped in a proprietary process in order to recognize prescribed target molecules of different shape with high affinity and specificity.

One protein of lipocalin family, the bilin-binding protein (BBP) of Pieris brassicae has been used to develop anticalins by mutagenizing the set of four loops. One example of a patent application describing “anticalins” is WO1999/16873.

### (vi) Affilin—Scil Proteins

AFFILIN™ molecules are small non-immunoglobulin proteins which are designed for specific affinities towards proteins and small molecules. New AFFILIN™ molecules can be very quickly selected from two libraries, each of which is based on a different human derived scaffold protein.

AFFILIN™ molecules do not show any structural homology to immunoglobulin proteins. Scil Proteins employs two AFFILIN™ scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO2001/004144 and examples of “ubiquitin-like” proteins are described in WO2004/106368.

(vii) Protein Epitope Mimetics (PEM)

PEM are medium-sized, cyclic, peptide-like molecules (MW 1-2 kDa) mimicking beta-hairpin secondary structures of proteins, the major secondary structure involved in protein-protein interactions.

Grafting Antigen-Binding Domains into Alternative Frameworks or Scaffolds

A wide variety of antibody/immunoglobulin frameworks or scaffolds can be employed so long as the resulting polypeptide includes at least one binding region which specifically binds to ActRIIB. Such frameworks or scaffolds include the 5 main idiotypes of human immunoglobulins, or fragments thereof (such as those disclosed elsewhere herein), and include immunoglobulins of other animal species, preferably having humanized aspects. Single heavy-chain antibodies such as those identified in camelids are of particular interest in this regard. Novel frameworks, scaffolds and fragments continue to be discovered and developed by those skilled in the art.

In one aspect, the compositions for use in the inventive methods for treating urinary incontinence or used in treating urinary incontinence may comprise non-immunoglobulin based antibodies using non-immunoglobulin scaffolds onto which CDRs of the disclosed antibodies can be grafted. Known or future non-immunoglobulin frameworks and scaffolds may be employed, as long as they comprise a binding region specific for the target protein of SEQ ID NO: 181 (preferably, the ligand binding domain thereof as shown in SEQ ID NO: 182). Such compounds are known herein as “polypeptides comprising a target-specific binding region”. Examples of non-immunoglobulin framework are further described in the sections below (camelid antibodies and non-antibody scaffold).

### Framework or Fc Engineering

Engineered antibodies comprised in the compositions for use in the inventive methods for treating urinary incontinence or used in treating urinary incontinence include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies can also be comprised in the compositions of the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in US2003/0153043.

In addition or alternative to modifications made within the framework or CDR regions, antibodies for use in the inventive methods for treating urinary incontinence or used in treating urinary incontinence may be engineered to include modifications within the Fc region, typically to alter one or more functional properties of the antibody, such as serum half-life, complement fixation, Fc receptor binding, and/or antigen-dependent cellular cytotoxicity. Furthermore, an antibody comprised in the compositions of the disclosure may be chemically modified (e.g. one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g. increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In another embodiment, the antibody used in the inventive methods for treating urinary incontinence or used in treating urinary incontinence is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al. In particular, residues 234 and 235 may be mutated. In particular, these mutations may be to alanine. Thus in one embodiment the antibody comprised in the compositions for use in the inventive methods for treating urinary incontinence or used in treating urinary incontinence has a mutation in the Fc region at one or both of amino acids 234 and 235. In another embodiment, one or both of amino acids 234 and 235 may be substituted to alanine. Substitution of both amino acids 234 and 235 to alanine results in a reduced ADCC activity.

In another embodiment, one or more amino acids selected from amino acid residues of the described antibodies can be replaced with a different amino acid residue such that the antibody has altered C1q binding and/or reduced or abolished complement dependent cytotoxicity (CDC). This approach is described in further detail in U.S. Pat. No. 6,194,551.

In another embodiment, one or more amino acid residues of the described antibodies are altered to thereby alter the ability of the antibody to fix complement. This approach is described further in WO94/29351.

In yet another embodiment, the Fc region of the described antibodies is modified to increase the ability of the antibody to mediate antibody dependent cellular cytotoxicity (ADCC) and/or to increase the affinity of the antibody for an Fcγ receptor by modifying one or more amino acids. This approach is described further in WO00/42072. Moreover, the binding sites on human IgG1 for FcγRI, FcγRII, FcγRII and FcRn have been mapped and variants with improved binding have been described (see Shields, R. L. et al., 2001 J. Biol. Chen. 276:6591-6604).

In still another embodiment, the glycosylation of an antibody comprised in the compositions of the disclosure is modified. For example, an aglycoslated antibody can be made (i.e. the antibody lacks glycosylation). Glycosylation can be altered to, for example, increase the affinity of the antibody for the antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Another modification of the antibodies for use in the inventive methods for treating urinary incontinence or used in treating urinary incontinence that is contemplated by the disclosure is a conjugate or a protein fusion of at least the antigen-binding region of said antibodies to a serum protein, such as human serum albumin or a fragment thereof to increase half-life of the resulting molecule (see, for example, EP0322094).

Another possibility is a fusion of at least the antigen-binding region of the antibody comprised in the composition of the disclosure to proteins capable of binding to serum proteins, such as human serum albumin to increase half-life of the resulting molecule (see, for example, EP0486525).

### Methods of Engineering Altered Antibodies

As discussed above, the anti-ActRIIB antibodies having CDR sequences, V_{H} and V_{L} sequences or full length heavy and light chain sequences shown herein can be used to create new anti-ActRIIB antibodies by modifying the CDR sequences full length heavy chain and/or light chain sequences, V_{H} and/or V_{L} sequences, or the constant region(s) attached thereto. Thus, in another aspect of the disclosure, the structural features of an anti-ActRIIB antibody comprised in the compositions for use in the inventive methods for treating urinary incontinence or used in treating urinary incontinence are used to create structurally related anti-ActRIIB antibodies that retain at least one functional property of said antibodies used in the inventive methods, such as binding to human ActRIIB but also inhibit one or more functional properties of ActRIIB (for example, the inhibition of Smad activation).

For example, one or more CDR regions of the antibodies comprised in the compositions for use in the inventive methods for treating urinary incontinence or used in treating urinary incontinence of the present disclosure, or mutations thereof, can be combined recombinantly with known framework regions and/or other CDRs to create additional, recombinantly-engineered, anti-ActRIIB antibodies comprised in the compositions of the disclosure, as discussed above. Other types of modifications include those described in the previous section. The starting material for the engineering method is one or more of the V_{H} and/or V_{L} sequences provided herein, or one or more CDR regions thereof. To create the engineered antibody, it is not necessary to actually prepare (i.e. express as a protein) an antibody having one or more of the V_{H} and/or V_{L} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

The altered antibody sequence can also be prepared by screening antibody libraries having fixed CDR3 sequences selected among the group consisting of SEQ ID NO: 29-42 and SEQ ID NO: 71-84 or minimal essential binding determinants as described in US2005/0255552 and diversity on CDR1 and CDR2 sequences. The screening can be performed according to any screening technology appropriate for screening antibodies from antibody libraries, such as phage display technology.

Standard molecular biology techniques can be used to prepare and express the altered antibody sequence. The antibody encoded by the altered antibody sequence(s) is one that retains one, some or all of the functional properties of the anti-ActRIIB antibodies described herein, which functional properties include, but are not limited to, specifically binding to human ActRIIB and inhibition of Smad activation.

The altered antibody may exhibit one or more, two or more, or three or more of the functional properties discussed above.

The functional properties of the altered antibodies can be assessed using standard assays available in the art and/or described herein, such as those set forth in the Examples (e.g. ELISAs).

Mutations can be introduced randomly or selectively along all or part of an anti-ActRIIB antibody coding sequence and the resulting modified anti-ActRIIB antibodies can be screened for binding activity and/or other functional properties as described herein. Mutational methods have been described in the art. For example, WO02/092780 describes methods for creating and screening antibody mutations using saturation mutagenesis, synthetic ligation assembly, or a combination thereof. Alternatively, WO03/074679 describes methods of using computational screening methods to optimize physiochemical properties of antibodies.

### Nucleic Acid Molecules Encoding Antibodies Comprised in the Compositions of the Disclosure

Examples of full length light chain nucleotide sequences optimized for expression in a mammalian cell are shown in SEQ ID NOs: 161-165 and 171-175. Examples of full length heavy chain nucleotide sequences optimized for expression in a mammalian cell are shown in SEQ ID NOs: 166-170 and 176-180.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g. other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. Nucleic acids can be obtained using standard molecular biology techniques. For antibodies expressed by hybridomas (e.g. hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g. using phage display techniques), nucleic acids encoding the antibodies can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g. Kabat, E. A., et al. [supra]) and DNA fragments encompassing these regions can be obtained by standard PCR amplification. The heavy chain constant region can be an IgG1, IgG2, IgG3, IgG4, IgA, IgE, IgM or IgD constant region. The heavy chain constant region can be selected among IgG1 isotypes. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g. Kabat, E. A., et al. [supra]) and DNA fragments encompassing these regions can be obtained by standard PCR amplification. The light chain constant region can be a kappa or a lambda constant region.

To create an scFv gene, the V_{H}- and V_{L}-encoding DNA fragments are operatively linked to another fragment encoding a flexible linker, e.g. encoding the amino acid sequence (Gly4-Ser)₃, such that the V_{H} and V_{L} sequences can be expressed as a contiguous single-chain protein, with the V_{L} and V_{H} regions joined by the flexible linker (see e.g. Bird et al., 1988 Science 242:423-426; Huston et al., 1988 Proc. Natl. Acad. Sci. USA 85:5879-5883; McCafferty et al., 1990 Nature 348:552-554).

### Generation of Monoclonal Antibodies

Monoclonal antibodies (mAbs) can be produced by a variety of techniques, including conventional monoclonal antibody methodology e.g. the standard somatic cell hybridization technique of Kohler and Milstein (1975 Nature 256: 495). Many techniques for producing monoclonal antibody can be employed e.g. viral or oncogenic transformation of B lymphocytes.

An animal system for preparing hybridomas is the murine system. Hybridoma production in the mouse is a well-established procedure. Immunization protocols and techniques for isolation of immunized splenocytes for fusion are known in the art. Fusion partners (e.g. murine myeloma cells) and fusion procedures are also known.

Chimeric or humanized antibodies comprised in the compositions for use in the inventive methods for treating urinary incontinence or used in treating urinary incontinence can be prepared based on the sequence of a murine monoclonal antibody prepared as described above. DNA encoding the heavy and light chain immunoglobulins can be obtained from the murine hybridoma of interest and engineered to contain non-murine (e.g. human) immunoglobulin sequences using standard molecular biology techniques. For example, to create a chimeric antibody, the murine variable regions can be linked to human constant regions using methods known in the art (see e.g. U.S. Pat. No. 4,816,567). To create a humanized antibody, the murine CDR regions can be inserted into a human framework using methods known in the art (see e.g. U.S. Pat. Nos. 5,225,539; 5,530,101; 5,585,089; 5,693,762 and 6,180,370).

In a certain embodiment, the antibodies comprised in the compositions for use in the inventive methods for treating urinary incontinence or used in treating urinary incontinence are human monoclonal antibodies. Such human monoclonal antibodies directed against ActRIIB can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

(see e.g. Lonberg, et al., 1994 Nature 368(6474): 856-859). See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as WO92/103918, WO93/12227, WO94/25585, WO97/113852, WO98/24884; WO99/45962; and WO01/14424.

In another embodiment, human antibodies comprised in the compositions for use in the inventive methods for treating urinary incontinence or used in treating urinary incontinence can be raised using a mouse that carries human immunoglobulin sequences on transgenes and transchromosomes such as a mouse that carries a human heavy chain transgene and a human light chain transchromosome. Such mice, referred to herein as “KM mice”, are described in detail in WO02/43478.

Still further, alternative transgenic animal systems expressing human immunoglobulin genes are available in the art and can be used to raise anti-ActRIIB antibodies of the disclosure. For example, an alternative transgenic system referred to as the Xenomouse (Abgenix, Inc.) can be used. Such mice are described in, e.g. U.S. Pat. Nos. 5,939,598; 6,075,181; 6,114,598; 6, 150,584 and 6,162,963.

Human recombinant antibodies comprised in the compositions of the disclosure can also be prepared using phage display methods for screening libraries of human immunoglobulin genes. Such phage display methods for isolating human antibodies are established in the art or described in the examples below. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915 and 6,593,081.

Human monoclonal antibodies comprised in the compositions for use in the inventive methods for treating urinary incontinence or used in treating urinary incontinence can also be prepared using SCID mice into which human immune cells have been reconstituted such that a human antibody response can be generated upon immunization. Such mice are described in, for example, U.S. Pat. Nos. 5,476,996 and 5,698,767.

### Generation of Hybridomas Producing Human Monoclonal Antibodies

To generate hybridomas producing human monoclonal antibodies comprised in the compositions for use in the inventive methods for treating urinary incontinence or used in treating urinary incontinence, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two-week incubation in selective medium containing 20% fetal Clone Serum, 18% “653” conditioned media, 5% origen (IGEN), 4 mM L-glutamine, 1 mM sodium pyruvate, 5 mM HEPES, 0:055 mM 2-mercaptoethanol, 50 units/ml penicillin, 50 mg/ml streptomycin, 50 mg/ml gentamycin and 1× HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

### Generation of Transfectomas Producing Monoclonal Antibodies

Antibodies comprised in the compositions for use in the inventive methods for treating urinary incontinence or used in treating urinary incontinence also can be produced in a host cell transfectoma using, for example, a combination of recombinant DNA techniques and gene transfection methods as is well known in the art (e.g. Morrison, S. (1985) Science 229:1202).

For example, to express the antibodies, or antibody fragments thereof, DNAs encoding partial or full-length light and heavy chains, can be obtained by standard molecular biology techniques (e.g. PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g. ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e. a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers and other expression control elements (e.g. polyadenylation signals) that control the transcription or translation of the antibody chain genes. Such regulatory sequences are described, for example, in Goeddel (Gene Expression Technology. Methods in Enzymology 185, Academic Press, San Diego, Calif. 1990). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g. the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al., 1988 Mol. Cell. Biol. 8:466-472).

In addition to the antibody chain genes and regulatory sequences, the recombinant expression vectors may carry additional sequences, such as sequences that regulate replication of the vector in host cells (e.g. origins of replication) and selectable marker genes. The selectable marker gene facilitates selection of host cells into which the vector has been introduced (see, e.g. U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g. electroporation, calcium-phosphate precipitation, DEAE-dextran transfection and the like. It is theoretically possible to express the antibodies of the disclosure in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R., 1985 Immunology Today 6:12-13).

Mammalian host cells for expressing the recombinant antibodies comprised in the compositions of the disclosure include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g. as described in R. J. Kaufman and P. A. Sharp, 1982 Mol. Biol. 159:601-621), NSO myeloma cells, COS cells and SP2 cells. In one embodiment, the host cells are CHO K1PD cells. In particular, for use with NSO myeloma cells, another expression system is the GS gene expression system shown in WO87/04462, WO89/01036 and EP 338,841. Mammalian host cells for expressing the recombinant antibodies comprised in the compositions of the disclosure include mammalian cell lines deficient for FUT8 gene expression, for example as described in U.S. Pat. No. 6,946,292B2. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

### Pharmaceutical Compositions

In another aspect, the present disclosure provides a composition, e.g. a pharmaceutical composition, for use in the inventive methods for treating urinary incontinence or used in treating urinary incontinence containing one or a combination of the above described antibodies/monoclonal antibodies, or antigen-binding portion(s) thereof, formulated together with a pharmaceutically acceptable carrier. Such compositions may include one or a combination of (e.g. two or more different) the described antibodies, or immunoconjugates or bispecific molecules. For example, a pharmaceutical composition for use in the inventive methods for treating urinary incontinence or used in treating urinary incontinence can comprise a combination of antibodies that bind to different epitopes on the target antigen or that have complementary activities.

Pharmaceutical compositions for use in the inventive methods for treating urinary incontinence or used in treating urinary incontinence also can be administered in combination therapy, ie. combined with other agents. For example, the combination therapy can include an anti-ActRII antibody of the present disclosure combined with at least one other muscle mass/strength increasing agent, for example, IGF-1 or variants of IGF-1, an anti-myostatin antibody, a myostatin propeptide, a myostatin decoy protein that binds ActRIIB but does not activate it, a beta 2 agonist, a Ghrelin agonist, a SARM, GH agonists/mimetics or follistatin. Examples of therapeutic agents that can be used in combination therapy are described in greater detail below in the section on uses of the antibodies of the disclosure.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g. by injection or infusion), preferably for intravenous injection or infusion. Depending on the route of administration, the active compound, i.e. antibody, immunoconjugate, or bispecific molecule, may be coated in a material to protect the compound from the action of acids and other natural conditions that may inactivate the compound.

A pharmaceutical composition for use in the inventive methods for treating urinary incontinence or used in treating urinary incontinence also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions for use in the inventive methods for treating urinary incontinence or used in treating urinary incontinence may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of agents enumerated above, as required, followed by sterilization microfiltration. Generally, dispersions are prepared by incorporating the active compound into a sterile vehicle that contains a basic dispersion medium and the required other agents from those enumerated above. In the case of sterile powders for the preparation of sterile injectable solutions, the methods of preparation are vacuum drying and freeze-drying (lyophilization) that yield a powder of the active agent plus any additional desired agent from a previously sterile-filtered solution thereof.

The amount of active agent which can be combined with a carrier material to produce a single dosage form will vary depending upon the subject being treated, and the particular mode of administration. The amount of active agent which can be combined with a carrier material to produce a single dosage form will generally be that amount of the composition which produces a therapeutic effect. Generally, out of one hundred percent, this amount will range from about 0.01 percent to about ninety-nine percent of active agent, from about 0.1 percent to about 70 percent, or from about 1 percent to about 30 percent of active agent in combination with a pharmaceutically acceptable carrier.

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of sensitivity in individuals.

For administration of the antibody comprising composition for use in the inventive methods for treating urinary incontinence or used in treating urinary incontinence, the antibody dosage ranges from about 0.0001 to about 100 mg/kg, and more usually about 0.01 to about 30 mg/kg, of the host body weight. For example, dosages are about 1 mg/kg body weight, about 3 mg/kg body weight, about 5 mg/kg body weight or about 10 mg/kg body weight within the ranges of about 1-10 mg/kg e.g., about 1, 2, 3, 4, 5, 6, 7, 8, 9, 10 mg/kg body weight. Dosages are repeated as necessary and may be in the range from about once per week up to about once every 10 weeks, e.g., once every 4 to 8 weeks.

Administration is for example carried out intravenously. Dosage regimens for an anti-ActRII antibody for use in the inventive methods for treating urinary incontinence or used in treating urinary incontinence, e.g., bimagrumab, include about 1 mg/kg body weight or about 3 mg/kg body weight or about 10 mg/kg body weight, once every four weeks by intravenous administration.

Administration is for example carried out subcutaneously. Dosage regimens for an anti-ActRII antibody for use in the inventive methods for treating urinary incontinence or used in treating urinary incontinence, e.g., bimagrumab, include about 1 mg/kg body weight or about 3 mg/kg body weight or about 10 mg/kg body weight, once per week by subcutaneous administration.

In some methods, two or more monoclonal antibodies with different binding specificities are comprised in the compositions of the disclosure and, thus, administered simultaneously, in which case the dosage of each antibody administered falls within the ranges indicated. An antibody is usually administered on multiple occasions. Intervals between single dosages can be, for example, weekly, monthly, every three months, every six months or yearly. Intervals can also be irregular as indicated by measuring blood levels of antibody to the target antigen in the patient. In some methods, dosage is adjusted to achieve a plasma antibody concentration of about 1-about 1000 μg/ml and in some methods about 25-about 300 μg/ml. For example, an ActRII antibody could be co-administered with an anti-myostatin antibody.

Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Administration of a “therapeutically effective dosage” of an anti-ActRII antibody comprised in the compositions for use in the inventive methods for treating urinary incontinence or used in treating urinary incontinence can result in a decrease in severity of disease symptoms, an increase in frequency and duration of disease symptom-free periods, or a prevention of impairment or disability due to the disease affliction ie. an increase in continence function. Disease symptoms are (i) incontinence following a sudden cough, sneezing, laughing, heavy lifting and exercise or (ii) involuntary contraction of the muscular wall of the bladder that causes an urge to urinate that cannot be stopped or (iii) bladder cannot hold as much urine as the body is making and/or the bladder cannot empty completely, causing small amounts of urinary leakage (patients experiencing constant “dribbling” of urine from the urethra).

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g. Sustained and Controlled Release Drug Delivery Systems, J. R. Robinson, ed., Marcel Dekker, Inc., New York, 1978.

Therapeutic compositions can be administered with medical devices known in the art.

### Uses and Methods of the Disclosure

The disclosed compositions for use in the inventive methods for treating urinary incontinence or used in treating urinary incontinence and the disclosed antibodies have therapeutic utilities, because they have an impact on the treatment of urinary incontinence or on the amelioration of the condition of patients affected by urinary incontinence or on the reduction of symptoms associated with urinary incontinence.

The term “subject” or “individual” as used herein is intended to refer to humans, in particular to a patient suffering from urinary incontinence.

Hence, the disclosure also relates to methods of treatment in which the herein disclosed compositions or the disclosed ActRII receptor antagonists, e.g., ActRII binding molecules, more preferably antibodies to ActRII, e.g., bimagrumab or BYM338, inhibit, i.e. antagonize, the function of ActRII and thereby resulting in the improvement in various types of urinary incontinence. The disclosure provides a method of preventing and or treating urinary incontinence comprising administering a therapeutically effective amount of an ActRII receptor antagonist, e.g., preferably ActRIIB binding molecule, more preferably an antagonist antibody to ActRIIB, e.g., bimagrumab or BYM338 or the disclosed compositions to the patient.

Examples of ActRII receptor antagonists, e.g., ActRII binding molecules, preferably antagonist antibodies to ActRIIB, e.g., bimagrumab or BYM338, that can be used in the disclosed methods of treatment are those disclosed or described in detail above. In certain embodiments, the ActRII antibodies (e.g., bimagrumab or BYM338) are comprised in the herein disclosed compositions for use in the inventive methods for treating urinary incontinence or used in treating urinary incontinence.

The disclosure also relates to the use of an ActRII receptor antagonist, e.g., ActRIIA or ActRIIB receptor binding molecule, preferably an antagonist antibody to ActRII, e.g., BYM338, in the manufacture of a medicament for treating various forms of urinary incontinence as hereinbefore described.

The ActRII binding molecule, preferably an antagonist antibody to ActRII, e.g., bimagrumab or BYM338, may be administered as the sole active agent or in conjunction with, e.g. as an adjuvant to or in combination to, other drugs e.g. IGF-1 or variants of IGF-1, an anti-myostatin antibody, a myostatin propeptide, a myostatin decoy protein that binds ActRIIB but does not activate it, a beta 2 agonist, a Ghrelin agonist, a SARM, GH agonists/mimetics or follistatin.

In accordance with the foregoing the present disclosure provides in a yet further aspect a method or use as defined above comprising co-administration, e.g. concomitantly or in sequence, of a therapeutically effective amount of an ActRII receptor antagonist, preferably an ActRII binding molecule, more preferably an antagonist antibody to ActRII, e.g., bimagrumab or BYM338, and at least one second drug substance, said second drug substance being IGF-1 or variants of IGF-1, an anti-myostatin antibody, a myostatin propeptide, a myostatin decoy protein that binds ActRII but does not activate it, a beta 2 agonist, a Ghrelin agonist, a SARM, GH agonists/mimetics or follistatin.

### Kits

The invention also encompasses kits for use in the inventive methods for treating urinary incontinence or used in treating urinary incontinence which may comprise an ActRII receptor antagonist, e.g., an ActRII receptor binding molecule (e.g., an ActRII receptor antibody or antigen binding fragment thereof, e.g., bimagrumab or BYM338) or ActRII receptor (i.e., ActRIIB receptor) binding molecule (e.g., anti-ActRIIB antibody or antigen binding fragment thereof) (e.g., in liquid or lyophilized form) or a pharmaceutical composition comprising the ActRII receptor antagonist (described supra). Additionally, such kits may comprise means for administering the ActRII antagonist (e.g., a syringe and vial, a prefilled syringe, a prefilled pen) and instructions for use. These kits may contain additional therapeutic agents (described supra), e.g., for delivery in combination with the enclosed myostatin antagonist, e.g., BYM338.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, etc. With such items, a patient may self-administer the drug (i.e., administer the drug on their own behalf) or a physician may administer the drug.

Each component of the kit is usually enclosed within an individual container, and all of the various containers are within a single package along with instructions for use.

It has been contemplated that ActRII antagonists may be ideal candidates in the treatment of urinary incontinence having therapeutic advantages, such as one or more of the following:
i. Reduced number of incontinence episodes per 24 hours;
ii. Reduced number of micturitions per 24 hours;
iii. Reduced volume voided per micturition/incontinence episode;
iv. Reduced number of urgency incontinence episodes;
v. Reduced number of nocturia episodes per 24 hours;
vi. Reduced number of involuntary leakage of urine accompanied by or immediately proceeded by urgency;
vii. Improvement in Patient Perception of Bladder Condition (PPBC)]

The PPBC scale is a global assessment tool that asks patients to rate their impression of their current bladder condition on a 6-point scale from 1: ‘Does not cause me any problems at all’; 2: ‘Causes me some very minor problems’; 3: ‘Causes me some minor problems’; 4: ‘Causes me (some) moderate problems’; 5: ‘Causes me severe problems’ and 6: ‘Causes me many severe problems’. Improvement can be defined as at least a 1 point improvement from Baseline to post-baseline and a major improvement can be defined as at least a 2 point improvement from Baseline to post-baseline in PPBC score

The skilled person knows how to design controlled trials of nonsurgical treatments for urinary incontinence. A systematic review of 96 randomized, controlled trials (RCTs) of nonsurgical treatments for urinary incontinence was published by Shamliyan and co-workers (Tatyana A. Shamliyan, MD, M S; Robert L. Kane, MD; Jean Wyman, PhD; and Timothy J. Wilt: Systematic Review: Randomized, Controlled Trials of Nonsurgical Treatments for Urinary Incontinence in Women; Mar. 18, 2008 Annals of Internal Medicine Volume 148, Number 6, pages 459 to 474. For example, a clinical trial using the antibody bimagrumab could be designed similar to the study conducted under the ClinicalTrials.gov Identifier NCT00689104: Study to Test the Efficacy and Safety of the Beta-3 Agonist Mirabegron (YM178) in Patients With Symptoms of Overactive Bladder.

### Sequences

TABLE 3
sequence listing

| | Ab | |
|---|---|---|
| SEQ ID NO | region | Sequence |
| SEQ ID NO1 | HCDR1 | GYTFTSSYIN |
| SEQ ID NO2 | HCDR1 | GYTFTSSYIN |
| SEQ ID NO3 | HCDR1 | GYTFTSSYIN |
| SEQ ID NO4 | HCDR1 | GYTFTSSYIN |
| SEQ ID NO5 | HCDR1 | GYTFTSSYIN |
| SEQ ID NO6 | HCDR1 | GYTFTSSYIN |
| SEQ ID NO7 | HCDR1 | GYTFTSSYIN |
| SEQ ID NO8 | HCDR1 | GYTFTSSYIN |
| SEQ ID NO9 | HCDR1 | GYTFTSSYIN |
| SEQ ID NO10 | HCDR1 | GYTFTSSYIN |
| SEQ ID NO11 | HCDR1 | GYTFTSSYIN |
| SEQ ID NO12 | HCDR1 | GYTFTSSYIN |
| SEQ ID NO13 | HCDR1 | GYTFTSSYIN |
| SEQ ID NO14 | HCDR1 | GYTFTSSYIN |
| SEQ ID NO15 | HCDR2 | TINPVSGNTSYAQKFQG |
| SEQ ID NO16 | HCDR2 | TINPVSGNTSYAQKFQG |
| SEQ ID NO17 | HCDR2 | TINPVSGNTSYAQKFQG |
| SEQ ID NO18 | HCDR2 | TINPVSGNTSYAQKFQG |
| SEQ ID NO19 | HCDR2 | MINAPIGTTRYAQKFQG |
| SEQ ID NO20 | HCDR2 | QINAASGMTRYAQKFQG |
| SEQ ID NO21 | HCDR2 | MINAPIGTTRYAQKFQG |
| SEQ ID NO22 | HCDR2 | TINPVSGNTRYAQKFQG |
| SEQ ID NO23 | HCDR2 | TINPVSGSTSYAQKFQG |
| SEQ ID NO24 | HCDR2 | QINAASGMTRYAQKFQG |
| SEQ ID NO25 | HCDR2 | NINAAAGITLYAQKFQG |
| SEQ ID NO26 | HCDR2 | TINPPTGGTYYAQKFQG |
| SEQ ID NO27 | HCDR2 | GINPPAGTTSYAQKFQG |
| SEQ ID NO28 | HCDR2 | NINPATGHADYAQKFQG |
| SEQ ID NO29 | HCDR3 | GGWFDY |
| SEQ ID NO30 | HCDR3 | GGWFDY |
| SEQ ID NO31 | HCDR3 | GGWFDY |
| SEQ ID NO32 | HCDR3 | GGWFDY |
| SEQ ID NO33 | HCDR3 | GGWFDY |
| SEQ ID NO34 | HCDR3 | GGWFDY |
| SEQ ID NO35 | HCDR3 | GGWFDY |
| SEQ ID NO36 | HCDR3 | GGWFDY |
| SEQ ID NO37 | HCDR3 | GGWFDY |
| SEQ ID NO38 | HCDR3 | GGWFDY |
| SEQ ID NO39 | HCDR3 | GGWFDY |
| SEQ ID NO40 | HCDR3 | GGWFDY |
| SEQ ID NO41 | HCDR3 | GGWFDY |
| SEQ ID NO42 | HCDR3 | GGWFDY |
| SEQ ID NO43 | LCDR1 | TGTSSDVGSYNYVN |
| SEQ ID NO44 | LCDR1 | TGTSSDVGSYNYVN |
| SEQ ID NO45 | LCDR1 | TGTSSDVGSYNYVN |
| SEQ ID NO46 | LCDR1 | TGTSSDVGSYNYVN |
| SEQ ID NO47 | LCDR1 | TGTSSDVGSYNYVN |
| SEQ ID NO48 | LCDR1 | TGTSSDVGSYNYVN |
| SEQ ID NO49 | LCDR1 | TGTSSDVGSYNYVN |
| SEQ ID NO50 | LCDR1 | TGTSSDVGSYNYVN |
| SEQ ID NO51 | LCDR1 | TGTSSDVGSYNYVN |
| SEQ ID NO52 | LCDR1 | TGTSSDVGSYNYVN |
| SEQ ID NO53 | LCDR1 | TGTSSDVGSYNYVN |
| SEQ ID NO54 | LCDR1 | TGTSSDVGSYNYVN |
| SEQ ID NO55 | LCDR1 | TGTSSDVGSYNYVN |
| SEQ ID NO56 | LCDR1 | TGTSSDVGSYNYVN |
| SEQ ID NO57 | LDCR2 | LMIYGVSKRPS |
| SEQ ID NO58 | LDCR2 | LMIYGVSKRPS |
| SEQ ID NO59 | LDCR2 | LMIYGVSKRPS |
| SEQ ID NO60 | LDCR2 | LMIYGVSKRPS |
| SEQ ID NO61 | LDCR2 | LMIYGVSKRPS |
| SEQ ID NO62 | LDCR2 | LMIYGVSKRPS |
| SEQ ID NO63 | LDCR2 | LMIYGVSKRPS |
| SEQ ID NO64 | LDCR2 | LMIYGVSKRPS |
| SEQ ID NO65 | LDCR2 | LMIYGVSKRPS |
| SEQ ID NO66 | LDCR2 | LMIYGVSKRPS |
| SEQ ID NO67 | LDCR2 | LMIYGVSKRPS |
| SEQ ID NO68 | LDCR2 | LMIYGVSKRPS |
| SEQ ID NO69 | LDCR2 | LMIYGVSKRPS |
| SEQ ID NO70 | LDCR2 | LMIYGVSKRPS |
| SEQ ID NO71 | LCDR3 | QAWTSKMAG |
| SEQ ID NO72 | LCDR3 | SSYTRMGHP |
| SEQ ID NO73 | LCDR3 | ATYGKGVTPP |
| SEQ ID NO74 | LCDR3 | GTFAGGSYYG |
| SEQ ID NO75 | LCDR3 | QAWTSKMAG |
| SEQ ID NO76 | LCDR3 | QAWTSKMAG |
| SEQ ID NO77 | LCDR3 | GTFAGGSYYG |
| SEQ ID NO78 | LCDR3 | GTFAGGSYYG |
| SEQ ID NO79 | LCDR3 | GTFAGGSYYG |
| SEQ ID NO80 | LCDR3 | GTFAGGSYYG |
| SEQ ID NO81 | LCDR3 | GTFAGGSYYG |
| SEQ ID NO82 | LCDR3 | GTFAGGSYYG |
| SEQ ID NO83 | LCDR3 | GTFAGGSYYG |
| SEQ ID NO84 | LCDR3 | GTFAGGSYYG |
| SEQ ID NO85 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCQAWTSKMAGVFGGGTKLTVLGQ |
| SEQ ID NO86 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCSSYTRMGHPVFGGGTKLTVLGQ |
| SEQ ID NO87 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCATYGKGVTPPVFGGGTKLTVLGQ |
| SEQ ID NO88 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO89 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCQAWTSKMAGVFGGGTKLTVLGQ |
| SEQ ID NO90 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCQAWTSKMAGVFGGGTKLTVLGQ |
| SEQ ID NO91 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO92 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO93 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO94 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO95 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO96 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO97 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO98 | VL | DIALTQPASVSGSPGQSITISCTGTSSDVGSYNYVNWYQQHPGKAPKLMIYGVSKRPSGV |
| | | SNRFSGSKSGNTASLTISGLQAEDEADYYCGTFAGGSYYGVFGGGTKLTVLGQ |
| SEQ ID NO99 | VH | QVQLVQSGAEVKKPGASVKVSCKASGYTFTSSYINWVRQAPGQGLEWMGTINPVSGNT |
| | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPVSGNT |
| NO100 | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPVSGNT |
| NO101 | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPVSGNT |
| NO102 | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGMINAPIGTTR |
| NO103 | | YAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGQINAASGMT |
| NO104 | | RYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGMINAPIGTTR |
| NO105 | | YAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPVSGNT |
| NO106 | | RYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPVSGST |
| NO107 | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGQINAASGMT |
| NO108 | | RYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGNINAAAGITL |
| NO109 | | YAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGTINPPTGGT |
| NO110 | | YYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGGINPPAGTT |
| NO111 | | SYAQKFQGRVTMTRDTSISTAYMELSSLRSEDTAVYYCARGGWFDYWGQGTLVTVSS |
| SEQ ID | VH | QVQLVQSGAEVKKPGASVKVSCKASGYTFTSSYINWVRQAPGQGLEWMGNINPATGHA |
| NO112 | | DYAQKFQGRVTMTRDTSISTAYMELSSLRSEDTAVYYCARGGWFDYWGQGTLVTVSS |
| SEQ ID | DNA VL | GATATCGCACTGACCCAGCCAGCTTCAGTGAGCGGCTCACCAGGTCAGAGCATTACC |
| NO113 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCCAGGCTTGGACTTCT |
| | | AAGATGGCTGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO114 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCTCTTCTTATACTCGTA |
| | | TGGGTCATCCTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO115 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGCTACTTATGGTAAG |
| | | GGTGTTACTCCTCCTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO116 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO117 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCCAGGCTTGGACTTCT |
| | | AAGATGGCTGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO118 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCCAGGCTTGGACTTCT |
| | | AAGATGGCTGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO119 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO120 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO121 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO122 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO123 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO124 | | ATCTCGTGTACTGGTACTAGCAGCGATGTTGGTTCTTATAATTATGTGAATTGGTACCA |
| | | GCAGCATCCCGGGAAGGCGCCGAAACTTATGATTTATGGTGTTTCTAAGCGTCCCTC |
| | | AGGCGTGAGCAACCGTTTTAGCGGATCCAAAAGCGGCAACACCGCGAGCCTGACCAT |
| | | TAGCGGCCTGCAAGCGGAAGACGAAGCGGATTATTATTGCGGTACTTTTGCTGGTGG |
| | | TTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO125 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VL | GATATCGCACTGACCCAGCCAGCTTCAGTGAGCGGCTCACCAGGTCAGAGCATTACC |
| NO126 | | ATCTCGTGTACGGGTACTAGCAGCGATGTTGGTTCTTATAATTATGTGAATTGGTACC |
| | | AGCAGCATCCCGGGAAGGCGCCGAAACTTATGATTTATGGTGTTTCTAAGCGTCCCT |
| | | CAGGCGTGAGCAACCGTTTTAGCGGATCCAAAAGCGGCAACACCGCGAGCCTGACC |
| | | ATTAGCGGCCTGCAAGCGGAAGACGAAGCGGATTATTATTGCGGTACTTTTGCTGGT |
| | | GGTTCTTATTATGGTGTGTTTGGCGGCGGCACGAAGTTAACCGTTCTTGGCCAG |
| SEQ ID | DNA VH | CAGGTGCAATTGGTTCAGAGCGGCGCGGAAGTGAAAAAACCGGGCGCGAGCGTGAA |
| NO127 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCAATA |
| | | CGTCTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO128 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCAATA |
| | | CGTCTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO129 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCAATA |
| | | CGTCTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO130 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCAATA |
| | | CGTCTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO131 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCATGATTAATGCTCCTATTGGTACTA |
| | | CTCGTTATGCTCAGAAGTTTCAGGGTCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO132 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCCAGATTAATGCTGCTTCTGGTATGA |
| | | CTCGTTATGCTCAGAAGTTTCAGGGTCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO133 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCATGATTAATGCTCCTATTGGTACTA |
| | | CTCGTTATGCTCAGAAGTTTCAGGGTCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO134 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCAATA |
| | | CGCGTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO135 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATCAATCCGGTTTCTGGCTCTA |
| | | CGTCTTACGCGCAGAAGTTTCAGGGCCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO136 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCCAGATTAATGCTGCTTCTGGTATGA |
| | | CTCGTTATGCTCAGAAGTTTCAGGGTCGGGTCACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | DNA VH | CAGGTGCAATTGGTTCAGAGCGGCGCGGAAGTGAAAAAACCGGGCGCGAGCGTGAA |
| NO137 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCAATATTAATGCTGCTGCTGGTATTA |
| | | CTCTTTATGCTCAGAAGTTTCAGGGTCGGGTCACCATGACCCGTGATACCAGCATTAG |
| | | CACCGCGTATATGGAACTGAGCAGCCTGCGTAGCGAAGATACGGCCGTGTATTATTG |
| | | CGCGCGTGGTGGTTGGTTTGATTATTGGGGCCAAGGCACCCTGGTGACGGTTAGCTC |
| | | A |
| SEQ ID | DNA VH | CAGGTGCAATTGGTTCAGAGCGGCGCGGAAGTGAAAAAACCGGGCGCGAGCGTGAA |
| NO138 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCACTATTAATCCTCCTACTGGAGGTA |
| | | CTTATTATGCTCAGAAGTTTCAGGGTCGGGTGACCATGACCCGTGATACCAGCATTAG |
| | | CACCGCGTATATGGAACTGAGCAGCCTGCGTAGCGAAGATACGGCCGTGTATTATTG |
| | | CGCGCGTGGTGGTTGGTTTGATTATTGGGGCCAAGGCACCCTGGTGACGGTTAGCTC |
| | | A |
| SEQ ID | DNA VH | CAGGTGCAATTGGTTCAGAGCGGCGCGGAAGTGAAAAAACCGGGCGCGAGCGTGAA |
| NO139 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCGGTATTAATCCTCCTGCTGGTACTA |
| | | CTTCTTATGCTCAGAAGTTTCAGGGTCGGGTCACCATGACCCGTGATACCAGCATTAG |
| | | CACCGCGTATATGGAACTGAGCAGCCTGCGTAGCGAAGATACGGCCGTGTATTATTG |
| | | CGCGCGTGGTGGTTGGTTTGATTATTGGGGCCAAGGCACCCTGGTGACGGTTAGCTC |
| | | A |
| SEQ ID | DNA VH | CAGGTGCAATTGGTTCAGAGCGGCGCGGAAGTGAAAAAACCGGGCGCGAGCGTGAA |
| NO140 | | AGTGAGCTGCAAAGCCTCCGGATATACCTTTACTTCTTCTTATATTAATTGGGTCCGCC |
| | | AAGCCCCTGGGCAGGGTCTCGAGTGGATGGGCAATATTAATCCTGCTACTGGTCATG |
| | | CTGATTATGCTCAGAAGTTTCAGGGTCGGGTGACCATGACCCGTGATACCAGCATTA |
| | | GCACCGCGTATATGGAACTGAGCAGCCTGCGTAGCGAAGATACGGCCGTGTATTATT |
| | | GCGCGCGTGGTGGTTGGTTTGATTATTGGGGCCAAGGCACCCTGGTGACGGTTAGCT |
| | | CA |
| SEQ ID | Light | QSALTQPASVSGSPGQSITISCTGTSSDVGSYNYVNWYQQHPGKAPKLMIYGVSKRPSG |
| NO141 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO142 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO143 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO144 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO145 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Heavy | QVQLVQSGAEVKKPGASVKVSCKASGYTFTSSYINWVRQAPGQGLEWMGTINPVSGST |
| NO146 | Chain | SYAQKFQGRVTMTRDTSISTAYMELSRLRSDDTAVYYCARGGWFDYWGQGTLVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAAGG |
| | | PSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ ID | Heavy | QVQLVQSGAEVKKPGASVKVSCKASGYTFTSSYINWVRQAPGQGLEWMGQINAASGMT |
| NO147 | Chain | RYAQKFQGRVTMTRDTSISTAYMELSRLRSDDTAVYYCARGGWFDYWGQGTLVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAAGG |
| | | PSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ ID | Heavy | QVQLVQSGAEVKKPGASVKVSCKASGYTFTSSYINWVRQAPGQGLEWMGNINAAAGITL |
| NO148 | Chain | YAQKFQGRVTMTRDTSISTAYMELSRLRSDDTAVYYCARGGWFDYWGQGTLVTVSSAS |
| | | TKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKRVEPKSCDKTHTCPPCPAPEAAGGPS |
| | | VFLFPPKPKDTLMISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQVYTLPPSREEMT |
| | | KNQVSLTCLVKGFYPSDIAVEWESNGQPENNYKTTPPVLDSDGSFFLYSKLTVDKSRWQ |
| | | QGNVFSCSVMHEALHNHYTQKSLSLSPGK |
| SEQ ID | Heavy | QVQLVQSGAEVKKPGASVKVSCKASGYTFTSSYINWVRQAPGQGLEWMGGINPPAGTT |
| NO149 | Chain | SYAQKFQGRVTMTRDTSISTAYMELSRLRSDDTAVYYCARGGWFDYWGQGTLVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAAGG |
| | | PSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ ID | Heavy | QVQLVQSGAEVKKPGASVKVSCKASGYTFTSSYINWVRQAPGQGLEWMGNINPATGHA |
| NO150 | Chain | DYAQKFQGRVTMTRDTSISTAYMELSRLRSDDTAVYYCARGGWFDYWGQGTLVTVSSA |
| | | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTHTCPPCPAPEAAGG |
| | | PSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGSFFLYSKLTVDKSR |
| | | WQQGNVFSCSVMHEALHNHYTQKSLSLSPGK |
| SEQ ID | Light | QSALTQPASVSGSPGQSITISCTGTSSDVGSYNYVNWYQQHPGKAPKLMIYGVSKRPSG |
| NO151 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO152 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO153 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO154 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Light | QSALTQPASVSGSPGQSITISCTGTSSDVGSYNYVNWYQQHPGKAPKLMIYGVSKRPSG |
| NO155 | Chain | VSNRFSGSKSGNTASLTISGLQAEDEADYYCGTFAGGSYYGVFGGGTKLTVLGQPKAAP |
| | | SVTLFPPSSEELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| SEQ ID | Heavy | QVQLVQSGAEVKKPGASVKVSCKASGYTFTSSYINWVRQAPGQGLEWMGTINPVSGST |
| NO156 | Chain | SYAQKFQGRVTMTRDTSISTAYMELSRLRSDDTAVYYCARGGWFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCVVVDVSHEDPEVQFNWYVDGVEVHNAKTKPREEQFNSTFR |
| | | VVSVLTVVHQDWLNGKEYKCKVSNKGLPAPIEKTISKTKGQPREPQVYTLPPSREEMTKN |
| | | QVSLTCLVKGFYPSDIAVEWESNGQPENNYKTTPPMLDSDGSFFLYSKLTVDKSRWQQG |
| | | NVFSCSVMHEALHNHYTQKSLSLSPGK |
| SEQ ID | Heavy | QVQLVQSGAEVKKPGASVKVSCKASGYTFTSSYINWVRQAPGQGLEWMGQINAASGMT |
| NO157 | Chain | RYAQKFQGRVTMTRDTSISTAYMELSRLRSDDTAVYYCARGGWFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCVVVDVSHEDPEVQFNWYVDGVEVHNAKTKPREEQFNSTFR |
| | | VVSVLTVVHQDWLNGKEYKCKVSNKGLPAPIEKTISKTKGQPREPQVYTLPPSREEMTKN |
| | | QVSLTCLVKGFYPSDIAVEWESNGQPENNYKTTPPMLDSDGSFFLYSKLTVDKSRWQQG |
| | | NVFSCSVMHEALHNHYTQKSLSLSPGK |
| SEQ ID | Heavy | QVQLVQSGAEVKKPGASVKVSCKASGYTFTSSYINWVRQAPGQGLEWMGNINAAAGITL |
| NO158 | Chain | YAQKFQGRVTMTRDTSISTAYMELSRLRSDDTAVYYCARGGWFDYWGQGTLVTVSSAS |
| | | TKGPSVFPLAPCSRSTSESTAALGCLVKDYFPEPVTVSWNSGALTSGVHTFPAVLQSSGL |
| | | YSLSSVVTVPSSNFGTQTYTCNVDHKPSNTKVDKTVERKCCVECPPCPAPPVAGPSVFLF |
| | | PPKPKDTLMISRTPEVTCVVVDVSHEDPEVQFNWYVDGVEVHNAKTKPREEQFNSTFRV |
| | | VSVLTVVHQDWLNGKEYKCKVSNKGLPAPIEKTISKTKGQPREPQVYTLPPSREEMTKNQ |
| | | VSLTCLVKGFYPSDIAVEWESNGQPENNYKTTPPMLDSDGSFFLYSKLTVDKSRWQQGN |
| | | VFSCSVMHEALHNHYTQKSLSLSPGK |
| SEQ ID | Heavy | QVQLVQSGAEVKKPGASVKVSCKASGYTFTSSYINWVRQAPGQGLEWMGGINPPAGTT |
| NO159 | Chain | SYAQKFQGRVTMTRDTSISTAYMELSRLRSDDTAVYYCARGGWFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCVVVDVSHEDPEVQFNWYVDGVEVHNAKTKPREEQFNSTFR |
| | | VVSVLTVVHQDWLNGKEYKCKVSNKGLPAPIEKTISKTKGQPREPQVYTLPPSREEMTKN |
| | | QVSLTCLVKGFYPSDIAVEWESNGQPENNYKTTPPMLDSDGSFFLYSKLTVDKSRWQQG |
| | | NVFSCSVMHEALHNHYTQKSLSLSPGK |
| SEQ ID | Heavy | QVQLVQSGAEVKKPGASVKVSCKASGYTFTSSYINWVRQAPGQGLEWMGNINPATGHA |
| NO160 | Chain | DYAQKFQGRVTMTRDTSISTAYMELSRLRSDDTAVYYCARGGWFDYWGQGTLVTVSSA |
| | | STKGPSVFPLAPCSRSTSESTAALGCLVKDYFPEPVTVSWNSGALTSGVHTFPAVLQSSG |
| | | LYSLSSVVTVPSSNFGTQTYTCNVDHKPSNTKVDKTVERKCCVECPPCPAPPVAGPSVFL |
| | | FPPKPKDTLMISRTPEVTCVVVDVSHEDPEVQFNWYVDGVEVHNAKTKPREEQFNSTFR |
| | | VVSVLTVVHQDWLNGKEYKCKVSNKGLPAPIEKTISKTKGQPREPQVYTLPPSREEMTKN |
| | | QVSLTCLVKGFYPSDIAVEWESNGQPENNYKTTPPMLDSDGSFFLYSKLTVDKSRWQQG |
| | | NVFSCSVMHEALHNHYTQKSLSLSPGK |
| SEQ ID | DNA | CAGAGCGCCCTGACCCAGCCCGCCAGCGTGTCCGGCAGCCCAGGCCAGTCTATCAC |
| NO161 | Light | AATCAGCTGCACCGGCACCTCCAGCGACGTGGGCAGCTACAACTACGTGAACTGGTA |
| | Chain | TCAGCAGCACCCCGGCAAGGCCCCCAAGCTGATGATCTACGGCGTGAGCAAGAGGC |
| | | CCAGCGGCGTGTCCAACAGGTTCAGCGGCAGCAAGAGCGGCAACACCGCCAGCCTG |
| | | ACAATCAGTGGGCTGCAGGCTGAGGACGAGGCCGACTACTACTGCGGCACCTTTGC |
| | | CGGCGGATCATACTACGGCGTGTTCGGCGGAGGGACCAAGCTGACCGTGCTGGGCC |
| | | AGCCTAAGGCTGCCCCCAGCGTGACCCTGTTCCCCCCCAGCAGCGAGGAGCTGCAG |
| | | GCCAACAAGGCCACCCTGGTGTGCCTGATCAGCGACTTCTACCCAGGCGCCGTGAC |
| | | CGTGGCCTGGAAGGCCGACAGCAGCCCCGTGAAGGCCGGCGTGGAGACCACCACC |
| | | CCCAGCAAGCAGAGCAACAACAAGTACGCCGCCAGCAGCTACCTGAGCCTGACCCC |
| | | CGAGCAGTGGAAGAGCCACAGGTCCTACAGCTGCCAGGTGACCCACGAGGGCAGCA |
| | | CCGTGGAAAAGACCGTGGCCCCAACCGAGTGCAGC |
| SEQ ID | DNA | CAGAGCGCCCTGACCCAGCCCGCCAGCGTGTCCGGCAGCCCAGGCCAGTCTATCAC |
| NO162 | Light | AATCAGCTGCACCGGCACCTCCAGCGACGTGGGCAGCTACAACTACGTGAACTGGTA |
| | Chain | TCAGCAGCACCCCGGCAAGGCCCCCAAGCTGATGATCTACGGCGTGAGCAAGAGGC |
| | | CCAGCGGCGTGTCCAACAGGTTCAGCGGCAGCAAGAGCGGCAACACCGCCAGCCTG |
| | | ACAATCAGTGGGCTGCAGGCTGAGGACGAGGCCGACTACTACTGCGGCACCTTTGC |
| | | CGGCGGATCATACTACGGCGTGTTCGGCGGAGGGACCAAGCTGACCGTGCTGGGCC |
| | | AGCCTAAGGCTGCCCCCAGCGTGACCCTGTTCCCCCCCAGCAGCGAGGAGCTGCAG |
| | | GCCAACAAGGCCACCCTGGTGTGCCTGATCAGCGACTTCTACCCAGGCGCCGTGAC |
| | | CGTGGCCTGGAAGGCCGACAGCAGCCCCGTGAAGGCCGGCGTGGAGACCACCACC |
| | | CCCAGCAAGCAGAGCAACAACAAGTACGCCGCCAGCAGCTACCTGAGCCTGACCCC |
| | | CGAGCAGTGGAAGAGCCACAGGTCCTACAGCTGCCAGGTGACCCACGAGGGCAGCA |
| | | CCGTGGAAAAGACCGTGGCCCCAACCGAGTGCAGC |
| SEQ ID | DNA | CAGAGCGCACTGACCCAGCCAGCTTCAGTGAGCGGCTCACCAGGTCAGAGCATTAC |
| NO163 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGAGCGCACTGACCCAGCCAGCTTCAGTGAGCGGCTCACCAGGTCAGAGCATTAC |
| NO164 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGAGCGCACTGACCCAGCCAGCTTCAGTGAGCGGCTCACCAGGTCAGAGCATTAC |
| NO165 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGGTGCAGCTGGTGCAGAGCGGAGCTGAGGTGAAGAAGCCAGGCGCCAGCGTCAA |
| NO166 | Heavy | GGTGTCCTGCAAGGCCAGCGGCTACACCTTCACCAGCAGCTACATCAACTGGGTCCG |
| | Chain | CCAGGCTCCTGGGCAGGGACTGGAGTGGATGGGCACCATCAACCCCGTGTCCGGCA |
| | | GCACCAGCTACGCCCAGAAGTTCCAGGGCAGAGTCACCATGACCAGGGACACCAGC |
| | | ATCAGCACCGCCTACATGGAGCTGTCCAGGCTGAGAAGCGACGACACCGCCGTGTA |
| | | CTACTGCGCCAGGGGCGGCTGGTTCGACTACTGGGGCCAGGGCACCCTGGTGACCG |
| | | TGTCCTCAGCTAGCACCAAGGGCCCCAGCGTGTTCCCCCTGGCCCCCAGCAGCAAG |
| | | AGCACCTCCGGCGGCACAGCCGCCCTGGGCTGCCTGGTGAAGGACTACTTCCCCGA |
| | | GCCCGTGACCGTGTCCTGGAACAGCGGAGCCCTGACCAGCGGCGTGCACACCTTCC |
| | | CCGCCGTGCTGCAGAGCAGCGGCCTGTACAGCCTGTCCAGCGTGGTGACAGTGCCC |
| | | AGCAGCAGCCTGGGCACCCAGACCTACATCTGCAACGTGAACCACAAGCCCAGCAAC |
| | | ACCAAGGTGGACAAGAGAGTGGAGCCCAAGAGCTGCGACAAGACCCACACCTGCCC |
| | | CCCCTGCCCAGCCCCCGAAGCTGCAGGCGGCCCTTCCGTGTTCCTGTTCCCCCCCA |
| | | AGCCCAAGGACACCCTGATGATCAGCAGGACCCCCGAGGTGACCTGCGTGGTGGTG |
| | | GACGTGAGCCACGAGGACCCAGAGGTGAAGTTCAACTGGTACGTGGACGGCGTGGA |
| | | GGTGCACAACGCCAAGACCAAGCCCAGAGAGGAGCAGTACAACAGCACCTACAGGG |
| | | TGGTGTCCGTGCTGACCGTGCTGCACCAGGACTGGCTGAACGGCAAAGAATACAAGT |
| | | GCAAGGTCTCCAACAAGGCCCTGCCTGCCCCCATCGAAAAGACCATCAGCAAGGCCA |
| | | AGGGCCAGCCACGGGAGCCCCAGGTGTACACCCTGCCCCCTTCTCGGGAGGAGATG |
| | | ACCAAGAACCAGGTGTCCCTGACCTGTCTGGTGAAGGGCTTCTACCCCAGCGACATC |
| | | GCCGTGGAGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGGACAAGAG |
| | | CAGGTGGCAGCAGGGCAACGTGTTCAGCTGCAGCGTGATGCACGAGGCCCTGCACA |
| | | ACCACTACACCCAGAAGAGCCTGAGCCTGTCACCCGGCAAG |
| SEQ ID | DNA | CAGGTGCAGCTGGTGCAGAGCGGAGCTGAGGTGAAGAAGCCAGGCGCCAGCGTCAA |
| NO167 | Heavy | GGTGTCCTGCAAGGCCAGCGGCTACACCTTCACCAGCAGCTACATCAACTGGGTGCG |
| | Chain | CCAGGCTCCAGGGCAGGGACTGGAGTGGATGGGCCAGATCAACGCCGCCAGCGGC |
| | | ATGACCAGATACGCCCAGAAGTTCCAGGGCAGAGTCACAATGACCAGGGACACCTCT |
| | | ATCAGCACCGCCTACATGGAGCTGTCCAGGCTGAGAAGCGACGACACCGCCGTGTA |
| | | CTACTGCGCCAGGGGCGGCTGGTTCGACTACTGGGGCCAGGGCACCCTGGTGACCG |
| | | TGTCCTCAGCTAGCACCAAGGGCCCCAGCGTGTTCCCCCTGGCCCCCAGCAGCAAG |
| | | AGCACCTCCGGCGGCACAGCCGCCCTGGGCTGCCTGGTGAAGGACTACTTCCCCGA |
| | | GCCCGTGACCGTGTCCTGGAACAGCGGAGCCCTGACCAGCGGCGTGCACACCTTCC |
| | | CCGCCGTGCTGCAGAGCAGCGGCCTGTACAGCCTGTCCAGCGTGGTGACAGTGCCC |
| | | AGCAGCAGCCTGGGCACCCAGACCTACATCTGCAACGTGAACCACAAGCCCAGCAAC |
| | | ACCAAGGTGGACAAGAGAGTGGAGCCCAAGAGCTGCGACAAGACCCACACCTGCCC |
| | | CCCCTGCCCAGCCCCCGAAGCTGCAGGCGGCCCTTCCGTGTTCCTGTTCCCCCCCA |
| | | AGCCCAAGGACACCCTGATGATCAGCAGGACCCCCGAGGTGACCTGCGTGGTGGTG |
| | | GACGTGAGCCACGAGGACCCAGAGGTGAAGTTCAACTGGTACGTGGACGGCGTGGA |
| | | GGTGCACAACGCCAAGACCAAGCCCAGAGAGGAGCAGTACAACAGCACCTACAGGG |
| | | TGGTGTCCGTGCTGACCGTGCTGCACCAGGACTGGCTGAACGGCAAAGAATACAAGT |
| | | GCAAGGTCTCCAACAAGGCCCTGCCTGCCCCCATCGAAAAGACCATCAGCAAGGCCA |
| | | AGGGCCAGCCACGGGAGCCCCAGGTGTACACCCTGCCCCCTTCTCGGGAGGAGATG |
| | | ACCAAGAACCAGGTGTCCCTGACCTGTCTGGTGAAGGGCTTCTACCCCAGCGACATC |
| | | GCCGTGGAGTGGGAGAGCAACGGCCAGCCCGAGAACAACTACAAGACCACCCCCCC |
| | | AGTGCTGGACAGCGACGGCAGCTTCTTCCTGTACAGCAAGCTGACCGTGGACAAGAG |
| | | CAGGTGGCAGCAGGGCAACGTGTTCAGCTGCAGCGTGATGCACGAGGCCCTGCACA |
| | | ACCACTACACCCAGAAGAGCCTGAGCCTGTCACCCGGCAAG |
| SEQ ID | DNA | CAGGTGCAATTGGTTCAGAGCGGCGCGGAAGTGAAAAAACCGGGCGCGAGCGTGAA |
| NO168 | Heavy | AGTGAGCTGCAAAGCCTCCGGATATACCTTTACTTCTTCTTATATTAATTGGGTCCGCC |
| | Chain | AAGCCCCTGGGCAGGGTCTCGAGTGGATGGGCAATATTAATGCTGCTGCTGGTATTA |
| | | CTCTTTATGCTCAGAAGTTTCAGGGTCGGGTCACCATGACCCGTGATACCAGCATTAG |
| | | CACCGCGTATATGGAACTGAGCCGCCTGCGTAGCGATGATACGGCCGTGTATTATTG |
| | | CGCGCGTGGTGGTTGGTTTGATTATTGGGGCCAAGGCACCCTGGTGACGGTTAGCTC |
| | | AGCCTCCACCAAGGGTCCATCGGTCTTCCCCCTGGCACCCTCCTCCAAGAGCACCTC |
| | | TGGGGGCACAGCGGCCCTGGGCTGCCTGGTCAAGGACTACTTCCCCGAACCGGTGA |
| | | CGGTGTCGTGGAACTCAGGCGCCCTGACCAGCGGCGTGCACACCTTCCCGGCTGTC |
| | | CTACAGTCCTCAGGACTCTACTCCCTCAGCAGCGTGGTGACCGTGCCCTCCAGCAGC |
| | | TTGGGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGCCCA |
| | | GCACCTGAAGCAGCGGGGGGACCGTCAGTCTTCCTCTTCCCCCCAAAACCCAAGGA |
| | | CACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATG |
| | | CCAAGACAAAGCCGCGGGAGGAGCAGTACAACAGCACGTACCGGGTGGTCAGCGTC |
| | | CTCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCCC |
| | | CGAGAACCACAGGTGTACACCCTGCCCCCATCCCGGGAGGAGATGACCAAGAACCA |
| | | GGTCAGCCTGACCTGCCTGGTCAAAGGCTTCTATCCCAGCGACATCGCCGTGGAGTG |
| | | GGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGC |
| | | AGGGGAACGTCTTCTCATGCTCCGTGATGCATGAGGCTCTGCACAACCACTACACGC |
| | | AGAAGAGCCTCTCCCTGTCTCCGGGTAAA |
| SEQ ID | DNA | CAGGTGCAATTGGTTCAGAGCGGCGCGGAAGTGAAAAAACCGGGCGCGAGCGTGAA |
| NO169 | Heavy | AGTGAGCTGCAAAGCCTCCGGATATACCTTTACTTCTTCTTATATTAATTGGGTCCGCC |
| | Chain | AAGCCCCTGGGCAGGGTCTCGAGTGGATGGGCGGTATTAATCCTCCTGCTGGTACTA |
| | | CTTCTTATGCTCAGAAGTTTCAGGGTCGGGTCACCATGACCCGTGATACCAGCATTAG |
| | | CACCGCGTATATGGAACTGAGCCGCCTGCGTAGCGATGATACGGCCGTGTATTATTG |
| | | CGCGCGTGGTGGTTGGTTTGATTATTGGGGCCAAGGCACCCTGGTGACGGTTAGCTC |
| | | AGCCTCCACCAAGGGTCCATCGGTCTTCCCCCTGGCACCCTCCTCCAAGAGCACCTC |
| | | TGGGGGCACAGCGGCCCTGGGCTGCCTGGTCAAGGACTACTTCCCCGAACCGGTGA |
| | | CGGTGTCGTGGAACTCAGGCGCCCTGACCAGCGGCGTGCACACCTTCCCGGCTGTC |
| | | CTACAGTCCTCAGGACTCTACTCCCTCAGCAGCGTGGTGACCGTGCCCTCCAGCAGC |
| | | TTGGGCACCCAGACCTACATCTGCAACGTGAATCACAAGCCCAGCAACACCAAGGTG |
| | | GACAAGAGAGTTGAGCCCAAATCTTGTGACAAAACTCACACATGCCCACCGTGCCCA |
| | | GCACCTGAAGCAGCGGGGGGACCGTCAGTCTTCCTCTTCCCCCCAAAACCCAAGGA |
| | | CACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATG |
| | | CCAAGACAAAGCCGCGGGAGGAGCAGTACAACAGCACGTACCGGGTGGTCAGCGTC |
| | | CTCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCCC |
| | | CGAGAACCACAGGTGTACACCCTGCCCCCATCCCGGGAGGAGATGACCAAGAACCA |
| | | GGTCAGCCTGACCTGCCTGGTCAAAGGCTTCTATCCCAGCGACATCGCCGTGGAGTG |
| | | GGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGC |
| | | AGGGGAACGTCTTCTCATGCTCCGTGATGCATGAGGCTCTGCACAACCACTACACGC |
| | | AGAAGAGCCTCTCCCTGTCTCCGGGTAAA |
| SEQ ID | DNA | CAGGTGCAATTGGTTCAGAGCGGCGCGGAAGTGAAAAAACCGGGCGCGAGCGTGAA |
| NO170 | Heavy | AGTGAGCTGCAAAGCCTCCGGATATACCTTTACTTCTTCTTATATTAATTGGGTCCGCC |
| | Chain | AAGCCCCTGGGCAGGGTCTCGAGTGGATGGGCAATATTAATCCTGCTACTGGTCATG |
| | | CTGATTATGCTCAGAAGTTTCAGGGTCGGGTGACCATGACCCGTGATACCAGCATTA |
| | | GCACCGCGTATATGGAACTGAGCCGCCTGCGTAGCGATGATACGGCCGTGTATTATT |
| | | GCGCGCGTGGTGGTTGGTTTGATTATTGGGGCCAAGGCACCCTGGTGACGGTTAGCT |
| | | CAGCCTCCACCAAGGGTCCATCGGTCTTCCCCCTGGCACCCTCCTCCAAGAGCACCT |
| | | CTGGGGGCACAGCGGCCCTGGGCTGCCTGGTCAAGGACTACTTCCCCGAACCGGTG |
| | | ACGGTGTCGTGGAACTCAGGCGCCCTGACCAGCGGCGTGCACACCTTCCCGGCTGT |
| | | CCTACAGTCCTCAGGACTCTACTCCCTCAGCAGCGTGGTGACCGTGCCCTCCAGCAG |
| | | CTTGGGCACCCAGACCTACATCTGCAACGTGAATCACAAGCCCAGCAACACCAAGGT |
| | | GGACAAGAGAGTTGAGCCCAAATCTTGTGACAAAACTCACACATGCCCACCGTGCCC |
| | | AGCACCTGAAGCAGCGGGGGGACCGTCAGTCTTCCTCTTCCCCCCAAAACCCAAGGA |
| | | CACCCTCATGATCTCCCGGACCCCTGAGGTCACATGCGTGGTGGTGGACGTGAGCC |
| | | ACGAAGACCCTGAGGTCAAGTTCAACTGGTACGTGGACGGCGTGGAGGTGCATAATG |
| | | CCAAGACAAAGCCGCGGGAGGAGCAGTACAACAGCACGTACCGGGTGGTCAGCGTC |
| | | CTCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCCC |
| | | CGAGAACCACAGGTGTACACCCTGCCCCCATCCCGGGAGGAGATGACCAAGAACCA |
| | | GGTCAGCCTGACCTGCCTGGTCAAAGGCTTCTATCCCAGCGACATCGCCGTGGAGTG |
| | | GGAGAGCAATGGGCAGCCGGAGAACAACTACAAGACCACGCCTCCCGTGCTGGACT |
| | | CCGACGGCTCCTTCTTCCTCTACAGCAAGCTCACCGTGGACAAGAGCAGGTGGCAGC |
| | | AGGGGAACGTCTTCTCATGCTCCGTGATGCATGAGGCTCTGCACAACCACTACACGC |
| | | AGAAGAGCCTCTCCCTGTCTCCGGGTAAA |
| SEQ ID | DNA | CAGAGCGCCCTGACCCAGCCCGCCAGCGTGTCCGGCAGCCCAGGCCAGTCTATCAC |
| NO171 | Light | AATCAGCTGCACCGGCACCTCCAGCGACGTGGGCAGCTACAACTACGTGAACTGGTA |
| | Chain | TCAGCAGCACCCCGGCAAGGCCCCCAAGCTGATGATCTACGGCGTGAGCAAGAGGC |
| | | CCAGCGGCGTGTCCAACAGGTTCAGCGGCAGCAAGAGCGGCAACACCGCCAGCCTG |
| | | ACAATCAGTGGGCTGCAGGCTGAGGACGAGGCCGACTACTACTGCGGCACCTTTGC |
| | | CGGCGGATCATACTACGGCGTGTTCGGCGGAGGGACCAAGCTGACCGTGCTGGGCC |
| | | AGCCTAAGGCTGCCCCCAGCGTGACCCTGTTCCCCCCCAGCAGCGAGGAGCTGCAG |
| | | GCCAACAAGGCCACCCTGGTGTGCCTGATCAGCGACTTCTACCCAGGCGCCGTGAC |
| | | CGTGGCCTGGAAGGCCGACAGCAGCCCCGTGAAGGCCGGCGTGGAGACCACCACC |
| | | CCCAGCAAGCAGAGCAACAACAAGTACGCCGCCAGCAGCTACCTGAGCCTGACCCC |
| | | CGAGCAGTGGAAGAGCCACAGGTCCTACAGCTGCCAGGTGACCCACGAGGGCAGCA |
| | | CCGTGGAAAAGACCGTGGCCCCAACCGAGTGCAGC |
| SEQ ID | DNA | CAGAGCGCCCTGACCCAGCCCGCCAGCGTGTCCGGCAGCCCAGGCCAGTCTATCAC |
| NO172 | Light | AATCAGCTGCACCGGCACCTCCAGCGACGTGGGCAGCTACAACTACGTGAACTGGTA |
| | Chain | TCAGCAGCACCCCGGCAAGGCCCCCAAGCTGATGATCTACGGCGTGAGCAAGAGGC |
| | | CCAGCGGCGTGTCCAACAGGTTCAGCGGCAGCAAGAGCGGCAACACCGCCAGCCTG |
| | | ACAATCAGTGGGCTGCAGGCTGAGGACGAGGCCGACTACTACTGCGGCACCTTTGC |
| | | CGGCGGATCATACTACGGCGTGTTCGGCGGAGGGACCAAGCTGACCGTGCTGGGCC |
| | | AGCCTAAGGCTGCCCCCAGCGTGACCCTGTTCCCCCCCAGCAGCGAGGAGCTGCAG |
| | | GCCAACAAGGCCACCCTGGTGTGCCTGATCAGCGACTTCTACCCAGGCGCCGTGAC |
| | | CGTGGCCTGGAAGGCCGACAGCAGCCCCGTGAAGGCCGGCGTGGAGACCACCACC |
| | | CCCAGCAAGCAGAGCAACAACAAGTACGCCGCCAGCAGCTACCTGAGCCTGACCCC |
| | | CGAGCAGTGGAAGAGCCACAGGTCCTACAGCTGCCAGGTGACCCACGAGGGCAGCA |
| | | CCGTGGAAAAGACCGTGGCCCCAACCGAGTGCAGC |
| SEQ ID | DNA | CAGAGCGCACTGACCCAGCCAGCTTCAGTGAGCGGCTCACCAGGTCAGAGCATTAC |
| NO173 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGAGCGCACTGACCCAGCCAGCTTCAGTGAGCGGCTCACCAGGTCAGAGCATTAC |
| NO174 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGAGCGCACTGACCCAGCCAGCTTCAGTGAGCGGCTCACCAGGTCAGAGCATTAC |
| NO175 | Light | CATCTCGTGTACGGGTACTAGCAGCGATGTTGGTTCTTATAATTATGTGAATTGGTAC |
| | Chain | CAGCAGCATCCCGGGAAGGCGCCGAAACTTATGATTTATGGTGTTTCTAAGCGTCCC |
| | | TCAGGCGTGAGCAACCGTTTTAGCGGATCCAAAAGCGGCAACACCGCGAGCCTGAC |
| | | CATTAGCGGCCTGCAAGCGGAAGACGAAGCGGATTATTATTGCGGTACTTTTGCTGG |
| | | TGGTTCTTATTATGGTGTGTTTGGCGGCGGCACGAAGTTAACCGTCCTAGGTCAGCC |
| | | CAAGGCTGCCCCCTCGGTCACTCTGTTCCCGCCCTCCTCTGAGGAGCTTCAAGCCAA |
| | | CAAGGCCACACTGGTGTGTCTCATAAGTGACTTCTACCCGGGAGCCGTGACAGTGGC |
| | | CTGGAAGGCAGATAGCAGCCCCGTCAAGGCGGGAGTGGAGACCACCACACCCTCCA |
| | | AACAAAGCAACAACAAGTACGCGGCCAGCAGCTATCTGAGCCTGACGCCTGAGCAGT |
| | | GGAAGTCCCACAGAAGCTACAGCTGCCAGGTCACGCATGAAGGGAGCACCGTGGAG |
| | | AAGACAGTGGCCCCTACAGAATGTTCA |
| SEQ ID | DNA | CAGGTGCAGCTGGTGCAGAGCGGAGCTGAGGTGAAGAAGCCAGGCGCCAGCGTCAA |
| NO176 | Heavy | GGTGTCCTGCAAGGCCAGCGGCTACACCTTCACCAGCAGCTACATCAACTGGGTCCG |
| | Chain | CCAGGCTCCTGGGCAGGGACTGGAGTGGATGGGCACCATCAACCCCGTGTCCGGCA |
| | | GCACCAGCTACGCCCAGAAGTTCCAGGGCAGAGTCACCATGACCAGGGACACCAGC |
| | | ATCAGCACCGCCTACATGGAGCTGTCCAGGCTGAGAAGCGACGACACCGCCGTGTA |
| | | CTACTGCGCCAGGGGCGGCTGGTTCGACTACTGGGGCCAGGGCACCCTGGTGACCG |
| | | TGTCCTCAGCTAGCACCAAGGGCCCCAGCGTGTTCCCCCTGGCCCCCTGCAGCAGA |
| | | AGCACCAGCGAGAGCACAGCCGCCCTGGGCTGCCTGGTGAAGGACTACTTCCCCGA |
| | | GCCAGTGACCGTGTCCTGGAACAGCGGAGCCCTGACCAGCGGCGTGCACACCTTCC |
| | | CCGCCGTGCTGCAGAGCAGCGGCCTGTACAGCCTGTCCAGCGTGGTGACCGTGCCC |
| | | AGCAGCAACTTCGGCACCCAGACCTACACCTGCAACGTGGACCACAAGCCCAGCAAC |
| | | ACCAAGGTGGACAAGACCGTGGAGAGGAAGTGCTGCGTGGAGTGCCCCCCCTGCCC |
| | | AGCCCCCCCAGTGGCCGGACCCTCCGTGTTCCTGTTCCCCCCCAAGCCCAAGGACA |
| | | CCCTGATGATCAGCAGGACCCCCGAGGTGACCTGCGTGGTGGTGGACGTGAGCCAC |
| | | GAGGACCCAGAGGTGCAGTTCAACTGGTACGTGGACGGCGTGGAGGTGCACAACGC |
| | | CAAGACCAAGCCCAGAGAGGAACAGTTTAACAGCACCTTCAGGGTGGTGTCCGTGCT |
| | | GACCGTGGTGCACCAGGACTGGCTGAACGGCAAAGAGTACAAGTGCAAGGTCTCCA |
| | | ACAAGGGCCTGCCAGCCCCCATCGAGAAAACCATCAGCAAGACCAAGGGCCAGCCA |
| | | CGGGAGCCCCAGGTGTACACCCTGCCCCCCAGCCGGGAGGAAATGACCAAGAACCA |
| | | GGTGTCCCTGACCTGTCTGGTGAAGGGCTTCTACCCCAGCGACATCGCCGTGGAGT |
| | | GGGAGAGCAACGGCCAGCCCGAGAACAACTACAAGACCACCCCCCCCATGCTGGAC |
| | | AGCGACGGCAGCTTCTTCCTGTACAGCAAGCTGACAGTGGACAAGAGCAGGTGGCA |
| | | GCAGGGCAACGTGTTCAGCTGCAGCGTGATGCACGAGGCCCTGCACAACCACTACA |
| | | CCCAGAAGAGCCTGAGCCTGTCCCCCGGCAAG |
| SEQ ID | DNA | CAGGTGCAGCTGGTGCAGAGCGGAGCTGAGGTGAAGAAGCCAGGCGCCAGCGTCAA |
| NO177 | Heavy | GGTGTCCTGCAAGGCCAGCGGCTACACCTTCACCAGCAGCTACATCAACTGGGTGCG |
| | Chain | CCAGGCTCCAGGGCAGGGACTGGAGTGGATGGGCCAGATCAACGCCGCCAGCGGC |
| | | ATGACCAGATACGCCCAGAAGTTCCAGGGCAGAGTCACAATGACCAGGGACACCTCT |
| | | ATCAGCACCGCCTACATGGAGCTGTCCAGGCTGAGAAGCGACGACACCGCCGTGTA |
| | | CTACTGCGCCAGGGGCGGCTGGTTCGACTACTGGGGCCAGGGCACCCTGGTGACCG |
| | | TGTCCTCAGCTAGCACCAAGGGCCCCAGCGTGTTCCCCCTGGCCCCCTGCAGCAGA |
| | | AGCACCAGCGAGAGCACAGCCGCCCTGGGCTGCCTGGTGAAGGACTACTTCCCCGA |
| | | GCCAGTGACCGTGTCCTGGAACAGCGGAGCCCTGACCAGCGGCGTGCACACCTTCC |
| | | CCGCCGTGCTGCAGAGCAGCGGCCTGTACAGCCTGTCCAGCGTGGTGACCGTGCCC |
| | | AGCAGCAACTTCGGCACCCAGACCTACACCTGCAACGTGGACCACAAGCCCAGCAAC |
| | | ACCAAGGTGGACAAGACCGTGGAGAGGAAGTGCTGCGTGGAGTGCCCCCCCTGCCC |
| | | AGCCCCCCCAGTGGCCGGACCCTCCGTGTTCCTGTTCCCCCCCAAGCCCAAGGACA |
| | | CCCTGATGATCAGCAGGACCCCCGAGGTGACCTGCGTGGTGGTGGACGTGAGCCAC |
| | | GAGGACCCAGAGGTGCAGTTCAACTGGTACGTGGACGGCGTGGAGGTGCACAACGC |
| | | CAAGACCAAGCCCAGAGAGGAACAGTTTAACAGCACCTTCAGGGTGGTGTCCGTGCT |
| | | GACCGTGGTGCACCAGGACTGGCTGAACGGCAAAGAGTACAAGTGCAAGGTCTCCA |
| | | ACAAGGGCCTGCCAGCCCCCATCGAGAAAACCATCAGCAAGACCAAGGGCCAGCCA |
| | | CGGGAGCCCCAGGTGTACACCCTGCCCCCCAGCCGGGAGGAAATGACCAAGAACCA |
| | | GGTGTCCCTGACCTGTCTGGTGAAGGGCTTCTACCCCAGCGACATCGCCGTGGAGT |
| | | GGGAGAGCAACGGCCAGCCCGAGAACAACTACAAGACCACCCCCCCCATGCTGGAC |
| | | AGCGACGGCAGCTTCTTCCTGTACAGCAAGCTGACAGTGGACAAGAGCAGGTGGCA |
| | | GCAGGGCAACGTGTTCAGCTGCAGCGTGATGCACGAGGCCCTGCACAACCACTACA |
| | | CCCAGAAGAGCCTGAGCCTGTCCCCCGGCAAG |
| SEQ ID | DNA | CAGGTGCAATTGGTTCAGAGCGGCGCGGAAGTGAAAAAACCGGGCGCGAGCGTGAA |
| NO178 | Heavy | AGTGAGCTGCAAAGCCTCCGGATATACCTTTACTTCTTCTTATATTAATTGGGTCCGCC |
| | Chain | AAGCCCCTGGGCAGGGTCTCGAGTGGATGGGCAATATTAATGCTGCTGCTGGTATTA |
| | | CTCTTTATGCTCAGAAGTTTCAGGGTCGGGTCACCATGACCCGTGATACCAGCATTAG |
| | | CACCGCGTATATGGAACTGAGCCGCCTGCGTAGCGATGATACGGCCGTGTATTATTG |
| | | CGCGCGTGGTGGTTGGTTTGATTATTGGGGCCAAGGCACCCTGGTGACGGTTAGCTC |
| | | AGCTTCCACCAAGGGCCCCAGCGTGTTCCCCCTGGCCCCCTGCAGCAGAAGCACCA |
| | | GCGAGAGCACAGCCGCCCTGGGCTGCCTGGTGAAGGACTACTTCCCCGAGCCCGTG |
| | | ACCGTGAGCTGGAACAGCGGAGCCCTGACCAGCGGCGTGCACACCTTCCCCGCCGT |
| | | GCTGCAGAGCAGCGGCCTGTACAGCCTGAGCAGCGTGGTGACCGTGCCCAGCAGCA |
| | | ACTTCGGCACCCAGACCTACACCTGCAACGTGGACCACAAGCCCAGCAACACCAAGG |
| | | TGGACAAGACCGTGGAGCGGAAGTGCTGCGTGGAGTGCCCCCCCTGCCCTGCCCCT |
| | | CCTGTGGCCGGACCCTCCGTGTTCCTGTTCCCCCCCAAGCCCAAGGACACCCTGATG |
| | | ATCAGCCGGACCCCCGAGGTGACCTGCGTGGTGGTGGACGTGAGCCACGAGGACCC |
| | | CGAGGTGCAGTTCAACTGGTACGTGGACGGCGTGGAGGTGCACAACGCCAAGACCA |
| | | AGCCCCGGGAGGAACAGTTCAACAGCACCTTCCGGGTGGTGTCCGTGCTGACCGTG |
| | | GTGCACCAGGACTGGCTGAACGGCAAAGAATACAAGTGCAAGGTGTCCAACAAGGG |
| | | CCTGCCTGCCCCCATCGAGAAAACCATCAGCAAGACAAAGGGCCAGCCCAGGGAAC |
| | | CCCAGGTGTACACCCTGCCCCCCAGCCGGGAGGAAATGACCAAGAACCAGGTGTCC |
| | | CTGACCTGTCTGGTGAAGGGCTTCTACCCCAGCGACATCGCCGTGGAGTGGGAGAG |
| | | CAACGGCCAGCCCGAGAACAACTACAAGACCACCCCCCCCATGCTGGACAGCGACG |
| | | GCAGCTTCTTCCTGTACAGCAAGCTGACAGTGGACAAGAGCCGGTGGCAGCAGGGC |
| | | AACGTGTTCAGCTGCAGCGTGATGCACGAGGCCCTGCACAACCACTACACCCAGAAG |
| | | AGCCTGAGCCTGTCCCCCGGCAAA |
| SEQ ID | DNA | CAGGTGCAATTGGTTCAGAGCGGCGCGGAAGTGAAAAAACCGGGCGCGAGCGTGAA |
| NO179 | Heavy | AGTGAGCTGCAAAGCCTCCGGATATACCTTTACTTCTTCTTATATTAATTGGGTCCGCC |
| | Chain | AAGCCCCTGGGCAGGGTCTCGAGTGGATGGGCGGTATTAATCCTCCTGCTGGTACTA |
| | | CTTCTTATGCTCAGAAGTTTCAGGGTCGGGTCACCATGACCCGTGATACCAGCATTAG |
| | | CACCGCGTATATGGAACTGAGCCGCCTGCGTAGCGATGATACGGCCGTGTATTATTG |
| | | CGCGCGTGGTGGTTGGTTTGATTATTGGGGCCAAGGCACCCTGGTGACGGTTAGCTC |
| | | AGCTTCCACCAAGGGCCCCAGCGTGTTCCCCCTGGCCCCCTGCAGCAGAAGCACCA |
| | | GCGAGAGCACAGCCGCCCTGGGCTGCCTGGTGAAGGACTACTTCCCCGAGCCCGTG |
| | | ACCGTGAGCTGGAACAGCGGAGCCCTGACCAGCGGCGTGCACACCTTCCCCGCCGT |
| | | GCTGCAGAGCAGCGGCCTGTACAGCCTGAGCAGCGTGGTGACCGTGCCCAGCAGCA |
| | | ACTTCGGCACCCAGACCTACACCTGCAACGTGGACCACAAGCCCAGCAACACCAAGG |
| | | TGGACAAGACCGTGGAGCGGAAGTGCTGCGTGGAGTGCCCCCCCTGCCCTGCCCCT |
| | | CCTGTGGCCGGACCCTCCGTGTTCCTGTTCCCCCCCAAGCCCAAGGACACCCTGATG |
| | | ATCAGCCGGACCCCCGAGGTGACCTGCGTGGTGGTGGACGTGAGCCACGAGGACCC |
| | | CGAGGTGCAGTTCAACTGGTACGTGGACGGCGTGGAGGTGCACAACGCCAAGACCA |
| | | AGCCCCGGGAGGAACAGTTCAACAGCACCTTCCGGGTGGTGTCCGTGCTGACCGTG |
| | | GTGCACCAGGACTGGCTGAACGGCAAAGAATACAAGTGCAAGGTGTCCAACAAGGG |
| | | CCTGCCTGCCCCCATCGAGAAAACCATCAGCAAGACAAAGGGCCAGCCCAGGGAAC |
| | | CCCAGGTGTACACCCTGCCCCCCAGCCGGGAGGAAATGACCAAGAACCAGGTGTCC |
| | | CTGACCTGTCTGGTGAAGGGCTTCTACCCCAGCGACATCGCCGTGGAGTGGGAGAG |
| | | CAACGGCCAGCCCGAGAACAACTACAAGACCACCCCCCCCATGCTGGACAGCGACG |
| | | GCAGCTTCTTCCTGTACAGCAAGCTGACAGTGGACAAGAGCCGGTGGCAGCAGGGC |
| SEQ ID | Antibody | GCWLDDFNC |
| NO186 | binding | |
| region | | |
| SEQ ID | Antibody | CEGEQDKRLHCYASW |
| NO187 | binding | |
| region | | |
| SEQ ID | Antibody | WLDDFN |
| NO188 | binding | |
| region | | |
| SEQ ID | Antibody | EQDKR |
| NO189 | binding | |
| region | | |
| SEQ ID | Antibody | KGCWLDDFNCY |
| NO190 | binding | |
| region | | |
| SEQ ID | Antibody | CIYYNANWELERT |
| NO191 | binding | |
| region | | |
| SEQ ID | Antibody | YFCCCEGNFCN |
| NO192 | binding | |
| region | | |
| SEQ ID | Light- | DIALTQPASVSGSPGQSITISCTGTSSDVGSYNYVNWYQQHPGKAPKLMIYGVSKRPSGV |
| NO193 | h/mIgG2 | SNRFSGSKSGNTASLTISGLQAEDEADYYCGTFAGGSYYGVFGGGTKLTVLGQPKSTPTL |
| | aLALA | TVFPPSSEELKENKATLVCLISNFSPSGVTVAWKANGTPITQGVDTSNPTKEGNKFMASS |
| | Chain | FLHLTSDQWRSHNSFTCQVTHEGDTVEKSLSPAECL |
| SEQ ID | Heavy- | QVQLVQSGAEVKKPGASVKVSCKASGYTFTSSYINWVRQAPGQGLEWMGTINPVSGST |
| NO194 | h/mIgG2 | SYAQKFQGRVTMTRDTSISTAYMELSSLRSEDTAVYYCARGGWFDYWGQGTLVTVSSA |
| | aLALA | KTTAPSVYPLAPVCGDTTGSSVTLGCLVKGYFPEPVTLTWNSGSLSSGVHTFPAVLQSDL |
| | chain | YTLSSSVTVTSSTWPSQSITCNVAHPASSTKVDKKIEPRGPTIKPCPPCKCPAPNAAGGPS |
| | | VFIFPPKIKDVLMISLSPTVTCVVVDVSEDDPDVQISWFVNNVEVHTAQTQTHREDYNSTLR |
| | | VVSALPIQHQDWMSGKEFKCKVNNKDLPAPIERTISKPKGSVRAPQVYVLPPPEEEMTKK |
| | | QVTLTCMVTDFMPEDIYVEWTNNGKTELNYKNTEPVLDSDGSYFMYSKLRVEKKNWVER |
| | | NSYSCSVVHEGLHNHHTTKSFSRTPGK |

The embodiments of the disclosed methods, treatments, regimens, uses and kits employ an ActRII receptor antagonist, e.g., an ActRIIB binding molecule. In further embodiments, the ActRIIB binding molecule is an antagonistic antibody to ActRIIB.

In some embodiments of the disclosed methods, treatments, regimens, uses and kits, the antibody is bimagrumab.

The details of one or more embodiments of the disclosure are set forth in the accompanying description above. Any methods and materials similar or equivalent to those described herein can be used in the practice or testing of the present disclosure. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. The following examples are meant to more fully illustrate the disclosure and are not meant in any way to limit the scope thereof.

### EXAMPLES

### General Methodology

ActRIIB antibodies, their characterization and methods related thereto like (i) Functional Assays, (ii) REPORTER GENE ASSAYs (RGA), (iii) Cultivation of HEK293T/17 Cell Lines, (iv) Myostatin-Induced Luciferase Reporter Gene Assays, (v) SPECIFICITY ELISAs, (vi) ActRIIB/Fc-Myostatin Binding Interaction ELISA, (vii) FACS titration on hActRIIB- and hActRIIA-Expressing Cells, (viii) Binding to primary human skeletal muscle cells, (ix) affinity Determination of Selected Anti-Human ActRIIB Fabs Using Surface Plasmon Resonance (Biacore), (x) CK ASSAY, (xi) Animal Models, (xii) TREATMENT PROTOCOLs, (xiii) Statistical Analysis, (xiiii) Pannings, (xv) antibody identification and characterization, (xvi) Optimization of antibodies derived from first affinity maturation, (xvii) IgG2 Conversion of Affinity Matured Fabs (1st Maturation), (xviiii) Second Affinity Maturation, (xx) IgG2 Conversion and Characterization of IgG2 (2nd Maturation), (xxi) Characterization of anti-ActRIIB antibodies in in vivo murine studies, (xxii) Confirmation of affinity by SET, (xxiii) Cross Blocking Studies and (xxiv) Epitope mapping details and technologies have been disclosed in the WO 2010/125003.

To study whether the ActRII receptor antagonist Bimagrumab can be used for developing a treatment for stress urinary incontinence, a dual injury childbirth simulation rat model is used. Said dual injury childbirth simulation rat model has been disclosed in Hai-Hong Jiang et al., Dual simulated childbirth injuries result in slowed recovery of pudendal nerve and urethral function; Neurourol Urodyn. 2009; 28(3): 229-235 and Song et al., Combination Histamine and Serotonin Treatment After Simulated Childbirth Injury Improves Stress Urinary; Neurourology and Urodynamics 35:703-710 (2016)). The Material and Method sections entitled Animal preparations, Childbirth simulation injury models and Leak point pressure (LPP) with simultaneous neuromuscular physiological recordings of Jiang et al., 2009 are incorporated by reference herein as if fully set forth.

The rat stress urinary incontinence model described by Hai-Hong Jiang et al., 2009 and Song et al, 2016, induced by pudendal nerve crush and vaginal distension in female, virgin Sprague Dawley rats (200-250 g), is used to study the effect of bimagrumab on stress urinary incontinence. Bimagrumab administered in a therapeutic intervention modality, on leak point pressure (LPP) and external urethral sphincter (EUS) electromyography (EMG) in the above described experimental rat model of stress urinary incontinence, induced by pudendal nerve crush and vaginal distension (PNC+ VD) in female, virgin Sprague Dawley rats (200-250 g), can have a beneficial effect on stress urinary incontinence.

To investigate the effect of bimagrumab on stress urinary incontinence, the rats are treated according to the protocol described in Hai-Hong Jiang et al., 2009 one week after surgery.

TABLE 4
treatment regimen

| Group | Condition | Treatment | Dose (mg/kg) |
|---|---|---|---|
| A | Sham PNC + VD¹ | vehicle | 0 |
| B | PNC + VD | vehicle | 0 |
| C | PNC + VD | bimagrumab and vehicle | 10 |
| D | PNC + VD | clenbuterol | 0.1 |

¹Rat model of stress urinary incontinence, induced by pudendal nerve crush and vaginal distension (PNC + VD) in female, virgin Sprague Dawley rats (200-250 g); n = 8~10 (total 32~40);.

### Functional Readout:

In order to detect any potential statistically significant improvement upon interventions on LPP and/or EUSEMG compared to PNC+VD vehicle group and explore differences between intervention with bimagrumab and clenbuterol on stress urinary incontinence the following functional read-outs are assessed:
1. Response to leak point pressure (LPP) testing using pudendal nerve motor branch potential (PNMBP) recorded to assess nerve injury and neuroregeneration, and/or
2. Recording of external urethral sphincter (EUS) electromyography (EMG) to assess muscle injury and re-innervation, with the possibility to record leak point pressure (LPP) test with simultaneous external urethral sphincter electromyogram (EUS EMG) and pudendal nerve motor branch potential (PNMBP) recordings.
3. Body weight monitoring, hind limb skeletal muscle weight (e.g. quadriceps, gastrocnemius complex, tibialis anterior);

### Clinical Trials Using Placebo and the Acetate Salt Form of the Compound (R)-7-(2-(1-(4-butoxyphenyl)-2-methylpropan-2-ylamino)-1-hydroxyethyl)-5-hydroxybenzo[d]thiazol-2(3H)-one in Urinary Stress Incontinence

A clinical trial using the acetate salt form of the compound (R)-7-(2-(1-(4-butoxyphenyl)-2-methylpropan-2-ylamino)-1-hydroxyethyl)-5-hydroxybenzo[d]thiazol-2(3H)-one can be designed a described in the publication by Yasuda et al., A Double-Blind Clinical Trial of a/32-adrenergic Agonist in Stress Incontinence, Int Urogynecol J (1993) 4:146-151.

### Patient Selection:

Patients complaining about stress incontinence as well as patients having both stress and urge incontinence are selected. Furthermore, patients with a PPBC scale of 4 and 5 are selected. Urodynamic studies are performed in accordance with the rules of the International Continence society.

### Urodynamic Studies:

To assess the effect of the treatment the following studies are performed/data collected:
Urethal Pressure profile (e.g. in accordance with Brown and Wickham, J E A. The urethral pressure profile. Br J Urol 1969; 41:211-217)
Pad-Weighing Test (Joergense L. et al., One-hour pad weighing test for objective assessment of female urinary incontinence. Obstet Gynecol 1987; 69:39-42)
Daily frequency of incontinence/pad changing frequency: patients are directed to record incontinence episodes on a scale before, during and at the end of the treatment.
number of incontinence episodes per 24 hours;
number of micturitions per 24 hours;
volume voided per micturition/incontinence episode;
number of urgency incontinence episodes;
number of nocturia episodes per 24 hours;
number of involuntary leakage of urine accompanied by or immediately proceeded by urgency;
PPBC scale assessment

### Study End Points:

Primary end point: Change in frequency of daily stress urinary incontinence episodes from baseline at the beginning the study to end of study (e.g. 12 weeks).

### Secondary End Points:

1. Change in number of incontinence episodes per 24 hours;
2. Change in number of micturitions per 24 hours;
3. Change in volume voided per micturition/incontinence episode;
4. Change in number of urgency incontinence episodes;
5. Change in number of nocturia episodes per 24 hours;
6. Change in number of involuntary leakage of urine accompanied by or immediately proceeded by urgency;

The improvement in the severity of incontinence on the basis of the primary and secondary end point results are assessed by comparing the initial status and the posttreatment status.

Further preferred embodiments:

1. An ActRII receptor antagonist for use in treating a subject showing symptoms of urinary incontinence or is at risk of developing urinary incontinence.
2. An ActRII receptor antagonist for use in treating urinary incontinence according to embodiment 1, wherein the urinary incontinence is caused by, or associated with a pelvic floor disorders resulting from a weakened or damaged pelvic muscle.
3. An ActRII receptor antagonist for use in treating urinary incontinence according to embodiment 1 or 2, wherein said urinary incontinence is an incontinence selected from the group consisting of stress urinary incontinence, urge urinary incontinence and reflex urinary incontinence.
4. An ActRII receptor antagonist for use in treating urinary incontinence according to embodiment 3, wherein said urinary incontinence is stress urinary incontinence.
5. An ActRII receptor antagonist for use in treating urinary incontinence according to embodiment 2 wherein said weakened or damaged pelvic muscle is the musculus levator ani, musculus bulbocavernosus or musculus sphincter urethrae externus.
6. An ActRII receptor antagonist for use in treating urinary incontinence according to embodiments 1-5, wherein said urinary incontinence is related to or caused by the effects of childbirth or menopause.
7. A method for treating urinary incontinence, said method comprising administering an effective amount of an ActRII receptor antagonist to a subject who shows symptoms of urinary incontinence or is at risk for developing urinary incontinence.
8. The method of embodiment 7, wherein the urinary incontinence is caused by, or associated with, a pelvic floor disorders resulting from a weakened or damaged pelvic muscle.
9. The method according to embodiment 8, wherein said urinary incontinence is an incontinence selected from the group consisting of stress urinary incontinence, urge urinary incontinence and reflex urinary incontinence.
10. The method according to embodiment 9, wherein said weakened or damaged pelvic muscle is the musculus levator ani, musculus bulbocavernosus or musculus sphincter urethrae externus.
11. The method according to embodiment 10, wherein the urinary incontinence is related to or caused by the effects of childbirth or menopause.
12. A method of treating a pelvic muscle abnormality associated with an urinary incontinence condition selected from the group consisting of: stress urinary incontinence, urge urinary incontinence and reflex urinary incontinence, said method comprising administering an effective amount of an ActRII receptor antagonist to a subject having said pelvic muscle functional abnormality.
13. An ActRII receptor antagonist for use according to any one of embodiments 1-6 or a method of treatment according to any one of embodiments 7-12, wherein the ActRII receptor antagonist is an ActRII receptor binding molecule.
14. An ActRII receptor antagonist for use according to any one of embodiments 1-6 or a method of treatment according to any one of embodiments 7-12, wherein the ActRII receptor antagonist binds to the ActRIIA and/or to the ActRIIB receptor.
15. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof.
16. An ActRII receptor antagonist for use according to any one of embodiments 1-6 or a method of treatment according to any one of embodiments 7-12, wherein the anti-ActRII receptor antibody is bimagrumab or an antigen-binding portion thereof.
17. An ActRII receptor antagonist for use according to any one of embodiments 1-6 or a method of treatment according to any one of embodiments 7-12, wherein the ActRII receptor antagonist is an anti-ActRII antibody or an antigen-binding portion thereof that binds to an epitope of ActRIIB consisting of amino acids 19-134 of SEQ ID NO: 181 (SEQ ID NO: 182).
18. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the anti-ActRII antibody or an antigen-binding portion thereof binds to an epitope of ActRIIB comprising or consisting of:

(a) amino acids 78-83 of SEQ ID NO: 181
(WLDDFN-SEQ ID NO: 188);
(b) amino acids 76-84 of SEQ ID NO: 181
(GCWLDDFNC-SEQ ID NO: 186);
(c) amino acids 75-85 of SEQ ID NO: 181
(KGCWLDDFNCY-SEQ ID NO: 190);
(d) amino acids 52-56 of SEQ ID NO: 181
(EQDKR-SEQ ID NO: 189);
(e) amino acids 49-63 of SEQ ID NO: 181
(CEGEQDKRLHCYASW-SEQ ID NO: 187);
(f) amino acids 29-41 of SEQ ID NO: 181
(CIYYNANWELERT-SEQ ID NO: 191);
(g) amino acids 100-110 of SEQ ID NO: 181
(YFCCCEGNFCN-SEQ ID NO: 192);
or
(h) amino acids 78-83 of SEQ ID NO: 181
(WLDDFN)
and
amino acids 52-56 of SEQ ID NO: 181
(EQDKR).

19. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the anti-ActRII receptor antibody or an antigen-binding portion thereof is selected from the group consisting of:
a) an anti-ActRIIB antibody or antigen binding portion thereof that binds to an epitope of ActRIIB comprising:

i. amino acids 78-83 of SEQ ID NO: 181
(WLDDFN-SEQ ID NO: 188);
ii. amino acids 76-84 of SEQ ID NO: 181
(GCWLDDFNC-SEQ ID NO: 186);
iii. amino acids 75-85 of SEQ ID NO: 181
(KGCWLDDFNCY-SEQ ID NO: 190);
iv. amino acids 52-56 of SEQ ID NO: 181
(EQDKR-SEQ ID NO: 189);
v. amino acids 49-63 of SEQ ID NO: 181
(CEGEQDKRLHCYASW-SEQ ID NO: 187);
vi. amino acids 29-41 of SEQ ID NO: 181
(CIYYNANWELERT-SEQ ID NO: 191);
vii. amino acids 100-110 of SEQ ID NO: 181
(YFCCCEGNFCN-SEQ ID NO: 192);
or
viii. amino acids 78-83 of SEQ ID NO: 181
(WLDDFN)
and
amino acids 52-56 of SEQ ID NO: 181
(EQDKR);
and

b) an antagonist antibody to ActRIIB that binds to an epitope of ActRIIB comprising:

i. amino acids 78-83 of SEQ ID NO: 181
(WLDDFN-SEQ ID NO: 188);
ii. amino acids 76-84 of SEQ ID NO: 181
(GCWLDDFNC-SEQ ID NO: 186);
iii. amino acids 75-85 of SEQ ID NO: 181
(KGCWLDDFNCY-SEQ ID NO: 190);
iv. amino acids 52-56 of SEQ ID NO: 181
(EQDKR-SEQ ID NO: 189);
v. amino acids 49-63 of SEQ ID NO: 181
(CEGEQDKRLHCYASW-SEQ ID NO: 187);
vi. amino acids 29-41 of SEQ ID NO: 181
(CIYYNANWELERT-SEQ ID NO: 191);
vii. amino acids 100-110 of SEQ ID NO: 181
(YFCCCEGNFCN-SEQ ID NO: 192);
or
viii. amino acids 78-83 of SEQ ID NO: 181
(WLDDFN)
and
amino acids 52-56 of SEQ ID NO: 181
(EQDKR), wherein the antibody has a KID of about
2 pM.

20. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody or an antigen-binding portion thereof binds to human ActRIIB with a 10-fold or greater affinity than it binds to human ActRIIA.
21. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody or an antigen-binding portion thereof comprises a heavy chain variable region CDR1 comprising an amino acid sequence selected from the group consisting of SEQ ID NOs: 1-14; a heavy chain variable region CDR2 comprising an amino acid sequence selected from the group consisting of SEQ ID NOs: 15-28; a heavy chain variable region CDR3 comprising an amino acid sequence selected from the group consisting of SEQ ID NOs: 29-42; a light chain variable region CDR1 comprising an amino acid sequence selected from the group consisting of SEQ ID NOs: 43-56; a light chain variable region CDR2 comprising an amino acid sequence selected from the group consisting of SEQ ID NOs: 57-70; and a light chain variable region CDR3 comprising an amino acid sequence selected from the group consisting of SEQ ID NOs: 71-84.
22. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody or an antigen-binding portion thereof comprises:
(a) a heavy chain variable region CDR1 of SEQ ID NO: 1; a heavy chain variable region CDR2 of SEQ ID NO: 15; a heavy chain variable region CDR3 of SEQ ID NO: 29; a light chain variable region CDR1 of SEQ ID NO: 43; a light chain variable region CDR2 of SEQ ID NO: 57; and a light chain variable region CDR3 of SEQ ID NO: 71,
(b) a heavy chain variable region CDR1 of SEQ ID NO: 2; a heavy chain variable region CDR2 of SEQ ID NO: 16; a heavy chain variable region CDR3 of SEQ ID NO: 30; a light chain variable region CDR1 of SEQ ID NO: 44; a light chain variable region CDR2 of SEQ ID NO: 58; and a light chain variable region CDR3 of SEQ ID NO: 72,
(c) a heavy chain variable region CDR1 of SEQ ID NO: 3; a heavy chain variable region CDR2 of SEQ ID NO: 17; a heavy chain variable region CDR3 of SEQ ID NO: 31; a light chain variable region CDR1 of SEQ ID NO: 45; a light chain variable region CDR2 of SEQ ID NO: 59; and a light chain variable region CDR3 of SEQ ID NO: 73,
(d) a heavy chain variable region CDR1 of SEQ ID NO: 4; a heavy chain variable region CDR2 of SEQ ID NO: 18; a heavy chain variable region CDR3 of SEQ ID NO: 32; a light chain variable region CDR1 of SEQ ID NO: 46; a light chain variable region CDR2 of SEQ ID NO: 60; and a light chain variable region CDR3 of SEQ ID NO: 74,
(e) a heavy chain variable region CDR1 of SEQ ID NO: 5; a heavy chain variable region CDR2 of SEQ ID NO: 19; a heavy chain variable region CDR3 of SEQ ID NO: 33; a light chain variable region CDR1 of SEQ ID NO: 47; a light chain variable region CDR2 of SEQ ID NO: 61; and a light chain variable region CDR3 of SEQ ID NO: 75,
(f) a heavy chain variable region CDR1 of SEQ ID NO: 6; a heavy chain variable region CDR2 of SEQ ID NO: 20; a heavy chain variable region CDR3 of SEQ ID NO: 34; a light chain variable region CDR1 of SEQ ID NO: 48; a light chain variable region CDR2 of SEQ ID NO: 62; and a light chain variable region CDR3 of SEQ ID NO: 76,
(g) a heavy chain variable region CDR1 of SEQ ID NO: 7; a heavy chain variable region CDR2 of SEQ ID NO: 21; a heavy chain variable region CDR3 of SEQ ID NO: 35; a light chain variable region CDR1 of SEQ ID NO: 49; a light chain variable region CDR2 of SEQ ID NO: 63; and a light chain variable region CDR3 of SEQ ID NO: 77,
(h) a heavy chain variable region CDR1 of SEQ ID NO: 8; a heavy chain variable region CDR2 of SEQ ID NO: 22; a heavy chain variable region CDR3 of SEQ ID NO: 36; a light chain variable region CDR1 of SEQ ID NO: 50 a light chain variable region CDR2 of SEQ ID NO: 64; and a light chain variable region CDR3 of SEQ ID NO: 78,
(i) a heavy chain variable region CDR1 of SEQ ID NO: 9; a heavy chain variable region CDR2 of SEQ ID NO: 23; a heavy chain variable region CDR3 of SEQ ID NO: 37; a light chain variable region CDR1 of SEQ ID NO: 51; a light chain variable region CDR2 of SEQ ID NO: 65; and a light chain variable region CDR3 of SEQ ID NO: 79,
(j) a heavy chain variable region CDR1 of SEQ ID NO: 10; a heavy chain variable region CDR2 of SEQ ID NO: 24; a heavy chain variable region CDR3 of SEQ ID NO: 38; a light chain variable region CDR1 of SEQ ID NO: 52; a light chain variable region CDR2 of SEQ ID NO: 66; and a light chain variable region CDR3 of SEQ ID NO: 80,
(k) a heavy chain variable region CDR1 of SEQ ID NO: 11; a heavy chain variable region CDR2 of SEQ ID NO: 25; a heavy chain variable region CDR3 of SEQ ID NO: 39; a light chain variable region CDR1 of SEQ ID NO: 53; a light chain variable region CDR2 of SEQ ID NO: 67; and a light chain variable region CDR3 of SEQ ID NO: 81,
(l) a heavy chain variable region CDR1 of SEQ ID NO: 12; a heavy chain variable region CDR2 of SEQ ID NO: 26; a heavy chain variable region CDR3 of SEQ ID NO: 40; a light chain variable region CDR1 of SEQ ID NO: 54; a light chain variable region CDR2 of SEQ ID NO: 68; and a light chain variable region CDR3 of SEQ ID NO: 82,
(m) a heavy chain variable region CDR1 of SEQ ID NO: 13; a heavy chain variable region CDR2 of SEQ ID NO: 27; a heavy chain variable region CDR3 of SEQ ID NO: 41; a light chain variable region CDR1 of SEQ ID NO: 55; a light chain variable region CDR2 of SEQ ID NO: 69; and a light chain variable region CDR3 of SEQ ID NO: 83, or
(n) a heavy chain variable region CDR1 of SEQ ID NO: 14; a heavy chain variable region CDR2 of SEQ ID NO: 28; a heavy chain variable region CDR3 of SEQ ID NO: 42; a light chain variable region CDR1 of SEQ ID NO: 56; a light chain variable region CDR2 of SEQ ID NO: 70; and a light chain variable region CDR3 of SEQ ID NO: 84.
23. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody comprises a full-length heavy chain amino acid sequence having at least 95% sequence identity to at least one sequence selected from the group consisting of SEQ ID NOs: 146-150 and 156-160 and a full-length light chain amino acid sequence having at least 95% sequence identity to at least one sequence selected from the group consisting of SEQ ID NOs: 141-145 and 151-155.
24. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody or an antigen-binding portion thereof comprises:
(a) the variable heavy chain sequence of SEQ ID NO: 99 and variable light chain sequence of SEQ ID NO: 85;
(b) the variable heavy chain sequence of SEQ ID NO: 100 and variable light chain sequence of SEQ ID NO: 86;
(c) the variable heavy chain sequence of SEQ ID NO: 101 and variable light chain sequence of SEQ ID NO: 87;
(d) the variable heavy chain sequence of SEQ ID NO: 102 and variable light chain sequence of SEQ ID NO: 88;
(e) the variable heavy chain sequence of SEQ ID NO: 103 and variable light chain sequence of SEQ ID NO: 89;
(f) the variable heavy chain sequence of SEQ ID NO: 104 and variable light chain sequence of SEQ ID NO: 90;
(g) the variable heavy chain sequence of SEQ ID NO: 105 and variable light chain sequence of SEQ ID NO: 91;
(h) the variable heavy chain sequence of SEQ ID NO: 106 and variable light chain sequence of SEQ ID NO: 92;
(i) the variable heavy chain sequence of SEQ ID NO: 107 and variable light chain sequence of SEQ ID NO: 93;
(j) the variable heavy chain sequence of SEQ ID NO: 108 and variable light chain sequence of SEQ ID NO: 94;
(k) the variable heavy chain sequence of SEQ ID NO: 109 and variable light chain sequence of SEQ ID NO: 95;
(l) the variable heavy chain sequence of SEQ ID NO: 110 and variable light chain sequence of SEQ ID NO: 96;
(m) the variable heavy chain sequence of SEQ ID NO: 111 and variable light chain sequence of SEQ ID NO: 97; or
(n) the variable heavy chain sequence of SEQ ID NO: 112 and variable light chain sequence of SEQ ID NO: 98.
25. An ActRII receptor antagonist for use or a method according to any one of embodiments 15-24, wherein the antibody comprises:
(a) the heavy chain sequence of SEQ ID NO: 146 and light chain sequence of SEQ ID NO: 141;
(b) the heavy chain sequence of SEQ ID NO: 147 and light chain sequence of SEQ ID NO: 142;
(c) the heavy chain sequence of SEQ ID NO: 148 and light chain sequence of SEQ ID NO: 143;
(d) the heavy chain sequence of SEQ ID NO: 149 and light chain sequence of SEQ ID NO: 144;
(e) the heavy chain sequence of SEQ ID NO: 150 and light chain sequence of SEQ ID NO: 145;
(f) the heavy chain sequence of SEQ ID NO: 156 and light chain sequence of SEQ ID NO: 151;
(g) the heavy chain sequence of SEQ ID NO: 157 and light chain sequence of SEQ ID NO: 152;
(h) the heavy chain sequence of SEQ ID NO: 158 and light chain sequence of SEQ ID NO: 153;
(i) the heavy chain sequence of SEQ ID NO: 159 and light chain sequence of SEQ ID NO: 154; or
(j) the heavy chain sequence of SEQ ID NO: 160 and light chain sequence of SEQ ID NO: 155.
26. An ActRII receptor antagonist for use according to any one of embodiments 1-6 or a method of treatment according to any one of embodiments 7-12, which is an anti-ActRII receptor antibody, wherein said antibody cross-blocks or is cross blocked by at least one antibody of embodiment 25 from binding to ActRIIB.
27. An ActRII receptor antagonist for use according to any one of embodiments 1-6 or a method of treatment according to any one of embodiments 7-12, which is an anti-ActRII receptor antibody, wherein the antibody has altered effector function through mutation of the Fc region.
28. An ActRII receptor antagonist for use according to any one of embodiments 1-6 or a method of treatment according to any one of embodiments 7-12, wherein the ActRII receptor antagonist is an anti-ActRII receptor antibody or an antigen-binding portion thereof, and wherein the antibody is encoded by pBW522 (DSM22873) or pBW524 (DSM22874).
29. Bimagrumab or an antigen-binding portion thereof for use in treating and/or preventing urinary incontinence.
30. Bimagrumab or an antigen-binding portion thereof for use in treating and/or preventing urinary incontinence according to embodiment 29, wherein said urinary incontinence is stress urinary incontinence, urge urinary incontinence and reflex urinary incontinence
31. Bimagrumab or an antigen-binding portion thereof for use in treating and/or preventing urinary incontinence according to embodiment 30, wherein said urinary incontinence is caused by, pelvic floor disorders resulting from a weakened or damaged pelvic muscle.
32. Bimagrumab or an antigen-binding portion thereof for use in treating and/or preventing urinary incontinence according to embodiment 31, wherein said weakened or damaged pelvic muscle is the musculus levator ani, musculus bulbocavernosus or musculus sphincter urethrae externus.
33. Bimagrumab or an antigen-binding portion thereof for use in treating and/or preventing urinary incontinence according to embodiment 32, wherein said weakened or damaged pelvic muscle is related to or caused by the effects of childbirth or the menopause.
34. A method for treating and/or preventing urinary incontinence, said method comprising administering an effective amount of bimagrumab to a subject who shows symptoms of urinary incontinence or is at risk for developing urinary incontinence.
35. The method according to embodiment 34, wherein said urinary incontinence is an incontinence selected from the group consisting of stress urinary incontinence, urge urinary incontinence and reflex urinary incontinence.
36. The method of embodiment 35, wherein the urinary incontinence is caused by, or associated with, a pelvic floor disorders resulting from a weakened or damaged pelvic muscle.
37. The method according to embodiment 36, wherein said weakened or damaged pelvic muscle is the musculus levator ani, musculus bulbocavernosus or musculus sphincter urethrae externus.
38. The method according to embodiment 37, wherein the urinary incontinence is related to or caused by the effects of childbirth or the menopause.
39. A method of treating a pelvic muscle abnormality associated with a urinary incontinence condition selected from the group consisting of: stress urinary incontinence, urge urinary incontinence and reflex urinary incontinence, said method comprising administering an effective amount of bimagrumab to a subject having said pelvic muscle functional abnormality.

What is claimed is:

1. A method for treating urinary incontinence, comprising administering an effective amount of an ActRII receptor antagonist to a subject having symptoms of urinary incontinence or at risk for developing urinary incontinence.

2. The method according to claim 1, wherein said urinary incontinence is selected from the group consisting of stress urinary incontinence, urge urinary incontinence and reflex urinary incontinence.

3. The method according to claim 1, wherein the ActRII receptor antagonist is an ActRII receptor binding molecule.

4. The method according to claim 1, wherein the ActRII receptor antagonist is an anti-ActRII receptor antibody or an antigen-binding portion thereof.

5. The method according to claim 1, wherein the ActRII receptor antagonist is an anti-ActRII antibody or an antigen-binding portion thereof that binds to an epitope of ActRIIB consisting of amino acids 19-134 of SEQ ID NO: 181 (SEQ ID NO: 182).

6. The method according to claim 1, wherein the ActRII receptor antagonist is an anti-ActRII receptor antibody or an antigen-binding portion thereof, and wherein the antibody or an antigen-binding portion thereof comprises a heavy chain variable region CDR1 comprising an amino acid sequence selected from the group consisting of SEQ ID NOs: 1-14; a heavy chain variable region CDR2 comprising an amino acid sequence selected from the group consisting of SEQ ID NOs: 15-28; a heavy chain variable region CDR3 comprising an amino acid sequence selected from the group consisting of SEQ ID NOs: 29-42; a light chain variable region CDR1 comprising an amino acid sequence selected from the group consisting of SEQ ID NOs: 43-56; a light chain variable region CDR2 comprising an amino acid sequence selected from the group consisting of SEQ ID NOs: 57-70; and a light chain variable region CDR3 comprising an amino acid sequence selected from the group consisting of SEQ ID NOs: 71-84.

7. The method according to claim 1, wherein the ActRII receptor antagonist is an anti-ActRII receptor antibody or an antigen-binding portion thereof, and wherein the antibody or an antigen-binding portion thereof comprises:
(a) a heavy chain variable region CDR1 of SEQ ID NO: 1; a heavy chain variable region CDR2 of SEQ ID NO: 15; a heavy chain variable region CDR3 of SEQ ID NO: 29; a light chain variable region CDR1 of SEQ ID NO: 43; a light chain variable region CDR2 of SEQ ID NO: 57; and a light chain variable region CDR3 of SEQ ID NO: 71,
(b) a heavy chain variable region CDR1 of SEQ ID NO: 2; a heavy chain variable region CDR2 of SEQ ID NO: 16; a heavy chain variable region CDR3 of SEQ ID NO: 30; a light chain variable region CDR1 of SEQ ID NO: 44; a light chain variable region CDR2 of SEQ ID NO: 58; and a light chain variable region CDR3 of SEQ ID NO: 72,
(c) a heavy chain variable region CDR1 of SEQ ID NO: 3; a heavy chain variable region CDR2 of SEQ ID NO: 17; a heavy chain variable region CDR3 of SEQ ID NO: 31; a light chain variable region CDR1 of SEQ ID NO: 45; a light chain variable region CDR2 of SEQ ID NO: 59; and a light chain variable region CDR3 of SEQ ID NO: 73,
(d) a heavy chain variable region CDR1 of SEQ ID NO: 4; a heavy chain variable region CDR2 of SEQ ID NO: 18; a heavy chain variable region CDR3 of SEQ ID NO: 32; a light chain variable region CDR1 of SEQ ID NO: 46; a light chain variable region CDR2 of SEQ ID NO: 60; and a light chain variable region CDR3 of SEQ ID NO: 74,
(e) a heavy chain variable region CDR1 of SEQ ID NO: 5; a heavy chain variable region CDR2 of SEQ ID NO: 19; a heavy chain variable region CDR3 of SEQ ID NO: 33; a light chain variable region CDR1 of SEQ ID NO: 47; a light chain variable region CDR2 of SEQ ID NO: 61; and a light chain variable region CDR3 of SEQ ID NO: 75,
(f) a heavy chain variable region CDR1 of SEQ ID NO: 6; a heavy chain variable region CDR2 of SEQ ID NO: 20; a heavy chain variable region CDR3 of SEQ ID NO: 34; a light chain variable region CDR1 of SEQ ID NO: 48; a light chain variable region CDR2 of SEQ ID NO: 62; and a light chain variable region CDR3 of SEQ ID NO: 76,
(g) a heavy chain variable region CDR1 of SEQ ID NO: 7; a heavy chain variable region CDR2 of SEQ ID NO: 21; a heavy chain variable region CDR3 of SEQ ID NO: 35; a light chain variable region CDR1 of SEQ ID NO: 49; a light chain variable region CDR2 of SEQ ID NO: 63; and a light chain variable region CDR3 of SEQ ID NO: 77,
(h) a heavy chain variable region CDR1 of SEQ ID NO: 8; a heavy chain variable region CDR2 of SEQ ID NO: 22; a heavy chain variable region CDR3 of SEQ ID NO: 36; a light chain variable region CDR1 of SEQ ID NO: 50 a light chain variable region CDR2 of SEQ ID NO: 64; and a light chain variable region CDR3 of SEQ ID NO: 78,
(i) a heavy chain variable region CDR1 of SEQ ID NO: 9; a heavy chain variable region CDR2 of SEQ ID NO: 23; a heavy chain variable region CDR3 of SEQ ID NO: 37; a light chain variable region CDR1 of SEQ ID NO: 51; a light chain variable region CDR2 of SEQ ID NO: 65; and a light chain variable region CDR3 of SEQ ID NO: 79,
(j) a heavy chain variable region CDR1 of SEQ ID NO: 10; a heavy chain variable region CDR2 of SEQ ID NO: 24; a heavy chain variable region CDR3 of SEQ ID NO: 38; a light chain variable region CDR1 of SEQ ID NO: 52; a light chain variable region CDR2 of SEQ ID NO: 66; and a light chain variable region CDR3 of SEQ ID NO: 80,
(k) a heavy chain variable region CDR1 of SEQ ID NO: 11; a heavy chain variable region CDR2 of SEQ ID NO: 25; a heavy chain variable region CDR3 of SEQ ID NO: 39; a light chain variable region CDR1 of SEQ ID NO: 53; a light chain variable region CDR2 of SEQ ID NO: 67; and a light chain variable region CDR3 of SEQ ID NO: 81,
(l) a heavy chain variable region CDR1 of SEQ ID NO: 12; a heavy chain variable region CDR2 of SEQ ID NO: 26; a heavy chain variable region CDR3 of SEQ ID NO: 40; a light chain variable region CDR1 of SEQ ID NO: 54; a light chain variable region CDR2 of SEQ ID NO: 68; and a light chain variable region CDR3 of SEQ ID NO: 82,
(m) a heavy chain variable region CDR1 of SEQ ID NO: 13; a heavy chain variable region CDR2 of SEQ ID NO: 27; a heavy chain variable region CDR3 of SEQ ID NO: 41; a light chain variable region CDR1 of SEQ ID NO: 55; a light chain variable region CDR2 of SEQ ID NO: 69; and a light chain variable region CDR3 of SEQ ID NO: 83, or
(n) a heavy chain variable region CDR1 of SEQ ID NO: 14; a heavy chain variable region CDR2 of SEQ ID NO: 28; a heavy chain variable region CDR3 of SEQ ID NO: 42; a light chain variable region CDR1 of SEQ ID NO: 56; a light chain variable region CDR2 of SEQ ID NO: 70; and a light chain variable region CDR3 of SEQ ID NO: 84.

8. The method according to claim 1, wherein the ActRII receptor antagonist is an anti-ActRII receptor antibody or an antigen-binding portion thereof, and wherein the antibody comprises a full-length heavy chain amino acid sequence having at least 95% sequence identity to at least one sequence selected from the group consisting of SEQ ID NOs: 146-150 and 156-160 and a full-length light chain amino acid sequence having at least 95% sequence identity to at least one sequence selected from the group consisting of SEQ ID NOs: 141-145 and 151-155.

9. The method according to claim 1, wherein the ActRII receptor antagonist is an anti-ActRII receptor antibody or an antigen-binding portion thereof, and wherein the antibody or an antigen-binding portion thereof comprises:
(a) the variable heavy chain sequence of SEQ ID NO: 99 and variable light chain sequence of SEQ ID NO: 85;
(b) the variable heavy chain sequence of SEQ ID NO: 100 and variable light chain sequence of SEQ ID NO: 86;
(c) the variable heavy chain sequence of SEQ ID NO: 101 and variable light chain sequence of SEQ ID NO: 87;
(d) the variable heavy chain sequence of SEQ ID NO: 102 and variable light chain sequence of SEQ ID NO: 88;
(e) the variable heavy chain sequence of SEQ ID NO: 103 and variable light chain sequence of SEQ ID NO: 89;
(f) the variable heavy chain sequence of SEQ ID NO: 104 and variable light chain sequence of SEQ ID NO: 90;
(g) the variable heavy chain sequence of SEQ ID NO: 105 and variable light chain sequence of SEQ ID NO: 91;
(h) the variable heavy chain sequence of SEQ ID NO: 106 and variable light chain sequence of SEQ ID NO: 92;
(i) the variable heavy chain sequence of SEQ ID NO: 107 and variable light chain sequence of SEQ ID NO: 93;
(j) the variable heavy chain sequence of SEQ ID NO: 108 and variable light chain sequence of SEQ ID NO: 94;
(k) the variable heavy chain sequence of SEQ ID NO: 109 and variable light chain sequence of SEQ ID NO: 95;
(l) the variable heavy chain sequence of SEQ ID NO: 110 and variable light chain sequence of SEQ ID NO: 96;
(m) the variable heavy chain sequence of SEQ ID NO: 111 and variable light chain sequence of SEQ ID NO: 97; or
(n) the variable heavy chain sequence of SEQ ID NO: 112 and variable light chain sequence of SEQ ID NO: 98.

10. The method according to claim 9, wherein the anti-ActRII receptor antibody or an antigen-binding portion thereof is an anti-ActRII receptor antibody, and wherein the antibody comprises:
(a) the heavy chain sequence of SEQ ID NO: 146 and light chain sequence of SEQ ID NO: 141;
(b) the heavy chain sequence of SEQ ID NO: 147 and light chain sequence of SEQ ID NO: 142;
(c) the heavy chain sequence of SEQ ID NO: 148 and light chain sequence of SEQ ID NO: 143;
(d) the heavy chain sequence of SEQ ID NO: 149 and light chain sequence of SEQ ID NO: 144;
(e) the heavy chain sequence of SEQ ID NO: 150 and light chain sequence of SEQ ID NO: 145;
(f) the heavy chain sequence of SEQ ID NO: 156 and light chain sequence of SEQ ID NO: 151;
(g) the heavy chain sequence of SEQ ID NO: 157 and light chain sequence of SEQ ID NO: 152;
(h) the heavy chain sequence of SEQ ID NO: 158 and light chain sequence of SEQ ID NO: 153;
(i) the heavy chain sequence of SEQ ID NO: 159 and light chain sequence of SEQ ID NO: 154; or
(j) the heavy chain sequence of SEQ ID NO: 160 and light chain sequence of SEQ ID NO: 155.

11. The method according to claim 1, wherein the ActRII receptor antagonist is an anti-ActRII receptor antibody or an antigen-binding portion thereof, and wherein the antibody is encoded by pBW522 (DSM22873) or pBW524 (DSM22874).

12. A method for treating and/or preventing urinary incontinence, said method comprising administering an effective amount of bimagrumab to a subject having urinary incontinence or at risk for developing urinary incontinence.


SEQUENCE LISTING (194 sequences)

SEQ ID NO: 1 - protein, 10 aa, Artificial
  CDR
GYTFTSSYIN

SEQ ID NO: 2 - protein, 10 aa, Artificial
  CDR
GYTFTSSYIN

SEQ ID NO: 3 - protein, 10 aa, Artificial
  CDR
GYTFTSSYIN

SEQ ID NO: 4 - protein, 10 aa, Artificial
  CDR
GYTFTSSYIN

SEQ ID NO: 5 - protein, 10 aa, Artificial
  CDR
GYTFTSSYIN

SEQ ID NO: 6 - protein, 10 aa, Artificial
  CDR
GYTFTSSYIN

SEQ ID NO: 7 - protein, 10 aa, Artificial
  CDR
GYTFTSSYIN

SEQ ID NO: 8 - protein, 10 aa, Artificial
  CDR
GYTFTSSYIN

SEQ ID NO: 9 - protein, 10 aa, Artificial
  CDR
GYTFTSSYIN

SEQ ID NO: 10 - protein, 10 aa, Artificial
  CDR
GYTFTSSYIN

SEQ ID NO: 11 - protein, 10 aa, Artificial
  CDR
GYTFTSSYIN

SEQ ID NO: 12 - protein, 10 aa, Artificial
  CDR
GYTFTSSYIN

SEQ ID NO: 13 - protein, 10 aa, Artificial
  CDR
GYTFTSSYIN

SEQ ID NO: 14 - protein, 10 aa, Artificial
  CDR
GYTFTSSYIN

SEQ ID NO: 15 - protein, 17 aa, Artificial
  CDR
TINPVSGNTSYAQKFQG

SEQ ID NO: 16 - protein, 17 aa, Artificial
  CDR
TINPVSGNTSYAQKFQG

SEQ ID NO: 17 - protein, 17 aa, Artificial
  CDR
TINPVSGNTSYAQKFQG

SEQ ID NO: 18 - protein, 17 aa, Artificial
  CDR
TINPVSGNTSYAQKFQG

SEQ ID NO: 19 - protein, 17 aa, Artificial
  CDR
MINAPIGTTRYAQKFQG

SEQ ID NO: 20 - protein, 17 aa, Artificial
  CDR
QINAASGMTRYAQKFQG

SEQ ID NO: 21 - protein, 17 aa, Artificial
  CDR
MINAPIGTTRYAQKFQG

SEQ ID NO: 22 - protein, 17 aa, Artificial
  CDR
TINPVSGNTRYAQKFQG

SEQ ID NO: 23 - protein, 17 aa, Artificial
  CDR
TINPVSGSTSYAQKFQG

SEQ ID NO: 24 - protein, 17 aa, Artificial
  CDR
QINAASGMTRYAQKFQG

SEQ ID NO: 25 - protein, 17 aa, Artificial
  CDR
NINAAAGITLYAQKFQG

SEQ ID NO: 26 - protein, 17 aa, Artificial
  CDR
TINPPTGGTYYAQKFQG

SEQ ID NO: 27 - protein, 17 aa, Artificial
  CDR
GINPPAGTTSYAQKFQG

SEQ ID NO: 28 - protein, 17 aa, Artificial
  CDR
NINPATGHADYAQKFQG

SEQ ID NO: 29 - protein, 6 aa, Artificial
  CDR
GGWFDY

SEQ ID NO: 30 - protein, 6 aa, Artificial
  CDR
GGWFDY

SEQ ID NO: 31 - protein, 6 aa, Artificial
  CDR
GGWFDY

SEQ ID NO: 32 - protein, 6 aa, Artificial
  CDR
GGWFDY

SEQ ID NO: 33 - protein, 6 aa, Artificial
  CDR
GGWFDY

SEQ ID NO: 34 - protein, 6 aa, Artificial
  CDR
GGWFDY

SEQ ID NO: 35 - protein, 6 aa, Artificial
  CDR
GGWFDY

SEQ ID NO: 36 - protein, 6 aa, Artificial
  CDR
GGWFDY

SEQ ID NO: 37 - protein, 6 aa, Artificial
  CDR
GGWFDY

SEQ ID NO: 38 - protein, 6 aa, Artificial
  CDR
GGWFDY

SEQ ID NO: 39 - protein, 6 aa, Artificial
  CDR
GGWFDY

SEQ ID NO: 40 - protein, 6 aa, Artificial
  CDR
GGWFDY

SEQ ID NO: 41 - protein, 6 aa, Artificial
  CDR
GGWFDY

SEQ ID NO: 42 - protein, 6 aa, Artificial
  CDR
GGWFDY

SEQ ID NO: 43 - protein, 14 aa, Artificial
  CDR
TGTSSDVGSYNYVN

SEQ ID NO: 44 - protein, 14 aa, Artificial
  CDR
TGTSSDVGSYNYVN

SEQ ID NO: 45 - protein, 14 aa, Artificial
  CDR
TGTSSDVGSYNYVN

SEQ ID NO: 46 - protein, 14 aa, Artificial
  CDR
TGTSSDVGSYNYVN

SEQ ID NO: 47 - protein, 14 aa, Artificial
  CDR
TGTSSDVGSYNYVN

SEQ ID NO: 48 - protein, 14 aa, Artificial
  CDR
TGTSSDVGSYNYVN

SEQ ID NO: 49 - protein, 14 aa, Artificial
  CDR
TGTSSDVGSYNYVN

SEQ ID NO: 50 - protein, 14 aa, Artificial
  CDR
TGTSSDVGSYNYVN

SEQ ID NO: 51 - protein, 14 aa, Artificial
  CDR
TGTSSDVGSYNYVN

SEQ ID NO: 52 - protein, 14 aa, Artificial
  CDR
TGTSSDVGSYNYVN

SEQ ID NO: 53 - protein, 14 aa, Artificial
  CDR
TGTSSDVGSYNYVN

SEQ ID NO: 54 - protein, 14 aa, Artificial
  CDR
TGTSSDVGSYNYVN

SEQ ID NO: 55 - protein, 14 aa, Artificial
  CDR
TGTSSDVGSYNYVN

SEQ ID NO: 56 - protein, 14 aa, Artificial
  CDR
TGTSSDVGSYNYVN

SEQ ID NO: 57 - protein, 11 aa, Artificial
  CDR
LMIYGVSKRPS

SEQ ID NO: 58 - protein, 11 aa, Artificial
  CDR
LMIYGVSKRPS

SEQ ID NO: 59 - protein, 11 aa, Artificial
  CDR
LMIYGVSKRPS

SEQ ID NO: 60 - protein, 11 aa, Artificial
  CDR
LMIYGVSKRPS

SEQ ID NO: 61 - protein, 11 aa, Artificial
  CDR
LMIYGVSKRPS

SEQ ID NO: 62 - protein, 11 aa, Artificial
  CDR
LMIYGVSKRPS

SEQ ID NO: 63 - protein, 11 aa, Artificial
  CDR
LMIYGVSKRPS

SEQ ID NO: 64 - protein, 11 aa, Artificial
  CDR
LMIYGVSKRPS

SEQ ID NO: 65 - protein, 11 aa, Artificial
  CDR
LMIYGVSKRPS

SEQ ID NO: 66 - protein, 11 aa, Artificial
  CDR
LMIYGVSKRPS

SEQ ID NO: 67 - protein, 11 aa, Artificial
  CDR
LMIYGVSKRPS

SEQ ID NO: 68 - protein, 11 aa, Artificial
  CDR
LMIYGVSKRPS

SEQ ID NO: 69 - protein, 11 aa, Artificial
  CDR
LMIYGVSKRPS

SEQ ID NO: 70 - protein, 11 aa, Artificial
  CDR
LMIYGVSKRPS

SEQ ID NO: 71 - protein, 9 aa, Artificial
  CDR
QAWTSKMAG

SEQ ID NO: 72 - protein, 9 aa, Artificial
  CDR
SSYTRMGHP

SEQ ID NO: 73 - protein, 10 aa, Artificial
  CDR
ATYGKGVTPP

SEQ ID NO: 74 - protein, 10 aa, Artificial
  CDR
GTFAGGSYYG

SEQ ID NO: 75 - protein, 9 aa, Artificial
  CDR
QAWTSKMAG

SEQ ID NO: 76 - protein, 9 aa, Artificial
  CDR
QAWTSKMAG

SEQ ID NO: 77 - protein, 10 aa, Artificial
  CDR
GTFAGGSYYG

SEQ ID NO: 78 - protein, 10 aa, Artificial
  CDR
GTFAGGSYYG

SEQ ID NO: 79 - protein, 10 aa, Artificial
  CDR
GTFAGGSYYG

SEQ ID NO: 80 - protein, 10 aa, Artificial
  CDR
GTFAGGSYYG

SEQ ID NO: 81 - protein, 10 aa, Artificial
  CDR
GTFAGGSYYG

SEQ ID NO: 82 - protein, 10 aa, Artificial
  CDR
GTFAGGSYYG

SEQ ID NO: 83 - protein, 10 aa, Artificial
  CDR
GTFAGGSYYG

SEQ ID NO: 84 - protein, 10 aa, Artificial
  CDR
GTFAGGSYYG

SEQ ID NO: 85 - protein, 112 aa, Artificial
  VL
DIALTQPASVSGSPGQSITISCTGTSSDVGSYNYVNWYQQHPGKAPKLMIYGVSKRPSGVSNRFSGSKSGNTASLTISGLQAEDEADYYCQAWTSKMAGVFGGGTKLTVLGQ

SEQ ID NO: 86 - protein, 112 aa, Artificial
  VL
DIALTQPASVSGSPGQSITISCTGTSSDVGSYNYVNWYQQHPGKAPKLMIYGVSKRPSGVSNRFSGSKSGNTASLTISGLQAEDEADYYCSSYTRMGHPVFGGGTKLTVLGQ

SEQ ID NO: 87 - protein, 113 aa, Artificial
  VL
DIALTQPASVSGSPGQSITISCTGTSSDVGSYNYVNWYQQHPGKAPKLMIYGVSKRPSGVSNRFSGSKSGNTASLTISGLQAEDEADYYCATYGKGVTPPVFGGGTKLTVLGQ

SEQ ID NO: 88 - protein, 113 aa, Artificial
  VL
DIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 89 - protein, 112 aa, Artificial; identical to SEQ ID NO: 85
  VL

SEQ ID NO: 90 - protein, 112 aa, Artificial; identical to SEQ ID NO: 85
  VL

SEQ ID NO: 91 - protein, 113 aa, Artificial; identical to SEQ ID NO: 88
  VL

SEQ ID NO: 92 - protein, 113 aa, Artificial; identical to SEQ ID NO: 88
  VL

SEQ ID NO: 93 - protein, 113 aa, Artificial; identical to SEQ ID NO: 88
  VL

SEQ ID NO: 94 - protein, 113 aa, Artificial; identical to SEQ ID NO: 88
  VL

SEQ ID NO: 95 - protein, 113 aa, Artificial; identical to SEQ ID NO: 88
  VL

SEQ ID NO: 96 - protein, 113 aa, Artificial; identical to SEQ ID NO: 88
  VL

SEQ ID NO: 97 - protein, 113 aa, Artificial; identical to SEQ ID NO: 88
  VL

SEQ ID NO: 98 - protein, 113 aa, Artificial; identical to SEQ ID NO: 88
  VL

SEQ ID NO: 99 - protein, 115 aa, Artificial
  VH
QVQLVQSGAEVKKPGASVKVSCKASGYTFTSSYINWVRQAPGQGLEWMGTINPVSGNTSYAQKFQGRVTMTRDTSISTAYMELSSLRSEDTAVYYCARGGWFDYWGQGTLVTVSS

SEQ ID NO: 100 - protein, 115 aa, Artificial; identical to SEQ ID NO: 99
  VH

SEQ ID NO: 101 - protein, 115 aa, Artificial; identical to SEQ ID NO: 99
  VH

SEQ ID NO: 102 - protein, 115 aa, Artificial; identical to SEQ ID NO: 99
  VH

SEQ ID NO: 103 - protein, 115 aa, Artificial
  VH
QVQLVQSGAEVKKPGASVKVSCKASGYTFTSSYINWVRQAPGQGLEWMGMINAPIGTTRYAQKFQGRVTMTRDTSISTAYMELSSLRSEDTAVYYCARGGWFDYWGQGTLVTVSS

SEQ ID NO: 104 - protein, 115 aa, Artificial
  VH
QVQLVQSGAEVKKPGASVKVSCKASGYTFTSSYINWVRQAPGQGLEWMGQINAASGMTRYAQKFQGRVTMTRDTSISTAYMELSSLRSEDTAVYYCARGGWFDYWGQGTLVTVSS

SEQ ID NO: 105 - protein, 115 aa, Artificial; identical to SEQ ID NO: 103
  VH

SEQ ID NO: 106 - protein, 115 aa, Artificial
  VH
QVQLVQSGAEVKKPGASVKVSCKASGYTFTSSYINWVRQAPGQGLEWMGTINPVSGNTRYAQKFQGRVTMTRDTSISTAYMELSSLRSEDTAVYYCARGGWFDYWGQGTLVTVSS

SEQ ID NO: 107 - protein, 115 aa, Artificial
  VH
QVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSS

SEQ ID NO: 108 - protein, 115 aa, Artificial; identical to SEQ ID NO: 104
  VH

SEQ ID NO: 109 - protein, 115 aa, Artificial
  VH
QVQLVQSGAEVKKPGASVKVSCKASGYTFTSSYINWVRQAPGQGLEWMGNINAAAGITLYAQKFQGRVTMTRDTSISTAYMELSSLRSEDTAVYYCARGGWFDYWGQGTLVTVSS

SEQ ID NO: 110 - protein, 115 aa, Artificial
  VH
QVQLVQSGAEVKKPGASVKVSCKASGYTFTSSYINWVRQAPGQGLEWMGTINPPTGGTYYAQKFQGRVTMTRDTSISTAYMELSSLRSEDTAVYYCARGGWFDYWGQGTLVTVSS

SEQ ID NO: 111 - protein, 115 aa, Artificial
  VH
QVQLVQSGAEVKKPGASVKVSCKASGYTFTSSYINWVRQAPGQGLEWMGGINPPAGTTSYAQKFQGRVTMTRDTSISTAYMELSSLRSEDTAVYYCARGGWFDYWGQGTLVTVSS

SEQ ID NO: 112 - protein, 115 aa, Artificial
  VH
QVQLVQSGAEVKKPGASVKVSCKASGYTFTSSYINWVRQAPGQGLEWMGNINPATGHADYAQKFQGRVTMTRDTSISTAYMELSSLRSEDTAVYYCARGGWFDYWGQGTLVTVSS

SEQ ID NO: 113 - DNA, 336 bp, Artificial
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgccaggcttggacttctaagatggctggtgtgtttggcggcggcacgaagttaaccgttcttggccag

SEQ ID NO: 114 - DNA, 336 bp, Artificial
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgctcttcttatactcgtatgggtcatcctgtgtttggcggcggcacgaagttaaccgttcttggccag

SEQ ID NO: 115 - DNA, 339 bp, Artificial
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcgctacttatggtaagggtgttactcctcctgtgtttggcggcggcacgaagttaaccgttcttggccag

SEQ ID NO: 116 - DNA, 339 bp, Artificial
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgttcttggccag

SEQ ID NO: 117 - DNA, 336 bp, Artificial; identical to SEQ ID NO: 113
  VL

SEQ ID NO: 118 - DNA, 336 bp, Artificial; identical to SEQ ID NO: 113
  VL

SEQ ID NO: 119 - DNA, 339 bp, Artificial; identical to SEQ ID NO: 116
  VL

SEQ ID NO: 120 - DNA, 339 bp, Artificial; identical to SEQ ID NO: 116
  VL

SEQ ID NO: 121 - DNA, 339 bp, Artificial; identical to SEQ ID NO: 116
  VL

SEQ ID NO: 122 - DNA, 339 bp, Artificial; identical to SEQ ID NO: 116
  VL

SEQ ID NO: 123 - DNA, 339 bp, Artificial; identical to SEQ ID NO: 116
  VL

SEQ ID NO: 124 - DNA, 339 bp, Artificial
  VL
gatatcgcactgacccagccagcttcagtgagcggctcaccaggtcagagcattaccatctcgtgtactggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgttcttggccag

SEQ ID NO: 125 - DNA, 339 bp, Artificial; identical to SEQ ID NO: 116
  VL

SEQ ID NO: 126 - DNA, 339 bp, Artificial; identical to SEQ ID NO: 116
  VL

SEQ ID NO: 127 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggcaatacgtcttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 128 - DNA, 345 bp, Artificial; identical to SEQ ID NO: 127
  VH

SEQ ID NO: 129 - DNA, 345 bp, Artificial; identical to SEQ ID NO: 127
  VH

SEQ ID NO: 130 - DNA, 345 bp, Artificial; identical to SEQ ID NO: 127
  VH

SEQ ID NO: 131 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcatgattaatgctcctattggtactactcgttatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 132 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggccagattaatgctgcttctggtatgactcgttatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 133 - DNA, 345 bp, Artificial; identical to SEQ ID NO: 131
  VH

SEQ ID NO: 134 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggcaatacgcgttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 135 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggctctacgtcttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 136 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggccagattaatgctgcttctggtatgactcgttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 137 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 138 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactattaatcctcctactggaggtacttattatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 139 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 140 - DNA, 345 bp, Artificial
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 141 - protein, 217 aa, Artificial
  light chain
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 142 - protein, 217 aa, Artificial; identical to SEQ ID NO: 141
  light chain

SEQ ID NO: 143 - protein, 217 aa, Artificial; identical to SEQ ID NO: 141
  light chain

SEQ ID NO: 144 - protein, 217 aa, Artificial; identical to SEQ ID NO: 141
  light chain

SEQ ID NO: 145 - protein, 217 aa, Artificial; identical to SEQ ID NO: 141
  light chain

SEQ ID NO: 146 - protein, 445 aa, Artificial
  heavy chain
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 445 aa, Artificial
  heavy chain
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 445 aa, Artificial
  heavy chain
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 445 aa, Artificial
  heavy chain
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 445 aa, Artificial
  heavy chain
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 217 aa, Artificial; identical to SEQ ID NO: 141
  light chain

SEQ ID NO: 152 - protein, 217 aa, Artificial; identical to SEQ ID NO: 141
  light chain

SEQ ID NO: 153 - protein, 217 aa, Artificial; identical to SEQ ID NO: 141
  light chain

SEQ ID NO: 154 - protein, 217 aa, Artificial; identical to SEQ ID NO: 141
  light chain

SEQ ID NO: 155 - protein, 217 aa, Artificial; identical to SEQ ID NO: 141
  light chain

SEQ ID NO: 156 - protein, 441 aa, Artificial
  heavy chain
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 157 - protein, 441 aa, Artificial
  heavy chain
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 158 - protein, 441 aa, Artificial
  heavy chain
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 159 - protein, 441 aa, Artificial
  heavy chain
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 160 - protein, 441 aa, Artificial
  heavy chain
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 161 - DNA, 651 bp, Artificial
  light chain
cagagcgccctgacccagcccgccagcgtgtccggcagcccaggccagtctatcacaatcagctgcaccggcacctccagcgacgtgggcagctacaactacgtgaactggtatcagcagcaccccggcaaggcccccaagctgatgatctacggcgtgagcaagaggcccagcggcgtgtccaacaggttcagcggcagcaagagcggcaacaccgccagcctgacaatcagtgggctgcaggctgaggacgaggccgactactactgcggcacctttgccggcggatcatactacggcgtgttcggcggagggaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 162 - DNA, 651 bp, Artificial; identical to SEQ ID NO: 161
  light chain

SEQ ID NO: 163 - DNA, 651 bp, Artificial
  light chain
cagagcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 164 - DNA, 651 bp, Artificial; identical to SEQ ID NO: 163
  light chain

SEQ ID NO: 165 - DNA, 651 bp, Artificial; identical to SEQ ID NO: 163
  light chain

SEQ ID NO: 166 - DNA, 1335 bp, Artificial
  heavy chain
caggtgcagctggtgcagagcggagctgaggtgaagaagccaggcgccagcgtcaaggtgtcctgcaaggccagcggctacaccttcaccagcagctacatcaactgggtccgccaggctcctgggcagggactggagtggatgggcaccatcaaccccgtgtccggcagcaccagctacgcccagaagttccagggcagagtcaccatgaccagggacaccagcatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggcccccagcagcaagagcacctccggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagcccccgaagctgcaggcggcccttccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaggtctccaacaaggccctgcctgcccccatcgaaaagaccatcagcaaggccaagggccagccacgggagccccaggtgtacaccctgcccccttctcgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcacccggcaag

SEQ ID NO: 167 - DNA, 1335 bp, Artificial
  heavy chain
caggtgcagctggtgcagagcggagctgaggtgaagaagccaggcgccagcgtcaaggtgtcctgcaaggccagcggctacaccttcaccagcagctacatcaactgggtgcgccaggctccagggcagggactggagtggatgggccagatcaacgccgccagcggcatgaccagatacgcccagaagttccagggcagagtcacaatgaccagggacacctctatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggcccccagcagcaagagcacctccggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagcccccgaagctgcaggcggcccttccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaggtctccaacaaggccctgcctgcccccatcgaaaagaccatcagcaaggccaagggccagccacgggagccccaggtgtacaccctgcccccttctcgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcacccggcaag

SEQ ID NO: 168 - DNA, 1335 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 169 - DNA, 1335 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 170 - DNA, 1335 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 171 - DNA, 651 bp, Artificial; identical to SEQ ID NO: 161
  light chain

SEQ ID NO: 172 - DNA, 651 bp, Artificial; identical to SEQ ID NO: 161
  light chain

SEQ ID NO: 173 - DNA, 651 bp, Artificial; identical to SEQ ID NO: 163
  light chain

SEQ ID NO: 174 - DNA, 651 bp, Artificial; identical to SEQ ID NO: 163
  light chain

SEQ ID NO: 175 - DNA, 651 bp, Artificial; identical to SEQ ID NO: 163
  light chain

SEQ ID NO: 176 - DNA, 1323 bp, Artificial
  heavy chain
caggtgcagctggtgcagagcggagctgaggtgaagaagccaggcgccagcgtcaaggtgtcctgcaaggccagcggctacaccttcaccagcagctacatcaactgggtccgccaggctcctgggcagggactggagtggatgggcaccatcaaccccgtgtccggcagcaccagctacgcccagaagttccagggcagagtcaccatgaccagggacaccagcatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcaacttcggcacccagacctacacctgcaacgtggaccacaagcccagcaacaccaaggtggacaagaccgtggagaggaagtgctgcgtggagtgccccccctgcccagcccccccagtggccggaccctccgtgttcctgttcccccccaagcccaaggacaccctgatgatcagcaggacccccgaggtgacctgcgtggtggtggacgtgagccacgaggacccagaggtgcagttcaactggtacgtggacggcgtggaggtgcacaacgccaagaccaagcccagagaggaacagtttaacagcaccttcagggtggtgtccgtgctgaccgtggtgcaccaggactggctgaacggcaaagagtacaagtgcaaggtctccaacaagggcctgccagcccccatcgagaaaaccatcagcaagaccaagggccagccacgggagccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaag

SEQ ID NO: 177 - DNA, 1323 bp, Artificial
  heavy chain
caggtgcagctggtgcagagcggagctgaggtgaagaagccaggcgccagcgtcaaggtgtcctgcaaggccagcggctacaccttcaccagcagctacatcaactgggtgcgccaggctccagggcagggactggagtggatgggccagatcaacgccgccagcggcatgaccagatacgcccagaagttccagggcagagtcacaatgaccagggacacctctatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcaacttcggcacccagacctacacctgcaacgtggaccacaagcccagcaacaccaaggtggacaagaccgtggagaggaagtgctgcgtggagtgccccccctgcccagcccccccagtggccggaccctccgtgttcctgttcccccccaagcccaaggacaccctgatgatcagcaggacccccgaggtgacctgcgtggtggtggacgtgagccacgaggacccagaggtgcagttcaactggtacgtggacggcgtggaggtgcacaacgccaagaccaagcccagagaggaacagtttaacagcaccttcagggtggtgtccgtgctgaccgtggtgcaccaggactggctgaacggcaaagagtacaagtgcaaggtctccaacaagggcctgccagcccccatcgagaaaaccatcagcaagaccaagggccagccacgggagccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaag

SEQ ID NO: 178 - DNA, 1323 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 179 - DNA, 1323 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 180 - DNA, 1323 bp, Artificial
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 181 - protein, 512 aa, Homo sapiens [sequence omitted]

SEQ ID NO: 182 - protein, 116 aa, Homo sapiens
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 183 - protein, 15 aa, Homo sapiens
IELVKKGSWLDDFNS

SEQ ID NO: 184 - protein, 15 aa, Homo sapiens
VKKGSWLDDFNSYDR

SEQ ID NO: 185 - protein, 15 aa, Homo sapiens
GSWLDDFNSYDRQES

SEQ ID NO: 186 - protein, 9 aa, Homo sapiens
GCWLDDFNC

SEQ ID NO: 187 - protein, 15 aa, Homo sapiens
CEGEQDKRLHCYASW

SEQ ID NO: 188 - protein, 6 aa, Homo sapiens
WLDDFN

SEQ ID NO: 189 - protein, 5 aa, Homo sapiens
EQDKR

SEQ ID NO: 190 - protein, 11 aa, Homo sapiens
KGCWLDDFNCY

SEQ ID NO: 191 - protein, 13 aa, Human
CIYYNANWELERT

SEQ ID NO: 192 - protein, 11 aa, Human
YFCCCEGNFCN

SEQ ID NO: 193 - protein, 216 aa, Artificial Sequence
  Light Chain
DIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 194 - protein, 445 aa, Artificial
  Heavy Chain
QVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK